SEQUENCE LISTING (372 sequences)

SEQ ID NO: 1 - protein, 2527 aa, Homo sapiens
  ref <308>: DATABASE ACCESSION NUMBER: GenBank / NP_940980.3
  ref <309>: DATABASE ENTRY DATE: 2017-10-03
  ref <313>: RELEVANT RESIDUES IN SEQ ID NO: (1)..(2527)
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 2 - DNA, 30 bp, Artificial Sequence
  Synthetic polynucleotide
acgttggatgccattttatgacgcagcgag

SEQ ID NO: 3 - DNA, 30 bp, Artificial Sequence
  Synthetic polynucleotide
acgttggatgggcttcatggcatcaacttc

SEQ ID NO: 4 - DNA, 17 bp, Artificial Sequence
  Synthetic polynucleotide
tcaacttcagcctgtcc

SEQ ID NO: 5 - DNA, 29 bp, Artificial Sequence
  Synthetic polynucleotide
acgttggatgaaacagtgtttcaagaatg

SEQ ID NO: 6 - DNA, 30 bp, Artificial Sequence
  Synthetic polynucleotide
acgttggatgaaaaagcgcccctagttctc

SEQ ID NO: 7 - DNA, 17 bp, Artificial Sequence
  Synthetic polynucleotide
agctgctaggaccagtt

SEQ ID NO: 8 - DNA, 30 bp, Artificial Sequence
  Synthetic polynucleotide
acgttggatgattctacctggcccaatatg

SEQ ID NO: 9 - DNA, 30 bp, Artificial Sequence
  Synthetic polynucleotide
acgttggatgatacattctcctgaagtggc

SEQ ID NO: 10 - DNA, 17 bp, Artificial Sequence
  Synthetic polynucleotide
cctgaagtggctgaaag

SEQ ID NO: 11 - DNA, 30 bp, Artificial Sequence
  Synthetic polynucleotide
acgttggatggggttttgtgtctttccctc

SEQ ID NO: 12 - DNA, 30 bp, Artificial Sequence
  Synthetic polynucleotide
acgttggatgcatccaaatgtgtgccaacg

SEQ ID NO: 13 - DNA, 18 bp, Artificial Sequence
  Synthetic polynucleotide
acaggggaagaagaagcg

SEQ ID NO: 14 - DNA, 30 bp, Artificial Sequence
  Synthetic polynucleotide
acgttggatgtcccgtaattgaccggaaac

SEQ ID NO: 15 - DNA, 30 bp, Artificial Sequence
  Synthetic polynucleotide
acgttggatgaagtgaactgcgtgaggaag

SEQ ID NO: 16 - DNA, 19 bp, Artificial Sequence
  Synthetic polynucleotide
agctgattttctctcacta

SEQ ID NO: 17 - DNA, 30 bp, Artificial Sequence
  Synthetic polynucleotide
acgttggatgctgtccatagaagagtcagg

SEQ ID NO: 18 - DNA, 30 bp, Artificial Sequence
  Synthetic polynucleotide
acgttggatgctcaggcagcgatggaaatt

SEQ ID NO: 19 - DNA, 19 bp, Artificial Sequence
  Synthetic polynucleotide
acctgaagatgtgctgtct

SEQ ID NO: 20 - DNA, 30 bp, Artificial Sequence
  Synthetic polynucleotide
acgttggatgacttcaatcccatatgaggc

SEQ ID NO: 21 - DNA, 30 bp, Artificial Sequence
  Synthetic polynucleotide
acgttggatgtgctgaaaggtctagtgatg

SEQ ID NO: 22 - DNA, 19 bp, Artificial Sequence
  Synthetic polynucleotide
ggaagccagagaagaaatg

SEQ ID NO: 23 - DNA, 30 bp, Artificial Sequence
  Synthetic polynucleotide
acgttggatggcaaaatagccctgttgtgg

SEQ ID NO: 24 - DNA, 30 bp, Artificial Sequence
  Synthetic polynucleotide
acgttggatgcttaaaaagtgcacgcagtc

SEQ ID NO: 25 - DNA, 20 bp, Artificial Sequence
  Synthetic polynucleotide
aagtgcacgcagtctattag

SEQ ID NO: 26 - DNA, 30 bp, Artificial Sequence
  Synthetic polynucleotide
acgttggatggagaaaacagaacttaccag

SEQ ID NO: 27 - DNA, 30 bp, Artificial Sequence
  Synthetic polynucleotide
acgttggatgggaggtagaatagtagaggg

SEQ ID NO: 28 - DNA, 20 bp, Artificial Sequence
  Synthetic polynucleotide
ccgagggtttgaagtttcca

SEQ ID NO: 29 - DNA, 30 bp, Artificial Sequence
  Synthetic polynucleotide
acgttggatgttgttttggctggaagcctg

SEQ ID NO: 30 - DNA, 30 bp, Artificial Sequence
  Synthetic polynucleotide
acgttggatgtatgagagtcctacctgcac

SEQ ID NO: 31 - DNA, 21 bp, Artificial Sequence
  Synthetic polynucleotide
cccactacctgcacgtgcttg

SEQ ID NO: 32 - DNA, 30 bp, Artificial Sequence
  Synthetic polynucleotide
acgttggatgactttatggttctagggagg

SEQ ID NO: 33 - DNA, 30 bp, Artificial Sequence
  Synthetic polynucleotide
acgttggatgtgaaggcagagggttttcac

SEQ ID NO: 34 - DNA, 21 bp, Artificial Sequence
  Synthetic polynucleotide
tttcactctcccagaataaga

SEQ ID NO: 35 - DNA, 30 bp, Artificial Sequence
  Synthetic polynucleotide
acgttggatgcagagaatgcagcattgcag

SEQ ID NO: 36 - DNA, 30 bp, Artificial Sequence
  Synthetic polynucleotide
acgttggatgatgcctctgtaagaatcccc

SEQ ID NO: 37 - DNA, 22 bp, Artificial Sequence
  Synthetic polynucleotide
ggatgagggccaaacagctgag

SEQ ID NO: 38 - DNA, 30 bp, Artificial Sequence
  Synthetic polynucleotide
acgttggatgcccacaatgtgctgcttttc

SEQ ID NO: 39 - DNA, 30 bp, Artificial Sequence
  Synthetic polynucleotide
acgttggatgcattctacagcagtactgag

SEQ ID NO: 40 - DNA, 22 bp, Artificial Sequence
  Synthetic polynucleotide
ggagggatggcagcattgggat

SEQ ID NO: 41 - DNA, 30 bp, Artificial Sequence
  Synthetic polynucleotide
acgttggatgtattcagtacctaccaggtc

SEQ ID NO: 42 - DNA, 30 bp, Artificial Sequence
  Synthetic polynucleotide
acgttggatgcactcaagtccaagatgctg

SEQ ID NO: 43 - DNA, 23 bp, Artificial Sequence
  Synthetic polynucleotide
accttaagttcaaagatttccag

SEQ ID NO: 44 - DNA, 30 bp, Artificial Sequence
  Synthetic polynucleotide
acgttggatgcctctgttgcaagtgttttg

SEQ ID NO: 45 - DNA, 30 bp, Artificial Sequence
  Synthetic polynucleotide
acgttggatgctgctcccaatagaagcaag

SEQ ID NO: 46 - DNA, 23 bp, Artificial Sequence
  Synthetic polynucleotide
agatgctgttattctgatcacac

SEQ ID NO: 47 - DNA, 30 bp, Artificial Sequence
  Synthetic polynucleotide
acgttggatgatgagacagacctgatcacc

SEQ ID NO: 48 - DNA, 30 bp, Artificial Sequence
  Synthetic polynucleotide
acgttggatgcagtactgctgtagaatggg

SEQ ID NO: 49 - DNA, 23 bp, Artificial Sequence
  Synthetic polynucleotide
actgctgtagaatggggataaaa

SEQ ID NO: 50 - DNA, 30 bp, Artificial Sequence
  Synthetic polynucleotide
acgttggatggaactcacctaaatctcaag

SEQ ID NO: 51 - DNA, 30 bp, Artificial Sequence
  Synthetic polynucleotide
acgttggatgattcggttataaggcacagc

SEQ ID NO: 52 - DNA, 24 bp, Artificial Sequence
  Synthetic polynucleotide
gtagaaggcacagccttttttaat

SEQ ID NO: 53 - DNA, 30 bp, Artificial Sequence
  Synthetic polynucleotide
acgttggatgttcttttccccacccacttg

SEQ ID NO: 54 - DNA, 30 bp, Artificial Sequence
  Synthetic polynucleotide
acgttggatgaagacgatcaacagaggcac

SEQ ID NO: 55 - DNA, 24 bp, Artificial Sequence
  Synthetic polynucleotide
gtagttttgccttgaaataacttg

SEQ ID NO: 56 - DNA, 30 bp, Artificial Sequence
  Synthetic polynucleotide
acgttggatgttttgggccaagttgtggac

SEQ ID NO: 57 - DNA, 30 bp, Artificial Sequence
  Synthetic polynucleotide
acgttggatgcagagtttctccttcaccac

SEQ ID NO: 58 - DNA, 25 bp, Artificial Sequence
  Synthetic polynucleotide
cgtcaccacaaatatcaatctccag

SEQ ID NO: 59 - DNA, 30 bp, Artificial Sequence
  Synthetic polynucleotide
acgttggatgttgacagtgaaagtggaagg

SEQ ID NO: 60 - DNA, 30 bp, Artificial Sequence
  Synthetic polynucleotide
acgttggatgttttccacatctctacgcga

SEQ ID NO: 61 - DNA, 25 bp, Artificial Sequence
  Synthetic polynucleotide
cttacacatctctacgcgaaataat

SEQ ID NO: 62 - DNA, 30 bp, Artificial Sequence
  Synthetic polynucleotide
acgttggatgactgagtgaatcatctgaag

SEQ ID NO: 63 - DNA, 30 bp, Artificial Sequence
  Synthetic polynucleotide
acgttggatgttaataggggcccatttttg

SEQ ID NO: 64 - DNA, 25 bp, Artificial Sequence
  Synthetic polynucleotide
ggaggggcccatttttgatcatgaa

SEQ ID NO: 65 - DNA, 30 bp, Artificial Sequence
  Synthetic polynucleotide
acgttggatgaaaccactgagggtccaatg

SEQ ID NO: 66 - DNA, 30 bp, Artificial Sequence
  Synthetic polynucleotide
acgttggatgacacatttggacttgcacag

SEQ ID NO: 67 - DNA, 26 bp, Artificial Sequence
  Synthetic polynucleotide
ttggattgctaatcttgatgtctctc

SEQ ID NO: 68 - DNA, 30 bp, Artificial Sequence
  Synthetic polynucleotide
acgttggatgagagcatttggatttacctc

SEQ ID NO: 69 - DNA, 30 bp, Artificial Sequence
  Synthetic polynucleotide
acgttggatgcagaggacagctctcatttc

SEQ ID NO: 70 - DNA, 26 bp, Artificial Sequence
  Synthetic polynucleotide
cttgcttgacttaaatactgaaggat

SEQ ID NO: 71 - DNA, 30 bp, Artificial Sequence
  Synthetic polynucleotide
acgttggatgcttcctctgctttcttcatc

SEQ ID NO: 72 - DNA, 30 bp, Artificial Sequence
  Synthetic polynucleotide
acgttggatgactctgttgaagaaatgggc

SEQ ID NO: 73 - DNA, 26 bp, Artificial Sequence
  Synthetic polynucleotide
ggggcattatatagttttaatgatgg

SEQ ID NO: 74 - DNA, 30 bp, Artificial Sequence
  Synthetic polynucleotide
acgttggatgcttgattttggctgacctgc

SEQ ID NO: 75 - DNA, 30 bp, Artificial Sequence
  Synthetic polynucleotide
acgttggatgaggagaaactctggagcttg

SEQ ID NO: 76 - DNA, 27 bp, Artificial Sequence
  Synthetic polynucleotide
aaacttccaactcatcattattcaaca

SEQ ID NO: 77 - DNA, 30 bp, Artificial Sequence; identical to SEQ ID NO: 12
  Synthetic polynucleotide

SEQ ID NO: 78 - DNA, 30 bp, Artificial Sequence; identical to SEQ ID NO: 11
  Synthetic polynucleotide

SEQ ID NO: 79 - DNA, 17 bp, Artificial Sequence
  Synthetic polynucleotide
tttccctccaggctcgc

SEQ ID NO: 80 - DNA, 30 bp, Artificial Sequence
  Synthetic polynucleotide
acgttggatgatgtgatgctagagagagcg

SEQ ID NO: 81 - DNA, 30 bp, Artificial Sequence
  Synthetic polynucleotide
acgttggatgttggcatctgctcccaatag

SEQ ID NO: 82 - DNA, 17 bp, Artificial Sequence
  Synthetic polynucleotide
caagcattcaaccatga

SEQ ID NO: 83 - DNA, 30 bp, Artificial Sequence
  Synthetic polynucleotide
acgttggatgtgccatgcactcatttccag

SEQ ID NO: 84 - DNA, 30 bp, Artificial Sequence
  Synthetic polynucleotide
acgttggatgatacctctctcaaacagcac

SEQ ID NO: 85 - DNA, 17 bp, Artificial Sequence
  Synthetic polynucleotide
aacagcacatgtaaagc

SEQ ID NO: 86 - DNA, 30 bp, Artificial Sequence
  Synthetic polynucleotide
acgttggatgtcacgagatccactattcag

SEQ ID NO: 87 - DNA, 30 bp, Artificial Sequence
  Synthetic polynucleotide
acgttggatggccagtctcctaaaaggaag

SEQ ID NO: 88 - DNA, 18 bp, Artificial Sequence
  Synthetic polynucleotide
ctgagaaagagagcagtc

SEQ ID NO: 89 - DNA, 30 bp, Artificial Sequence
  Synthetic polynucleotide
acgttggatgtagcactgaatttgccaacc

SEQ ID NO: 90 - DNA, 30 bp, Artificial Sequence
  Synthetic polynucleotide
acgttggatgggaatcagctgtccaacaac

SEQ ID NO: 91 - DNA, 19 bp, Artificial Sequence
  Synthetic polynucleotide
tccaacaacaagctgatct

SEQ ID NO: 92 - DNA, 30 bp, Artificial Sequence
  Synthetic polynucleotide
acgttggatggccaaagcatcagattcctc

SEQ ID NO: 93 - DNA, 30 bp, Artificial Sequence
  Synthetic polynucleotide
acgttggatgaactcctgaataagcgaggg

SEQ ID NO: 94 - DNA, 19 bp, Artificial Sequence
  Synthetic polynucleotide
caagggttccctgccatac

SEQ ID NO: 95 - DNA, 30 bp, Artificial Sequence
  Synthetic polynucleotide
acgttggatgcgttggcacacatttggatg

SEQ ID NO: 96 - DNA, 30 bp, Artificial Sequence
  Synthetic polynucleotide
acgttggatgcctcgcttattcaggagttc

SEQ ID NO: 97 - DNA, 19 bp, Artificial Sequence
  Synthetic polynucleotide
aggagttccttggtgattt

SEQ ID NO: 98 - DNA, 30 bp, Artificial Sequence
  Synthetic polynucleotide
acgttggatgaaaggaccaagccaagaagg

SEQ ID NO: 99 - DNA, 29 bp, Artificial Sequence
  Synthetic polynucleotide
acgttggatgtggatgtggccaacaatag

SEQ ID NO: 100 - DNA, 19 bp, Artificial Sequence
  Synthetic polynucleotide
aagccttggaggattttgt

SEQ ID NO: 101 - DNA, 30 bp, Artificial Sequence
  Synthetic polynucleotide
acgttggatgggagaattttaccgagatgc

SEQ ID NO: 102 - DNA, 30 bp, Artificial Sequence
  Synthetic polynucleotide
acgttggatgcttaccaaggaattggaatg

SEQ ID NO: 103 - DNA, 20 bp, Artificial Sequence
  Synthetic polynucleotide
ggaaaatttggtgagcaacg

SEQ ID NO: 104 - DNA, 30 bp, Artificial Sequence
  Synthetic polynucleotide
acgttggatgatcttggaatgcaaagtgcc

SEQ ID NO: 105 - DNA, 30 bp, Artificial Sequence
  Synthetic polynucleotide
acgttggatgtcccacacatttaggacgag

SEQ ID NO: 106 - DNA, 21 bp, Artificial Sequence
  Synthetic polynucleotide
taggccagtctttcacatcta

SEQ ID NO: 107 - DNA, 30 bp, Artificial Sequence
  Synthetic polynucleotide
acgttggatgccctgaaagcatgtaaggtg

SEQ ID NO: 108 - DNA, 30 bp, Artificial Sequence
  Synthetic polynucleotide
acgttggatgtccccattgtgagaactctg

SEQ ID NO: 109 - DNA, 21 bp, Artificial Sequence
  Synthetic polynucleotide
cccgactatatgaaatgcctt

SEQ ID NO: 110 - DNA, 30 bp, Artificial Sequence
  Synthetic polynucleotide
acgttggatgtgagctctttcttacccttc

SEQ ID NO: 111 - DNA, 30 bp, Artificial Sequence
  Synthetic polynucleotide
acgttggatgtgtacctgagaacctcactg

SEQ ID NO: 112 - DNA, 21 bp, Artificial Sequence
  Synthetic polynucleotide
tgtggtagagaaactggagca

SEQ ID NO: 113 - DNA, 30 bp, Artificial Sequence
  Synthetic polynucleotide
acgttggatgctgtttttaactccagattg

SEQ ID NO: 114 - DNA, 30 bp, Artificial Sequence
  Synthetic polynucleotide
acgttggatgggaggagatctaaggtcttc

SEQ ID NO: 115 - DNA, 22 bp, Artificial Sequence
  Synthetic polynucleotide
acactggcattatgaactgtta

SEQ ID NO: 116 - DNA, 30 bp, Artificial Sequence
  Synthetic polynucleotide
acgttggatggtgagtgcattctggtgaag

SEQ ID NO: 117 - DNA, 30 bp, Artificial Sequence
  Synthetic polynucleotide
acgttggatggatgccctaagccagaaatg

SEQ ID NO: 118 - DNA, 22 bp, Artificial Sequence
  Synthetic polynucleotide
tgagccagaaatgctgtataag

SEQ ID NO: 119 - DNA, 30 bp, Artificial Sequence; identical to SEQ ID NO: 74
  Synthetic polynucleotide

SEQ ID NO: 120 - DNA, 30 bp, Artificial Sequence; identical to SEQ ID NO: 75
  Synthetic polynucleotide

SEQ ID NO: 121 - DNA, 23 bp, Artificial Sequence
  Synthetic polynucleotide
gttccaactcatcattattcaac

SEQ ID NO: 122 - DNA, 30 bp, Artificial Sequence
  Synthetic polynucleotide
acgttggatgcaggccagtttaatatatag

SEQ ID NO: 123 - DNA, 30 bp, Artificial Sequence
  Synthetic polynucleotide
acgttggatgtagcccaataccagcatgac

SEQ ID NO: 124 - DNA, 23 bp, Artificial Sequence
  Synthetic polynucleotide
ccaataccagcatgacattttta

SEQ ID NO: 125 - DNA, 30 bp, Artificial Sequence
  Synthetic polynucleotide
acgttggatgtatttcgcgtagagatgtgg

SEQ ID NO: 126 - DNA, 30 bp, Artificial Sequence
  Synthetic polynucleotide
acgttggatgctattggcaaagcaatctgg

SEQ ID NO: 127 - DNA, 23 bp, Artificial Sequence
  Synthetic polynucleotide
gtctaggagcttaaaatactgtg

SEQ ID NO: 128 - DNA, 30 bp, Artificial Sequence
  Synthetic polynucleotide
acgttggatgttctggcactgaaactctcc

SEQ ID NO: 129 - DNA, 30 bp, Artificial Sequence
  Synthetic polynucleotide
acgttggatgccacatttcatccctatcag

SEQ ID NO: 130 - DNA, 24 bp, Artificial Sequence
  Synthetic polynucleotide
gccctatcagagaactttcttgag

SEQ ID NO: 131 - DNA, 30 bp, Artificial Sequence
  Synthetic polynucleotide
acgttggatgaatgagagctgtcctctgtc

SEQ ID NO: 132 - DNA, 30 bp, Artificial Sequence
  Synthetic polynucleotide
acgttggatggcatggttgctacacatcac

SEQ ID NO: 133 - DNA, 24 bp, Artificial Sequence
  Synthetic polynucleotide
gcggaatgcaagcatttggctggg

SEQ ID NO: 134 - DNA, 31 bp, Artificial Sequence
  Synthetic polynucleotide
acgttggatggtatactttatggttctaggg

SEQ ID NO: 135 - DNA, 30 bp, Artificial Sequence; identical to SEQ ID NO: 33
  Synthetic polynucleotide

SEQ ID NO: 136 - DNA, 25 bp, Artificial Sequence
  Synthetic polynucleotide
gtttgattccttgttttcttttacc

SEQ ID NO: 137 - DNA, 30 bp, Artificial Sequence
  Synthetic polynucleotide
acgttggatgacattgccactcatgaggac

SEQ ID NO: 138 - DNA, 30 bp, Artificial Sequence
  Synthetic polynucleotide
acgttggatgtgaaaggagaacatggttac

SEQ ID NO: 139 - DNA, 25 bp, Artificial Sequence
  Synthetic polynucleotide
gtgatctctttaaattcaggcaata

SEQ ID NO: 140 - DNA, 30 bp, Artificial Sequence
  Synthetic polynucleotide
acgttggatgtcttcttccccctcatcatc

SEQ ID NO: 141 - DNA, 29 bp, Artificial Sequence
  Synthetic polynucleotide
acgttggatgtcaagctgagactattttc

SEQ ID NO: 142 - DNA, 25 bp, Artificial Sequence
  Synthetic polynucleotide
aaatcaagatttagaggaaaagaat

SEQ ID NO: 143 - DNA, 30 bp, Artificial Sequence
  Synthetic polynucleotide
acgttggatgaaagaatatggttgtgcccc

SEQ ID NO: 144 - DNA, 30 bp, Artificial Sequence
  Synthetic polynucleotide
acgttggatggcagaagtaggcctttcttg

SEQ ID NO: 145 - DNA, 26 bp, Artificial Sequence
  Synthetic polynucleotide
tctttcaaacactgtttaattaattt

SEQ ID NO: 146 - DNA, 30 bp, Artificial Sequence
  Synthetic polynucleotide
acgttggatggccttgtcgccaatacattc

SEQ ID NO: 147 - DNA, 30 bp, Artificial Sequence
  Synthetic polynucleotide
acgttggatgccctttaacttaggaagcag

SEQ ID NO: 148 - DNA, 26 bp, Artificial Sequence
  Synthetic polynucleotide
gtgagtaatggctccattttttagaa

SEQ ID NO: 149 - DNA, 30 bp, Artificial Sequence
  Synthetic polynucleotide
acgttggatgtgtgcatttaggcatgccag

SEQ ID NO: 150 - DNA, 30 bp, Artificial Sequence
  Synthetic polynucleotide
acgttggatgcaaagctgctaggaccagtt

SEQ ID NO: 151 - DNA, 27 bp, Artificial Sequence
  Synthetic polynucleotide
acctatgatgaaattctgtatcctccc

SEQ ID NO: 152 - DNA, 30 bp, Artificial Sequence
  Synthetic polynucleotide
acgttggatgggttttgtgtctttccctcc

SEQ ID NO: 153 - DNA, 30 bp, Artificial Sequence
  Synthetic polynucleotide
acgttggatggctttgcgttgcttctcatc

SEQ ID NO: 154 - DNA, 17 bp, Artificial Sequence
  Synthetic polynucleotide
atccaaatgtgtgccaa

SEQ ID NO: 155 - DNA, 30 bp, Artificial Sequence
  Synthetic polynucleotide
acgttggatgattctatagtactcatcccc

SEQ ID NO: 156 - DNA, 30 bp, Artificial Sequence
  Synthetic polynucleotide
acgttggatgctgaggtcatagacagcaag

SEQ ID NO: 157 - DNA, 17 bp, Artificial Sequence
  Synthetic polynucleotide
ggtacaatgctcgctgc

SEQ ID NO: 158 - DNA, 30 bp, Artificial Sequence; identical to SEQ ID NO: 39
  Synthetic polynucleotide

SEQ ID NO: 159 - DNA, 30 bp, Artificial Sequence; identical to SEQ ID NO: 38
  Synthetic polynucleotide

SEQ ID NO: 160 - DNA, 18 bp, Artificial Sequence
  Synthetic polynucleotide
ctcccaatgctgccatca

SEQ ID NO: 161 - DNA, 30 bp, Artificial Sequence
  Synthetic polynucleotide
acgttggatgcacatcagtgaggttctcag

SEQ ID NO: 162 - DNA, 30 bp, Artificial Sequence
  Synthetic polynucleotide
acgttggatgcagtgaaatgtccaactctg

SEQ ID NO: 163 - DNA, 19 bp, Artificial Sequence
  Synthetic polynucleotide
ttgtcatataaccagctgt

SEQ ID NO: 164 - DNA, 30 bp, Artificial Sequence
  Synthetic polynucleotide
acgttggatgccgcacactggaaatctttg

SEQ ID NO: 165 - DNA, 30 bp, Artificial Sequence; identical to SEQ ID NO: 42
  Synthetic polynucleotide

SEQ ID NO: 166 - DNA, 19 bp, Artificial Sequence
  Synthetic polynucleotide
gatgctgatctgattatgg

SEQ ID NO: 167 - DNA, 30 bp, Artificial Sequence
  Synthetic polynucleotide
acgttggatgccggtcaattacgggaaatt

SEQ ID NO: 168 - DNA, 30 bp, Artificial Sequence
  Synthetic polynucleotide
acgttggatggctgattccagactgctatg

SEQ ID NO: 169 - DNA, 19 bp, Artificial Sequence
  Synthetic polynucleotide
ggcggagcgtaaaaatgtg

SEQ ID NO: 170 - DNA, 30 bp, Artificial Sequence
  Synthetic polynucleotide
acgttggatgggaaattgtgcttcatgtgc

SEQ ID NO: 171 - DNA, 30 bp, Artificial Sequence
  Synthetic polynucleotide
acgttggatgtgctacttacaaggaatcgc

SEQ ID NO: 172 - DNA, 20 bp, Artificial Sequence
  Synthetic polynucleotide
ggatcgctagggaatgtaaa

SEQ ID NO: 173 - DNA, 30 bp, Artificial Sequence
  Synthetic polynucleotide
acgttggatggtcagattttcaagacagcc

SEQ ID NO: 174 - DNA, 30 bp, Artificial Sequence
  Synthetic polynucleotide
acgttggatgttcttccttcccaccaacag

SEQ ID NO: 175 - DNA, 21 bp, Artificial Sequence
  Synthetic polynucleotide
ttttttcccattaagattcct

SEQ ID NO: 176 - DNA, 30 bp, Artificial Sequence
  Synthetic polynucleotide
acgttggatgtccatggatgagaattcagc

SEQ ID NO: 177 - DNA, 30 bp, Artificial Sequence
  Synthetic polynucleotide
acgttggatggcacagctttcaccacaaac

SEQ ID NO: 178 - DNA, 21 bp, Artificial Sequence
  Synthetic polynucleotide
gaccacaaactcatggacttc

SEQ ID NO: 179 - DNA, 30 bp, Artificial Sequence
  Synthetic polynucleotide
acgttggatgtgtacaacttagagggcctg

SEQ ID NO: 180 - DNA, 30 bp, Artificial Sequence
  Synthetic polynucleotide
acgttggatgaagtggagagtttcagtgcc

SEQ ID NO: 181 - DNA, 22 bp, Artificial Sequence
  Synthetic polynucleotide
ttcagtgccagaatgaattttc

SEQ ID NO: 182 - DNA, 30 bp, Artificial Sequence; identical to SEQ ID NO: 126
  Synthetic polynucleotide

SEQ ID NO: 183 - DNA, 30 bp, Artificial Sequence; identical to SEQ ID NO: 125
  Synthetic polynucleotide

SEQ ID NO: 184 - DNA, 22 bp, Artificial Sequence
  Synthetic polynucleotide
ctgtggaaaaatttctttcaaa

SEQ ID NO: 185 - DNA, 30 bp, Artificial Sequence
  Synthetic polynucleotide
acgttggatgcagctgcactggtaatgatg

SEQ ID NO: 186 - DNA, 30 bp, Artificial Sequence
  Synthetic polynucleotide
acgttggatgggatataatggcagcagtgg

SEQ ID NO: 187 - DNA, 23 bp, Artificial Sequence
  Synthetic polynucleotide
ccctcagtggtccccaaaatact

SEQ ID NO: 188 - DNA, 30 bp, Artificial Sequence; identical to SEQ ID NO: 74
  Synthetic polynucleotide

SEQ ID NO: 189 - DNA, 30 bp, Artificial Sequence; identical to SEQ ID NO: 75
  Synthetic polynucleotide

SEQ ID NO: 190 - DNA, 24 bp, Artificial Sequence
  Synthetic polynucleotide
ccggagcttgttcaaattccaact

SEQ ID NO: 191 - DNA, 30 bp, Artificial Sequence
  Synthetic polynucleotide
acgttggatgcattggctggaaatgagtgc

SEQ ID NO: 192 - DNA, 30 bp, Artificial Sequence
  Synthetic polynucleotide
acgttggatggcagctctttactcagagca

SEQ ID NO: 193 - DNA, 24 bp, Artificial Sequence
  Synthetic polynucleotide
ggaggtaaaatcaccttgctgata

SEQ ID NO: 194 - DNA, 30 bp, Artificial Sequence
  Synthetic polynucleotide
acgttggatggagggttttcactctcccag

SEQ ID NO: 195 - DNA, 31 bp, Artificial Sequence
  Synthetic polynucleotide
acgttggatgcatttatctcttaattggtgg

SEQ ID NO: 196 - DNA, 25 bp, Artificial Sequence
  Synthetic polynucleotide
ttaatgtatactttatggttctagg

SEQ ID NO: 197 - DNA, 30 bp, Artificial Sequence
  Synthetic polynucleotide
acgttggatgcatgaagatttactgaagcg

SEQ ID NO: 198 - DNA, 30 bp, Artificial Sequence
  Synthetic polynucleotide
acgttggatgaccaaacactgcattctgcc

SEQ ID NO: 199 - DNA, 25 bp, Artificial Sequence
  Synthetic polynucleotide
gggatgagtgaatcatctgaagata

SEQ ID NO: 200 - DNA, 29 bp, Artificial Sequence
  Synthetic polynucleotide
acgttggatgtctccttgttacttacttg

SEQ ID NO: 201 - DNA, 30 bp, Artificial Sequence
  Synthetic polynucleotide
acgttggatggttgaaccttcttggcttgg

SEQ ID NO: 202 - DNA, 26 bp, Artificial Sequence
  Synthetic polynucleotide
tgtcctttatttccagataagacttc

SEQ ID NO: 203 - DNA, 30 bp, Artificial Sequence
  Synthetic polynucleotide
acgttggatggtagcaaccatgcattcaac

SEQ ID NO: 204 - DNA, 30 bp, Artificial Sequence
  Synthetic polynucleotide
acgttggatgctcaggtctttgacattttg

SEQ ID NO: 205 - DNA, 26 bp, Artificial Sequence
  Synthetic polynucleotide
gaagtcagctgaattagtctgtctga

SEQ ID NO: 206 - DNA, 29 bp, Artificial Sequence
  Synthetic polynucleotide
acgttggatggctgttattctgatcacac

SEQ ID NO: 207 - DNA, 30 bp, Artificial Sequence; identical to SEQ ID NO: 44
  Synthetic polynucleotide

SEQ ID NO: 208 - DNA, 26 bp, Artificial Sequence
  Synthetic polynucleotide
atctggatgattacttaaaaaatgtg

SEQ ID NO: 209 - DNA, 30 bp, Artificial Sequence
  Synthetic polynucleotide
acgttggatgtaacttgccatcagcggttc

SEQ ID NO: 210 - DNA, 30 bp, Artificial Sequence
  Synthetic polynucleotide
acgttggatggtggaggagaacattaaggc

SEQ ID NO: 211 - DNA, 27 bp, Artificial Sequence
  Synthetic polynucleotide
ccacttttccttttagcaaacaaaaaa

SEQ ID NO: 212 - DNA, 30 bp, Artificial Sequence
  Synthetic polynucleotide
acgttggatgtggacattgttcagcctgac

SEQ ID NO: 213 - DNA, 29 bp, Artificial Sequence
  Synthetic polynucleotide
acgttggatgatggctagtggcagctgtc

SEQ ID NO: 214 - DNA, 19 bp, Artificial Sequence
  Synthetic polynucleotide
ccagtttcctcgtcctctt

SEQ ID NO: 215 - DNA, 30 bp, Artificial Sequence
  Synthetic polynucleotide
acgttggatgtgtggtagagaaactggagc

SEQ ID NO: 216 - DNA, 30 bp, Artificial Sequence
  Synthetic polynucleotide
acgttggatgtcagcatatttaggcaaccc

SEQ ID NO: 217 - DNA, 18 bp, Artificial Sequence
  Synthetic polynucleotide
agctctttcttacccttc

SEQ ID NO: 218 - DNA, 30 bp, Artificial Sequence; identical to SEQ ID NO: 12
  Synthetic polynucleotide

SEQ ID NO: 219 - DNA, 30 bp, Artificial Sequence; identical to SEQ ID NO: 11
  Synthetic polynucleotide

SEQ ID NO: 220 - DNA, 18 bp, Artificial Sequence
  Synthetic polynucleotide
tgtctttccctccaggct

SEQ ID NO: 221 - DNA, 30 bp, Artificial Sequence
  Synthetic polynucleotide
acgttggatgctctgtgacacatgaagtgc

SEQ ID NO: 222 - DNA, 30 bp, Artificial Sequence
  Synthetic polynucleotide
acgttggatgaactgaaaaactctgtgggc

SEQ ID NO: 223 - DNA, 18 bp, Artificial Sequence
  Synthetic polynucleotide
agtgggctaatagactgc

SEQ ID NO: 224 - DNA, 30 bp, Artificial Sequence; identical to SEQ ID NO: 147
  Synthetic polynucleotide

SEQ ID NO: 225 - DNA, 30 bp, Artificial Sequence; identical to SEQ ID NO: 146
  Synthetic polynucleotide

SEQ ID NO: 226 - DNA, 19 bp, Artificial Sequence
  Synthetic polynucleotide
ccaatacattctgtttggg

SEQ ID NO: 227 - DNA, 30 bp, Artificial Sequence; identical to SEQ ID NO: 143
  Synthetic polynucleotide

SEQ ID NO: 228 - DNA, 30 bp, Artificial Sequence
  Synthetic polynucleotide
acgttggatggaatacctgggcagaagtag

SEQ ID NO: 229 - DNA, 19 bp, Artificial Sequence
  Synthetic polynucleotide
caaagtaggcctttcttga

SEQ ID NO: 230 - DNA, 30 bp, Artificial Sequence
  Synthetic polynucleotide
acgttggatgtgtcagcagcaaaacacaac

SEQ ID NO: 231 - DNA, 30 bp, Artificial Sequence
  Synthetic polynucleotide
acgttggatgttaccgagatgccgtattac

SEQ ID NO: 232 - DNA, 19 bp, Artificial Sequence
  Synthetic polynucleotide
ccttgctcaccaaatttgc

SEQ ID NO: 233 - DNA, 30 bp, Artificial Sequence
  Synthetic polynucleotide
acgttggatgtggaatgcaaagtgccacag

SEQ ID NO: 234 - DNA, 30 bp, Artificial Sequence; identical to SEQ ID NO: 105
  Synthetic polynucleotide

SEQ ID NO: 235 - DNA, 20 bp, Artificial Sequence
  Synthetic polynucleotide
ggtcttatttggataggcca

SEQ ID NO: 236 - DNA, 30 bp, Artificial Sequence
  Synthetic polynucleotide
acgttggatggttgtccaaaacaccctaag

SEQ ID NO: 237 - DNA, 30 bp, Artificial Sequence; identical to SEQ ID NO: 126
  Synthetic polynucleotide

SEQ ID NO: 238 - DNA, 21 bp, Artificial Sequence
  Synthetic polynucleotide
agaaatttttccacatctcta

SEQ ID NO: 239 - DNA, 30 bp, Artificial Sequence
  Synthetic polynucleotide
acgttggatgactgacaagtttacactggc

SEQ ID NO: 240 - DNA, 31 bp, Artificial Sequence
  Synthetic polynucleotide
acgttggatggcaaataagcaaacttttgag

SEQ ID NO: 241 - DNA, 22 bp, Artificial Sequence
  Synthetic polynucleotide
gtttttaactccagattgattc

SEQ ID NO: 242 - DNA, 30 bp, Artificial Sequence; identical to SEQ ID NO: 164
  Synthetic polynucleotide

SEQ ID NO: 243 - DNA, 30 bp, Artificial Sequence; identical to SEQ ID NO: 42
  Synthetic polynucleotide

SEQ ID NO: 244 - DNA, 22 bp, Artificial Sequence
  Synthetic polynucleotide
aaagatgctgatctgattatgg

SEQ ID NO: 245 - DNA, 30 bp, Artificial Sequence
  Synthetic polynucleotide
acgttggatgttatccccattctacagcag

SEQ ID NO: 246 - DNA, 30 bp, Artificial Sequence
  Synthetic polynucleotide
acgttggatgcacactgtatcccaatgctg

SEQ ID NO: 247 - DNA, 23 bp, Artificial Sequence
  Synthetic polynucleotide
cccttgcaaagattgctgactac

SEQ ID NO: 248 - DNA, 30 bp, Artificial Sequence
  Synthetic polynucleotide
acgttggatgctctccatgctgatgcattc

SEQ ID NO: 249 - DNA, 30 bp, Artificial Sequence
  Synthetic polynucleotide
acgttggatggcccactgcttaccattttg

SEQ ID NO: 250 - DNA, 23 bp, Artificial Sequence
  Synthetic polynucleotide
agaggagagttgacaatgcattc

SEQ ID NO: 251 - DNA, 30 bp, Artificial Sequence
  Synthetic polynucleotide
acgttggatgggattcttgcctgtcgtttg

SEQ ID NO: 252 - DNA, 30 bp, Artificial Sequence
  Synthetic polynucleotide
acgttggatgtcgctgcgtcataaaatggg

SEQ ID NO: 253 - DNA, 24 bp, Artificial Sequence
  Synthetic polynucleotide
ttctgagtactatagaattcctca

SEQ ID NO: 254 - DNA, 30 bp, Artificial Sequence
  Synthetic polynucleotide
acgttggatgattcacaaactggtcctagc

SEQ ID NO: 255 - DNA, 30 bp, Artificial Sequence
  Synthetic polynucleotide
acgttggatgcccctagttctccaaattta

SEQ ID NO: 256 - DNA, 26 bp, Artificial Sequence
  Synthetic polynucleotide
ccgaaaacttatattcaacataccct

SEQ ID NO: 257 - DNA, 30 bp, Artificial Sequence
  Synthetic polynucleotide
acgttggatggcctccttctatgacaatcc

SEQ ID NO: 258 - DNA, 30 bp, Artificial Sequence
  Synthetic polynucleotide
acgttggatgttctgttccagctaatgtgc

SEQ ID NO: 259 - DNA, 26 bp, Artificial Sequence
  Synthetic polynucleotide
gggcacttactgtggaagatttaaaa

SEQ ID NO: 260 - DNA, 30 bp, Artificial Sequence; identical to SEQ ID NO: 12
  Synthetic polynucleotide

SEQ ID NO: 261 - DNA, 30 bp, Artificial Sequence; identical to SEQ ID NO: 11
  Synthetic polynucleotide

SEQ ID NO: 262 - DNA, 17 bp, Artificial Sequence
  Synthetic polynucleotide
gtctttccctccaggct

SEQ ID NO: 263 - DNA, 30 bp, Artificial Sequence
  Synthetic polynucleotide
acgttggatgaggctaactccatcaccaac

SEQ ID NO: 264 - DNA, 30 bp, Artificial Sequence
  Synthetic polynucleotide
acgttggatgttgtggtgctttgccacctc

SEQ ID NO: 265 - DNA, 17 bp, Artificial Sequence
  Synthetic polynucleotide
gctggcagctgggattc

SEQ ID NO: 266 - DNA, 30 bp, Artificial Sequence
  Synthetic polynucleotide
acgttggatgcttgtctcttgtgactagaa

SEQ ID NO: 267 - DNA, 30 bp, Artificial Sequence
  Synthetic polynucleotide
acgttggatgagttccttcagtctcaaggg

SEQ ID NO: 268 - DNA, 18 bp, Artificial Sequence
  Synthetic polynucleotide
gtctcaagggggagcata

SEQ ID NO: 269 - DNA, 30 bp, Artificial Sequence; identical to SEQ ID NO: 147
  Synthetic polynucleotide

SEQ ID NO: 270 - DNA, 30 bp, Artificial Sequence; identical to SEQ ID NO: 146
  Synthetic polynucleotide

SEQ ID NO: 271 - DNA, 19 bp, Artificial Sequence
  Synthetic polynucleotide
ccaatacattctgtttggg

SEQ ID NO: 272 - DNA, 30 bp, Artificial Sequence; identical to SEQ ID NO: 75
  Synthetic polynucleotide

SEQ ID NO: 273 - DNA, 30 bp, Artificial Sequence; identical to SEQ ID NO: 74
  Synthetic polynucleotide

SEQ ID NO: 274 - DNA, 21 bp, Artificial Sequence
  Synthetic polynucleotide
cctgcctagaaatattatgtt

SEQ ID NO: 275 - DNA, 30 bp, Artificial Sequence
  Synthetic polynucleotide
acgttggatgaaaagacaccaacctgtgcc

SEQ ID NO: 276 - DNA, 30 bp, Artificial Sequence
  Synthetic polynucleotide
acgttggatgcagcactgcagttaagtgac

SEQ ID NO: 277 - DNA, 22 bp, Artificial Sequence
  Synthetic polynucleotide
aagttaagtgacttgtactttg

SEQ ID NO: 278 - DNA, 30 bp, Artificial Sequence; identical to SEQ ID NO: 38
  Synthetic polynucleotide

SEQ ID NO: 279 - DNA, 30 bp, Artificial Sequence; identical to SEQ ID NO: 245
  Synthetic polynucleotide

SEQ ID NO: 280 - DNA, 24 bp, Artificial Sequence
  Synthetic polynucleotide
cccattctacagcagtactgagca

SEQ ID NO: 281 - DNA, 30 bp, Artificial Sequence; identical to SEQ ID NO: 126
  Synthetic polynucleotide

SEQ ID NO: 282 - DNA, 30 bp, Artificial Sequence; identical to SEQ ID NO: 125
  Synthetic polynucleotide

SEQ ID NO: 283 - DNA, 25 bp, Artificial Sequence
  Synthetic polynucleotide
ccacggaaatttccaaagaactaca

SEQ ID NO: 284 - DNA, 30 bp, Artificial Sequence; identical to SEQ ID NO: 50
  Synthetic polynucleotide

SEQ ID NO: 285 - DNA, 30 bp, Artificial Sequence
  Synthetic polynucleotide
acgttggatggcacagccttttttaatcgc

SEQ ID NO: 286 - DNA, 26 bp, Artificial Sequence
  Synthetic polynucleotide
gccttttttaatcgctgttgaagaaa

SEQ ID NO: 287 - DNA, 30 bp, Artificial Sequence
  Synthetic polynucleotide
acgttggatggttgatgccatgaagccttg

SEQ ID NO: 288 - DNA, 29 bp, Artificial Sequence
  Synthetic polynucleotide
acgttggatgcacacagacacatgaatta

SEQ ID NO: 289 - DNA, 15 bp, Artificial Sequence
  Synthetic polynucleotide
aagccttggctcttc

SEQ ID NO: 290 - DNA, 30 bp, Artificial Sequence; identical to SEQ ID NO: 12
  Synthetic polynucleotide

SEQ ID NO: 291 - DNA, 30 bp, Artificial Sequence; identical to SEQ ID NO: 11
  Synthetic polynucleotide

SEQ ID NO: 292 - DNA, 16 bp, Artificial Sequence
  Synthetic polynucleotide
gccaacgagaatcaca

SEQ ID NO: 293 - DNA, 30 bp, Artificial Sequence
  Synthetic polynucleotide
acgttggatgtgctattgttggccacatcc

SEQ ID NO: 294 - DNA, 30 bp, Artificial Sequence
  Synthetic polynucleotide
acgttggatgagcattgggaaaggtgacag

SEQ ID NO: 295 - DNA, 18 bp, Artificial Sequence
  Synthetic polynucleotide
tcagcttgctcttaagga

SEQ ID NO: 296 - DNA, 30 bp, Artificial Sequence
  Synthetic polynucleotide
acgttggatgtcttctcctattggcaaagc

SEQ ID NO: 297 - DNA, 30 bp, Artificial Sequence
  Synthetic polynucleotide
acgttggatgggaaatttccaaagaactac

SEQ ID NO: 298 - DNA, 20 bp, Artificial Sequence
  Synthetic polynucleotide
aaagcaatctggaatttttc

SEQ ID NO: 299 - DNA, 30 bp, Artificial Sequence
  Synthetic polynucleotide
acgttggatgggagcaactgactgaatttg

SEQ ID NO: 300 - DNA, 30 bp, Artificial Sequence
  Synthetic polynucleotide
acgttggatggcacatgaagcacaatttcc

SEQ ID NO: 301 - DNA, 27 bp, Artificial Sequence
  Synthetic polynucleotide
acaaagattatatgatattgttaagtg

SEQ ID NO: 302 - DNA, 30 bp, Artificial Sequence; identical to SEQ ID NO: 96
  Synthetic polynucleotide

SEQ ID NO: 303 - DNA, 30 bp, Artificial Sequence
  Synthetic polynucleotide
acgttggatgggcacacatttggatgtttc

SEQ ID NO: 304 - DNA, 15 bp, Artificial Sequence
  Synthetic polynucleotide
actcatgcaggcttt

SEQ ID NO: 305 - DNA, 30 bp, Artificial Sequence
  Synthetic polynucleotide
acgttggatgccacaacagggctattttgc

SEQ ID NO: 306 - DNA, 30 bp, Artificial Sequence
  Synthetic polynucleotide
acgttggatgcagctttcagtgattccaac

SEQ ID NO: 307 - DNA, 17 bp, Artificial Sequence
  Synthetic polynucleotide
agtgtctaccaccactg

SEQ ID NO: 308 - DNA, 30 bp, Artificial Sequence; identical to SEQ ID NO: 117
  Synthetic polynucleotide

SEQ ID NO: 309 - DNA, 30 bp, Artificial Sequence; identical to SEQ ID NO: 116
  Synthetic polynucleotide

SEQ ID NO: 310 - DNA, 18 bp, Artificial Sequence
  Synthetic polynucleotide
aagccagaaatgctgtat

SEQ ID NO: 311 - DNA, 30 bp, Artificial Sequence
  Synthetic polynucleotide
acgttggatgattcaagaagtgagttgctg

SEQ ID NO: 312 - DNA, 30 bp, Artificial Sequence
  Synthetic polynucleotide
acgttggatgatcatacagtgtgtgatgac

SEQ ID NO: 313 - DNA, 19 bp, Artificial Sequence
  Synthetic polynucleotide
ccctttgctgtttgctcca

SEQ ID NO: 314 - DNA, 30 bp, Artificial Sequence
  Synthetic polynucleotide
acgttggatgtgctggtattgggctacaac

SEQ ID NO: 315 - DNA, 30 bp, Artificial Sequence
  Synthetic polynucleotide
acgttggatgcacatcattgccctgtttgg

SEQ ID NO: 316 - DNA, 19 bp, Artificial Sequence
  Synthetic polynucleotide
aagtggctacaaccggaaa

SEQ ID NO: 317 - DNA, 30 bp, Artificial Sequence
  Synthetic polynucleotide
acgttggatggtcttcattagatacttacc

SEQ ID NO: 318 - DNA, 30 bp, Artificial Sequence
  Synthetic polynucleotide
acgttggatgccaatgggattttggtcaag

SEQ ID NO: 319 - DNA, 20 bp, Artificial Sequence
  Synthetic polynucleotide
attagatacttacctctccc

SEQ ID NO: 320 - DNA, 30 bp, Artificial Sequence
  Synthetic polynucleotide
acgttggatggttacaaagcattaacgtgg

SEQ ID NO: 321 - DNA, 31 bp, Artificial Sequence
  Synthetic polynucleotide
acgttggatgctctaatatttatgagagtcc

SEQ ID NO: 322 - DNA, 20 bp, Artificial Sequence
  Synthetic polynucleotide
ggcatagaaagaacaagcac

SEQ ID NO: 323 - DNA, 31 bp, Artificial Sequence
  Synthetic polynucleotide
acgttggatggggatttcagacaatcttagc

SEQ ID NO: 324 - DNA, 30 bp, Artificial Sequence
  Synthetic polynucleotide
acgttggatgcaaatgaatgatgcaccagc

SEQ ID NO: 325 - DNA, 21 bp, Artificial Sequence
  Synthetic polynucleotide
ctcaaattgtcagcatctttt

SEQ ID NO: 326 - DNA, 30 bp, Artificial Sequence; identical to SEQ ID NO: 2
  Synthetic polynucleotide

SEQ ID NO: 327 - DNA, 30 bp, Artificial Sequence; identical to SEQ ID NO: 3
  Synthetic polynucleotide

SEQ ID NO: 328 - DNA, 21 bp, Artificial Sequence
  Synthetic polynucleotide
tcattgtaccttgctgtctat

SEQ ID NO: 329 - DNA, 30 bp, Artificial Sequence
  Synthetic polynucleotide
acgttggatgtttgcaaatgtaaggagggg

SEQ ID NO: 330 - DNA, 30 bp, Artificial Sequence
  Synthetic polynucleotide
acgttggatgaggatctgctggtgttcacg

SEQ ID NO: 331 - DNA, 22 bp, Artificial Sequence
  Synthetic polynucleotide
gtttattttcaagtgattaccg

SEQ ID NO: 332 - DNA, 29 bp, Artificial Sequence
  Synthetic polynucleotide
acgttggatggcatcaggaaaatgtacaa

SEQ ID NO: 333 - DNA, 29 bp, Artificial Sequence
  Synthetic polynucleotide
acgttggatgaacagcgagtattcttttg

SEQ ID NO: 334 - DNA, 23 bp, Artificial Sequence
  Synthetic polynucleotide
ccttttaatccacatttctgaat

SEQ ID NO: 335 - DNA, 30 bp, Artificial Sequence
  Synthetic polynucleotide
acgttggatgctattctacctccagttgtc

SEQ ID NO: 336 - DNA, 30 bp, Artificial Sequence
  Synthetic polynucleotide
acgttggatgaagttgccagaggaaatgtc

SEQ ID NO: 337 - DNA, 23 bp, Artificial Sequence
  Synthetic polynucleotide
ctccagttgtcaaaatgtcatag

SEQ ID NO: 338 - DNA, 30 bp, Artificial Sequence
  Synthetic polynucleotide
acgttggatgttctgatcacacgctctctc

SEQ ID NO: 339 - DNA, 30 bp, Artificial Sequence; identical to SEQ ID NO: 44
  Synthetic polynucleotide

SEQ ID NO: 340 - DNA, 24 bp, Artificial Sequence
  Synthetic polynucleotide
tctatctctagcatcacatttttt

SEQ ID NO: 341 - DNA, 30 bp, Artificial Sequence
  Synthetic polynucleotide
acgttggatgctgttgatcgtcttggactc

SEQ ID NO: 342 - DNA, 30 bp, Artificial Sequence
  Synthetic polynucleotide
acgttggatgagggagaatgagttgaagtg

SEQ ID NO: 343 - DNA, 24 bp, Artificial Sequence
  Synthetic polynucleotide
ggtgtactcctatatgagagtcgc

SEQ ID NO: 344 - DNA, 30 bp, Artificial Sequence; identical to SEQ ID NO: 23
  Synthetic polynucleotide

SEQ ID NO: 345 - DNA, 30 bp, Artificial Sequence; identical to SEQ ID NO: 24
  Synthetic polynucleotide

SEQ ID NO: 346 - DNA, 24 bp, Artificial Sequence
  Synthetic polynucleotide
ggtgagaaaactgaaaaactctgt

SEQ ID NO: 347 - DNA, 29 bp, Artificial Sequence
  Synthetic polynucleotide
acgttggatggttatctcttcccacaatc

SEQ ID NO: 348 - DNA, 30 bp, Artificial Sequence
  Synthetic polynucleotide
acgttggatgggcgcttcgagctattttac

SEQ ID NO: 349 - DNA, 26 bp, Artificial Sequence
  Synthetic polynucleotide
cccaacaatcaactatgtaacttacc

SEQ ID NO: 350 - DNA, 30 bp, Artificial Sequence
  Synthetic polynucleotide
acgttggatgcaagagaatgatgatgaggg

SEQ ID NO: 351 - DNA, 30 bp, Artificial Sequence
  Synthetic polynucleotide
acgttggatgctgcacgtgcttgttctttc

SEQ ID NO: 352 - DNA, 16 bp, Artificial Sequence
  Synthetic polynucleotide
cgttttggctggaagc

SEQ ID NO: 353 - DNA, 30 bp, Artificial Sequence
  Synthetic polynucleotide
acgttggatgaccttgctgtctatgacctc

SEQ ID NO: 354 - DNA, 29 bp, Artificial Sequence
  Synthetic polynucleotide
acgttggatggaacaaatcacctttatat

SEQ ID NO: 355 - DNA, 16 bp, Artificial Sequence
  Synthetic polynucleotide
aggctgaagttgatgc

SEQ ID NO: 356 - DNA, 30 bp, Artificial Sequence
  Synthetic polynucleotide
acgttggatggaagtccatgagtttgtggt

SEQ ID NO: 357 - DNA, 30 bp, Artificial Sequence
  Synthetic polynucleotide
acgttggatgctgcaatgctgcattctctg

SEQ ID NO: 358 - DNA, 18 bp, Artificial Sequence
  Synthetic polynucleotide
tgagtttgtggtgaaagc

SEQ ID NO: 359 - DNA, 30 bp, Artificial Sequence; identical to SEQ ID NO: 306
  Synthetic polynucleotide

SEQ ID NO: 360 - DNA, 30 bp, Artificial Sequence; identical to SEQ ID NO: 305
  Synthetic polynucleotide

SEQ ID NO: 361 - DNA, 20 bp, Artificial Sequence
  Synthetic polynucleotide
cagtgattccaacatcataa

SEQ ID NO: 362 - DNA, 29 bp, Artificial Sequence
  Synthetic polynucleotide
acgttggatgtatgactcgaatctttcag

SEQ ID NO: 363 - DNA, 29 bp, Artificial Sequence
  Synthetic polynucleotide
acgttggatgcccttagggtgttttggac

SEQ ID NO: 364 - DNA, 21 bp, Artificial Sequence
  Synthetic polynucleotide
gaatctttcagattttgacag

SEQ ID NO: 365 - DNA, 30 bp, Artificial Sequence
  Synthetic polynucleotide
acgttggatgcctgttgatccttttgttcc

SEQ ID NO: 366 - DNA, 30 bp, Artificial Sequence
  Synthetic polynucleotide
acgttggatgaagctgctggtgcatcattc

SEQ ID NO: 367 - DNA, 21 bp, Artificial Sequence
  Synthetic polynucleotide
ggaagacatttgatggaatat

SEQ ID NO: 368 - DNA, 30 bp, Artificial Sequence
  Synthetic polynucleotide
acgttggatggaaacatccaaatgtgtgcc

SEQ ID NO: 369 - DNA, 30 bp, Artificial Sequence
  Synthetic polynucleotide
acgttggatgagagggttttgtgtctttcc

SEQ ID NO: 370 - DNA, 21 bp, Artificial Sequence
  Synthetic polynucleotide
ggggccaggggaagaagaagc

SEQ ID NO: 371 - DNA, 35 bp, Artificial Sequence
  Synthetic polynucleotide at Figure 3B
aaagtgccacagttggcgtagatgtgaaagactgg

SEQ ID NO: 372 - protein, 2527 aa, Homo sapiens
  VARIANT (1)..(2527): Variants as listed in Table 1
  VARIANT (10)..(10): E or K
  VARIANT (50)..(50): R or H
  VARIANT (52)..(52): S or F
  VARIANT (75)..(75): A
  VARIANT (119)..(119): L or P
  VARIANT (122)..(122): L
  VARIANT (153)..(153): L
  VARIANT (182)..(182): K
  VARIANT (211)..(211): A or V
  VARIANT (228)..(228): C or S
  VARIANT (238)..(238): N or I
  VARIANT (275)..(275): H
  VARIANT (289)..(289): N
  VARIANT (298)..(298): A
  VARIANT (312)..(312): A
  VARIANT (334)..(334): E or K
  VARIANT (363)..(363): N or S
  VARIANT (366)..(366): V or M
  VARIANT (419)..(419): A or V
  VARIANT (461)..(461): S
  VARIANT (488)..(488): L
  VARIANT (521)..(521): R or G
  VARIANT (544)..(544): K or E
  VARIANT (551)..(551): N or K
  VARIANT (558)..(558): G
  VARIANT (663)..(663): S or P
  VARIANT (674)..(674): V
  VARIANT (712)..(712): M or V
  VARIANT (716)..(716): A or V
  VARIANT (723)..(723): I or V
  VARIANT (755)..(755): P or L
  VARIANT (793)..(793): R or M
  VARIANT (810)..(810): I or V
  VARIANT (827)..(827): S
  VARIANT (871)..(871): K or E
  VARIANT (923)..(923): Q or H
  VARIANT (930)..(930): Q or R
  VARIANT (944)..(944): D or Y
  VARIANT (953)..(953): L
  VARIANT (973)..(973): S or N
  VARIANT (1006)..(1006): I or M
  VARIANT (1007)..(1007): S
  VARIANT (1067)..(1067): R or Q
  VARIANT (1096)..(1096): S or C
  VARIANT (1111)..(1111): Q or H
  VARIANT (1114)..(1114): L
  VARIANT (1122)..(1122): I or V
  VARIANT (1151)..(1151): A or T
  VARIANT (1165)..(1165): L or P
  VARIANT (1192)..(1192): I or V
  VARIANT (1216)..(1216): H or R
  VARIANT (1228)..(1228): S, T or I
  VARIANT (1262)..(1262): P or A
  VARIANT (1320)..(1320): R or S
  VARIANT (1325)..(1325): R or Q
  VARIANT (1371)..(1371): I or V
  VARIANT (1375)..(1375): D or E
  VARIANT (1398)..(1398): R or H
  VARIANT (1410)..(1410): T or M
  VARIANT (1420)..(1420): D or N
  VARIANT (1423)..(1423): K
  VARIANT (1430)..(1430): A
  VARIANT (1437)..(1437): N or H
  VARIANT (1441)..(1441): C, G, H, or R
  VARIANT (1442)..(1442): A or P
  VARIANT (1446)..(1446): P or L
  VARIANT (1450)..(1450): V or I
  VARIANT (1468)..(1468): K or E
  VARIANT (1483)..(1483): R or Q
  VARIANT (1514)..(1514): R or Q
  VARIANT (1542)..(1542): P or S
  VARIANT (1556)..(1556): L or I
  VARIANT (1598)..(1598): V or E
  VARIANT (1613)..(1613): V or A
  VARIANT (1624)..(1624): G
  VARIANT (1628)..(1628): R or P
  VARIANT (1637)..(1637): K
  VARIANT (1646)..(1646): M or T
  VARIANT (1647)..(1647): S or T
  VARIANT (1653)..(1653): L
  VARIANT (1699)..(1699): Y or C
  VARIANT (1721)..(1721): S
  VARIANT (1725)..(1725): R or STOP
  VARIANT (1728)..(1728): R, L or H
  VARIANT (1795)..(1795): L or F
  VARIANT (1819)..(1819): G
  VARIANT (1869)..(1869): M, V or T
  VARIANT (1870)..(1870): L or F
  VARIANT (1874)..(1874): E or STOP
  VARIANT (1941)..(1941): R or H
  VARIANT (2006)..(2006): Y or H
  VARIANT (2012)..(2012): I or T
  VARIANT (2019)..(2019): G or S
  VARIANT (2020)..(2020): I or T
  VARIANT (2031)..(2031): T or S
  VARIANT (2063)..(2063): L or STOP
  VARIANT (2081)..(2081): N or D
  VARIANT (2108)..(2108): E
  VARIANT (2119)..(2119): P or L
  VARIANT (2141)..(2141): T or M
  VARIANT (2170)..(2170): G
  VARIANT (2189)..(2189): Y or C
  VARIANT (2261)..(2261): N or I
  VARIANT (2356)..(2356): T or I
  VARIANT (2385)..(2385): R or G
  VARIANT (2390)..(2390): V or M
  VARIANT (2395)..(2395): E or K
  VARIANT (2397)..(2397): M or T
  VARIANT (2408)..(2408): M or I
  VARIANT (2466)..(2466): L or H
  VARIANT (2479)..(2479): N or D
MASGSCQGCXEDEETLKKLIVRLNNVQEGKQIETLVQILEDLLVFTYSEXAXKLFQGKNIHVPLLIVLDSYMRVXSVQQVGWSLLCKLIEVCPGTMQSLMGPQDVGNDWEVLGVHQLIXKMXTVHNASVNLSVIGLKTLDLLLTSGKITLLIXDEESDIFMLIFDAMHSFPANDEVQKLGCXALHVLFERVSEEQLTEFVENKDYMILLSXLTNFKDEEEIVLHVLHXLHSLAIPCNXVEVLMSGNVRCYNIVVEAMKAFPMSERIQEVSCCLLXRLTLGNFFNILVLXEVHEFVVKXVQQYPENAALQISXLSCLALLTETIFLNQDLEEKNXNQENDDEGEEDKLFWLEACYKALTWHRKXKHXQEAACWALNNLLMYQNSLHEKIGDEDGHFPAHREVMLSMLMHSSSKEVFQASXNALSTLLEQNVNFRKILLSKGIHLNVLELMQKHIHSPEVAEXGCKMLNHLFEGSNTSLDIMAAVVPKIXTVMKRHETSLPVQLEALRAILHFIVPGMPEESXEDTEFHHKLNMVKKQCFKNDIHXLVLAALXRFIGNPXIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFXKLLVHHSFDLXIFHQMSSNIMEQKDQQFLNLCCKCFAKVAMDDYLKNVXLERXCDQNNSXMVECLLLLGADANQAKEGSSLICQVCEKESSXKLVELLLNSGSREQDVRKALTISIGKGDSQIISLLLRXLALDVANNSICLGGFCXGKVEPSWLGPLFPDKTXNLRKQTNIASTLARMVIRYQMKSAVEEGTASGSDGNFSEDVLSXFDEWTFIPDSSMDSVFAQSDDLDSEGSEGSFLVKKKSNSISVGEFYRDAVLXRCSPNLXRHSNSLGPIFDHEXLLKRKRKIXSSDDSLRSSKLQSHMRHSDXISSLASEREYITSLDLSANELRDIDALSQKCCXXVHLEHLEKLELHQNALTSFPQQLCETLKSLTHLDLHSNKFTSFPSYLLKMSCIANLDVSXNDIGPSVVLDPTVKCPTLKQFNLSYNQLXFVPENLTDVVEKLEXLIXEGNKISGXCSPLRLKELKILNLSKNHISSLSENFLEXCPKVESFSARMNFXAAMPFLPPSMTILKLSQNKFSCIPEAXLNLPHLRSLDMSSNDIQYLPGPAXWKSLNLRELLFXHNQISILDLSEKAYLWSRVEKLHLSHNKLKEIPXEIGCLENLTSLDVSYNLELRSFPNEMGKLSKIWDLPLDELHLNFDFKHIGCKAKDIIXFLQQXLKKAVPYNRMKLMIVGNTGSGKTTLLQQLMKTKKSDLGMQSATVGXDVKXWPIQIRDKRKRDLVLNVWDFAGXEEFYSTHPHFMXQRALYLAVYXLSXGQAEVDXMKPWLFXIKAXXSSSXVILXGTHLDVSDEKQRKACMSXITKELLNKRGFPAIXDYHFVNATEESDALAKLRKTIINESLNFKIXDQLVVGQLIPDCYVELEKIILSERKNVXIEFPVIDRKRLLQXVRENQLQLDENELPHAVHFLNESGVLLHFQDPALQLSDLYFXEPKWLCKIMAQILTXKVEGCPKHPKXIISXRDVEKFLSXKRKFPKNYXXQYFKLXEKFQIALPIGEEYLLVPSSLSDHRPVIELPHCENSEIIIRLYEMPXFPMGFWSRLINRLLEISPYMLXGREXALXPNRMYWRQGIYLNWSPEAYCLVGSEVLDNHPESFLKITVPSCRKGCILLGQVVDHIDSLMEEWFPGXLEIDICGEGETLLKKWALYSFNDXEEHQKILLDDLMKKAEEGDLLVNPDQPRLTIPISQIAPDLILADLPRNIXXNNDXLEFEQAPEFLLGDGSFGSVYRAAYEGEEVAVKIFNKHTSLRLLRQELVVLCHLHHPSLISLLAAGIXPRMLVMELASKGSLDRLLQQDKASLTRTLQHRIALHVADGLRYLHSAMIIYRDLKPHNVLLFTLXPNAAIXAKIADYXXAQYCCRMGIKXSEGTPGFRAPEVARGNVIYNQQADVYSFGLLXYDILTTGGRIVEGLKFPXEFDELEIQGKLPDPVKEYGCAPWPMVXKLIKQCLKENXQERPTSAQVFDILNSAELVCLXRRILLPKNVIVECMVATHHNSRNASIWLXCGHTDRGQLSFLDLNTEGXTSEEVADSRILCLALVHLPVEKESWIVSGTQSGTLLVINTEDGKKRHTLEKMTDSVTCLYCNSFSKQSKQKXFLLVGTADGKLAIFEDKTVKLKGAAPLKILNIGNVSTPLMCLSESTNSTERNVMWGGCGTKIFSFSNDFTIQKLIETRTSQLFSYAAFSDSNIIXVVVDTALYIAKQNSPVVEVWDKKTEKLCXLIDCXHFLRXVXVKENKESKHKXSYSGRVKTLCLQKNTALWIGTGGGHILLLDLSTRRLIRVIYNFCNSVRVMMTAQLGSXKNVMLVLGYNRKXTEGTQKQKEIQSCLTVWDINLPHEVQNLEKHIEVRKELAEKMRRTSVE

(19) United States
(12) Patent Application Publication
(10) Pub. No.: US 2018/0179594 A1
(43) Pub. Date: Jun. 28, 2018

(54) MULTIPLE SYSTEM ATROPHY AND THE TREATMENT THEREOF

(71) Applicant: Parkinson's Institute, Sunnyvale, CA (US)

(72) Inventor: Birgitt SCHÜLE, Portola Valley, CA (US)

(21) Appl. No.: 15/795,956

(22) Filed: Oct. 27, 2017

Related U.S. Application Data
(60) Provisional application No. 62/415,211, filed on Oct. 31, 2016.

(51) Int. Cl. C12Q 1/6883

(52) U.S. Cl. CPC C12Q 1/6883; C12Q 2600/118; C12Q 2600/156

(57) ABSTRACT

Provided herein are methods, kits, and devices related to genetic variations of neurological disorders. For example, methods, kits, and devices for using such genetic variations to assess susceptibility of developing Multiple System Atrophy.

### CROSS REFERENCE

This application claims priority to U.S. Provisional Application No. 62/415,211, filed on Oct. 31, 2016, which is herein incorporated by reference in its entirety.

### BACKGROUND

Multiple system atrophy (MSA) is a rare rapidly progressing neurodegenerative disorder characterized by a combination of symptoms that affect both the autonomic nervous system and central nervous system. MSA symptoms reflect the progressive loss of function and death of nerve cells and supporting glia cells in the brain and spinal cord, and can range from fainting spells to tremor, rigidity, and loss of muscle coordination. These symptoms are often difficult to distinguish from the symptoms of Parkinson's disease. A distinguishing neuropathological feature of MSA is the accumulation of the protein alpha-synuclein in glia, whereas, in Parkinson's disease, alpha-synuclein typically accumulates in nerve cells.

### BRIEF SUMMARY

Provided herein are methods, kits, and devices for assessing neurodegenerative disorders in subjects.

In one aspect, disclosed herein is a method. The method can comprise assessing multiple system atrophy in a subject. The method can comprise obtaining a sample from a subject, assaying a sample for a LRRK2 genetic variation, wherein an LRRK2 genetic variation may not cause an LRRK2 mutation at positon 12020 and assessing multiple system atrophy in a subject. In some embodiments, an LRRK2 genetic variation may not cause an LRRK2 mutation at position L153, N551, 1723, L953, R1398, K1423, R1514, P1542, G1624, K1637, M1646, S1647, G1819, N2081, E2108, G2385, 12020, or M2397. In some embodiments, an LRRK2 genetic variation may not cause an LRRK2 mutation at position 1723, R1441, R1628, M1869, G2019, T1699, 12012, or T2356. A LRRK2 genetic variation can cause an LRRK2 mutation at position I1371. A LRRK2 mutation at position I1371 can comprise an I1371V mutation. A LRRK2 genetic variation can comprise one or more point mutations, polymorphisms, translocations, insertions, deletions, amplifications, inversions, microsatellites, interstitial deletions, copy number variations (CNVs), or any combination thereof. A sample can be a biological sample. A biological sample can comprise a nucleic acid. Assaying can comprise purifying a nucleic acid from a biological sample. Assaying can comprise amplifying at least one nucleotide sequence. Assaying can comprise a microarray analysis of a nucleic acid. A biological sample can be collected from blood, saliva, urine, serum, tears, skin, tissue, or hair. A subject can be a human. A subject can be more than 30 years old, more than 40 years old, more than 50 years old, more than 60 years old, or more than 70 years old. A subject can be symptomatic. A subject can be asymptomatic. In some embodiments, a subject can have a symptom of multiple system atrophy. In some embodiments, a subject can have at least two symptoms of multiple system atrophy. A symptom can comprise unilateral or bilateral onset, tremor at rest, progression in time, asymmetry or symmetry of motor symptoms, response to levodopa, dyskinesias, problems learning, accumulation of alpha-synuclein protein in the brain in the form of Lewy bodies, glial cytoplasmic inclusions, neuronal cytoplasmic inclusions or other types of depositions of alpha-synuclein, dementia, neurofibrillary tangles, tremor, rigidity, slowness of movement, postural instability, pill-rolling, bradykinesia, difficulties planning a movement, difficulties initiating a movement, difficulties executing a movement, difficulties performing sequential movements, difficulties performing simultaneous movements, ratchet rigidity, joint pain, reduced ability to move, impaired balance, frequently falling, gait disturbances, posture disturbances, festination, speech disturbances, swallowing disturbances, voice disorders, mask-like facial expression, small handwriting, executive dysfunction, planning problems, cognitive flexibility problems, abstract thinking problems, rule acquisition problems, initiating appropriate action problems, inhibiting inappropriate action problems, problems selecting relevant sensory information, fluctuation in attention, slowed cognitive speed, reduced memory, problems recalling learned information, visuospatial difficulties, depression, apathy, anxiety, impulse control behavior problems, craving, binge eating, hypersexuality, pathological gambling, hallucinations, delusions, daytime drowsiness, disturbances in REM sleep, insomnia, orthostatic hypotension, oily skin, excessive sweating, urinary incontinence, altered sexual function, constipation, gastric dysmotility, decreased blink rate, dry eyes, deficient ocular pursuit, saccadic movements, difficulties in directing gaze upward, blurred vision, double vision, impaired sense of smell, sensation of pain, paresthesia, reduced activity of dopamine-secreting cells, dysarthria, dysphagia, decreased production of perspiration, decreased production of tears, decreased production of saliva, impaired control of body temperature, impotence, or a combination thereof. Assaying can comprise analyzing whole genome or whole exome of a subject. In some embodiments, an assaying can comprise detecting a presence or absence of an LRRK2 genetic variation. Detecting a presence or an absence of an LRRK2 genetic variation can comprise detecting nucleic acid information. In some embodiments, nucleic acid information can be detected by at least one of PCR, sequencing, northern blot, immunohistochemistry, or any combination thereof. Sequencing can comprise a high-throughput sequencing method. A high-throughput sequencing method can comprise Massively Parallel Signature Sequencing (MPSS), polony sequencing, 454 pyrosequencing, Illumina sequencing, SOLiD™ sequencing, ion semiconductor sequencing, DNA nanoball sequencing, HeliScope™ single molecule sequencing, single molecule real time (SMRT™) sequencing, RNAP sequencing, Nanopore DNA sequencing, sequencing by hybridization, or microfluidic Sanger sequencing. The method can further comprise detecting multiple system atrophy in a subject if an LRRK2 genetic variation is present. In some embodiments, a detecting multiple system atrophy or grounds for a suspicion that a subject may have multiple system atrophy can be based on an assessment by a medical doctor, a psychologist, a neurologist, a psychiatrist, or other professional who screens subjects for multiple system atrophy. An assessment can comprise an evaluation of a subject's motor skills, autonomic function, neuropsychiatry, mood, cognition, behavior, thoughts, ability to sense, past medical history, or a combination thereof. In some embodiments, an evaluation can be performed by observation, a questionnaire, a checklist, a test, or a combination thereof. The method can further comprise identifying a subject as having an increased risk of developing multiple system atrophy if an LRRK2 genetic variation is detected. The method can further comprise administering a treatment to a subject. A treatment can comprise a pharmaceutical composition comprising an LRRK2 inhibitor. A LRRK2 inhibitor can comprise a small molecule, siRNA, or an antibody. A LRRK2 inhibitor can comprise PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, KN-62, KN-93, ML-7, ML-9, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, or K252a. In some embodiments, a treatment can ameliorate a symptom of multiple system atrophy in a subject. In some embodiments, disclosed herein are devices. A device can comprise a memory that stores executable instructions; and a processor that executes executable instructions to perform the method.

Disclosed herein are methods. The method can comprise obtaining a sample from a subject, wherein a subject is suspected of having multiple system atrophy. The method can comprise assaying a sample for an LRRK2 genetic variation, wherein an LRRK2 genetic variation may not cause an LRRK2 mutation at position L153, N551, 1723, L953, R1398, K1423, R1514, P1542, G1624, K1637, M1646, S1647, G1819, N2081, E2108, G2385, 12020, or M2397. In some embodiments, an LRRK2 genetic variation may not cause an LRRK2 mutation at position 1723, R1441, R1628, M1869, G2019, T1699, 12012 or T2356. A LRRK2 genetic variation can cause an LRRK2 mutation at position I1371. A LRRK2 mutation at position I1371 can comprise an I1371V mutation. A LRRK2 genetic variation can comprise one or more point mutations, polymorphisms, translocations, insertions, deletions, amplifications, inversions, microsatellites, interstitial deletions, copy number variations (CNVs), or any combination thereof. In some embodiments a sample can be a biological sample. A biological sample can comprise a nucleic acid. Assaying can comprise purifying a nucleic acid from a biological sample. Assaying can comprise amplifying at least one nucleotide sequence. Assaying can comprise a microarray analysis of a nucleic acid. In some embodiments, a biological sample can be collected from blood, saliva, urine, serum, tears, skin, tissue, or hair. In some embodiments, a subject can be a human. In some embodiments, a subject can be more than 30 years old, more than 40 years old, more than 50 years old, more than 60 years old, or more than 70 years old. In some embodiments, a subject can be symptomatic. In some embodiments, a subject can be asymptomatic. In some embodiments, a subject can have a symptom of multiple system atrophy. In some embodiments, a subject can have at least two symptoms of multiple system atrophy. A symptom can comprise unilateral or bilateral onset, tremor at rest, progression in time, asymmetry or symmetry of motor symptoms, response to levodopa, dyskinesias, problems learning, accumulation of alpha-synuclein protein in the brain in the form of Lewy bodies, glial cytoplasmic inclusions, neuronal cytoplasmic inclusions or other types of depositions of alpha-synuclein, dementia, neurofibrillary tangles, tremor, rigidity, slowness of movement, postural instability, pill-rolling, bradykinesia, difficulties planning a movement, difficulties initiating a movement, difficulties executing a movement, difficulties performing sequential movements, difficulties performing simultaneous movements, ratchet rigidity, joint pain, reduced ability to move, impaired balance, frequently falling, gait disturbances, posture disturbances, festination, speech disturbances, swallowing disturbances, voice disorders, mask-like face expression, small handwriting, executive dysfunction, planning problems, cognitive flexibility problems, abstract thinking problems, rule acquisition problems, initiating appropriate action problems, inhibiting inappropriate action problems, problems selecting relevant sensory information, fluctuation in attention, slowed cognitive speed, reduced memory, problems recalling learned information, visuospatial difficulties, depression, apathy, anxiety, impulse control behavior problems, craving, binge eating, hypersexuality, pathological gambling, hallucinations, delusions, daytime drowsiness, disturbances in REM sleep, insomnia, orthostatic hypotension, oily skin, excessive sweating, urinary incontinence, altered sexual function, constipation, gastric dysmotility, decreased blink rate, dry eyes, deficient ocular pursuit, saccadic movements, difficulties in directing gaze upward, blurred vision, double vision, impaired sense of smell, sensation of pain, paresthesia, reduced activity of dopamine-secreting cells, dysarthria, dysphagia, decrease production of perspiration, decrease production of tears, decrease production of saliva, impaired control of body temperature, impotence, or a combination thereof. Assaying can comprise analyzing the whole genome or whole exome of a subject. Assaying can comprise detecting a presence or absence of an LRRK2 genetic variation. Detecting a presence or an absence of an LRRK2 genetic variation can comprise detecting nucleic acid information. In some embodiments, nucleic acid information can be detected by at least one of PCR, sequencing, northern blot, immunohistochemistry, or any combination thereof. Sequencing can comprise a high-throughput sequencing method. A high throughput sequencing method can comprise Massively Parallel Signature Sequencing (MPSS), polony sequencing, 454 pyrosequencing, Illumina sequencing, SOLiD™ sequencing, ion semiconductor sequencing, DNA nanoball sequencing, HeliScope™ single molecule sequencing, single molecule real time (SMRT™) sequencing, RNAP sequencing, Nanopore DNA sequencing, sequencing by hybridization, or microfluidic Sanger sequencing. The method can further comprise detecting multiple system atrophy in a subject if an LRRK2 genetic variation is present. In some embodiments, a detecting multiple system atrophy or grounds for a suspicion that a subject may have multiple system atrophy can be based on an assessment by a medical doctor, a psychologist, a neurologist, a psychiatrist, or other professional who screens subjects for multiple system atrophy. An assessment can comprise an evaluation of a subject's motor skills, autonomic function, neuropsychiatry, mood, cognition, behavior, thoughts, ability to sense, past medical history, or a combination thereof. In some embodiments, an evaluation can be performed by observation, a questionnaire, a checklist, a test, or a combination thereof. The method can further comprise identifying a subject as having an increased risk of developing multiple system atrophy if an LRRK2 genetic variation is detected. The method can further comprise administering a treatment to a subject. A treatment can comprise a pharmaceutical composition comprising an LRRK2 inhibitor. A LRRK2 inhibitor can comprise a small molecule, siRNA, or an antibody. A LRRK2 inhibitor can comprise PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, KN-62, KN-93, ML-7, ML-9, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, or K252a. In some embodiments, a treatment can ameliorates a symptom of multiple system atrophy in a subject. In some embodiments, disclosed herein are devices. A device can comprise a memory that stores executable instructions; and a processor that executes executable instructions to perform the method.

Disclosed herein is a method. The method can be directed to treating or preventing multiple system atrophy in a subject. The method can comprise administering a therapeutically effective amount of an LRRK2 inhibitor, wherein a subject has an LRRK2 genetic variation, wherein an LRRK2 genetic variation may not cause an LRRK2 mutation at position N551, 1723, R1398, R1441, R1514, P1542, R1628, M1646, S1647, M1869, G2019, G2385, or T2397. A LRRK2 genetic variation can cause an LRRK2 mutation at position I1371. A LRRK2 mutation at position I1371 can comprise an I1371V mutation. A genetic variation can comprise one or more point mutations, polymorphisms, translocations, insertions, deletions, amplifications, inversions, microsatellites, interstitial deletions, copy number variations (CNVs), or any combination thereof. In some embodiments, the method treats multiple system atrophy. In some embodiments, the subject can be suffering from multiple system atrophy. In some embodiments, the method can prevent multiple system atrophy. In some embodiments, the subject may be at risk of developing multiple system atrophy. In some embodiments, the method can ameliorate a symptom of multiple system atrophy in a subject. In some embodiments, a subject can be a human. In some embodiments, a subject can be more than 30 years old, more than 40 years old, more than 50 years old, more than 60 years old, or more than 70 years old. In some embodiments, a subject can have a symptom of multiple system atrophy. A symptom of multiple system atrophy can comprise unilateral or bilateral onset, tremor at rest, progression in time, asymmetry or symmetry of motor symptoms, response to levodopa, dyskinesias, problems learning, accumulation of alpha-synuclein protein in the brain in the form of Lewy bodies, glial cytoplasmic inclusions, neuronal cytoplasmic inclusions or other types of depositions of alpha-synuclein, dementia, neurofibrillary tangles, tremor, rigidity, slowness of movement, postural instability, pill-rolling, bradykinesia, difficulties planning a movement, difficulties initiating a movement, difficulties executing a movement, difficulties performing sequential movements, difficulties performing simultaneous movements, ratchet rigidity, joint pain, reduced ability to move, impaired balance, frequently falling, gait disturbances, posture disturbances, festination, speech disturbances, swallowing disturbances, voice disorders, mask-like facial expression, small handwriting, executive dysfunction, planning problems, cognitive flexibility problems, abstract thinking problems, rule acquisition problems, initiating appropriate action problems, inhibiting inappropriate action problems, problems selecting relevant sensory information, fluctuation in attention, slowed cognitive speed, reduced memory, problems recalling learned information, visuospatial difficulties, depression, apathy, anxiety, impulse control behavior problems, craving, binge eating, hypersexuality, pathological gambling, hallucinations, delusions, daytime drowsiness, disturbances in REM sleep, insomnia, orthostatic hypotension, oily skin, excessive sweating, urinary incontinence, altered sexual function, constipation, gastric dysmotility, decreased blink rate, dry eyes, deficient ocular pursuit, saccadic movements, difficulties in directing gaze upward, blurred vision, double vision, impaired sense of smell, sensation of pain, paresthesia, reduced activity of dopamine-secreting cells, dysarthria, dysphagia, decreased production of perspiration, decreased production of tears, decreased production of saliva, impaired control of body temperature, impotence, or a combination thereof. In some embodiments, a therapeutically effective amount of an LRRK2 inhibitor can be administered orally, intraperitoneally, buccally, intravenously, parenterally, rectally, intradermally, transdermally, pulmonary, intracranially, nasally, topically, or by inhalation spray. In some embodiments, a therapeutically effective amount of an LRRK2 inhibitor can be at least 1 μg of an LRRK2 inhibitor per kg of a subject. In some embodiments, a therapeutically effective amount of an LRRK2 inhibitor can be less than 1000 mg of an LRRK2 inhibitor per kg of a subject. In some embodiments, a therapeutically effective amount of an LRRK2 inhibitor can be in a tablet, capsule, caplet, gel cap, powder, or solution dosage form. In some embodiments, a tablet, capsule, caplet, gel cap, powder, or solution dosage form can have a unit weight of at least 10 mg. In some embodiments, a tablet, capsule, caplet, gel cap, powder, or solution dosage form can have a unit weight of less than 10 g. In some embodiments, a therapeutically effective amount of an LRRK2 inhibitor can be in a solution dosage form. In some embodiments, a solution dosage form can have a unit volume of at least 1 mL. In some embodiments, a solution dosage form can have a unit volume of less than 500 mL. In some embodiments, a therapeutically effective amount of an LRRK2 inhibitor can be in a powder dosage form. A LRRK2 inhibitor can comprise a small molecule, siRNA, or an antibody. A LRRK2 inhibitor can comprise PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, KN-62, KN-93, ML-7, ML-9, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, or K252a.

Disclosed herein are kits. In one aspect, the kit is a kit for treating or preventing multiple system atrophy in a subject in need thereof comprising an LRRK2 inhibitor and a written instruction for treating or preventing multiple system atrophy using an LRRK2 inhibitor. A LRRK2 inhibitor can comprise a small molecule, siRNA, or an antibody. A LRRK2 inhibitor can comprise PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, KN-62, KN-93, ML-7, ML-9, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, or K252a. In some embodiments, an LRRK2 inhibitor can be a therapeutically effective amount of LRRK2 inhibitor. In some embodiments, a therapeutically effective amount of an LRRK2 inhibitor can be at least 1 μg of an LRRK2 inhibitor per kg of a subject. In some embodiments, a therapeutically effective amount of an LRRK2 inhibitor can be less than 1000 mg of an LRRK2 inhibitor per kg of a subject. In some embodiments, a therapeutically effective amount of an LRRK2 inhibitor can be in a tablet, capsule, caplet, gel cap, powder, or solution dosage form. In some embodiments, a tablet, capsule, caplet, gel cap, powder, or solution dosage form can have a unit weight of at least 10 mg. In some embodiments, a tablet, capsule, caplet, gel cap, powder, or solution dosage form can have a unit weight of less than 10 g. In some embodiments, a therapeutically effective amount of an LRRK2 inhibitor can be in a solution dosage form. In some embodiments, a solution dosage form can have a unit volume of at least 1 mL. In some embodiments, a solution dosage form can have a unit volume of less than 500 mL. In some embodiments, a therapeutically effective amount of an LRRK2 inhibitor can be in a powder dosage form.

An aspect of the invention includes a method of screening a subject for a neurological disease, the method comprising: a) assaying a nucleic acid sample obtained from the subject by PCR, array Comparative Genomic Hybridization, sequencing, SNP genotyping, or fluorescent in situ hybridization to detect sequence information for more than one genetic loci; b) comparing the sequence information to a panel of nucleic acid biomarkers, wherein the panel can comprise at least one nucleic acid biomarker for each of the more than one genetic loci; and wherein the panel can comprise at least 2 low frequency nucleic acid biomarkers, wherein the low frequency nucleic acid biomarkers occur at a frequency of 0.1% or less in a population of subjects without a diagnosis of the neurological disease; and c) screening the subject for the presence or absence of the neurological disease if one or more of the low frequency biomarkers in the panel are present in the sequence information. The panel can comprise at least 5, 10, 25, 50, 100, or 200 low frequency nucleic acid biomarkers. The presence or absence of a neurological disease in the subject can be determined with at least 50% confidence. In some embodiments, the low frequency biomarkers occur at a frequency of 0.01% or less, 0.001% or less, or 0.0001% or less in a population of subjects without a diagnosis of the neurological disease. The panel of nucleic acid biomarkers can comprise at least two genes that encode at least a portion of LRRK2. The method can further comprise identifying a therapeutic agent useful for treating the neurological disease. In some embodiments, the neurological disease can be MSA.

An aspect of the invention includes a kit for screening a subject for a neurological disorder, the kit comprising at least one reagent for assaying a nucleic acid sample from the subject for information on a panel of nucleic acid biomarkers, wherein the panel comprises at least 2 low frequency biomarkers, and wherein the low frequency biomarkers occur at a frequency of 0.1% or less in a population of subjects without a diagnosis of the neurological disorder. In some embodiments, a presence or absence of the neurological disorder in the subject can be determined with a 50% confidence. The panel can comprise at least 5, 10, 25, 50, 100, or 200 low frequency nucleic acid biomarkers. In some embodiments, the low frequency biomarkers occur at a frequency of 0.01% or less, 0.001% or less, or 0.0001% or less in a population of subjects without a diagnosis of the neurological disorder. The at least one reagent can comprise at least two sets of suitable primers. The at least one reagent can comprise a reagent for the preparation of cDNA. The at least one reagent can comprise a reagent that can be used for detection and quantization of polynucleotides. The at least one reagent can comprise at least one chromophore.

An aspect of the invention includes a method of generating a panel of nucleic acid biomarkers comprising: a) assaying a nucleic acid sample from a first population of subjects by PCR, array Comparative Genomic Hybridization, sequencing, SNP genotyping, or fluorescent in situ hybridization for nucleic acid sequence information, wherein the subjects of the first population have a diagnosis of a neurological disorder; b) assaying a nucleic acid sample from a second population of subjects by PCR, array Comparative Genomic Hybridization, sequencing, SNP genotyping, or fluorescent in situ hybridization for nucleic acid sequence information, wherein the subjects of the second population are without a diagnosis of a neurological disorder; c) comparing the nucleic acid sequence information from step (a) to that of step (b); d) determining the frequency of one or more biomarkers from the comparing step; and e) generating the panel of a nucleic acid biomarkers, wherein the panel can comprise at least 2 low frequency biomarkers, and wherein the low frequency biomarkers occur at a frequency of 0.1% or less in a population of subjects without a diagnosis of a neurological disorder. The subjects in the second population of subjects without a diagnosis of a neurological disorder can comprise one or more subjects not suspected of having the neurological disorder. The subjects in the second population of subjects without a diagnosis of a neurological disorder can comprise one or more subjects without the neurological disorder. The subjects in the second population of subjects without a diagnosis of a neurological disorder can comprise one or more subjects who are asymptomatic for the neurological disorder. The subjects in the second population of subjects without a diagnosis of a neurological disorder can comprise one or more subjects who have decreased susceptibility to the neurological disorder. The subjects in the second population of subjects without a diagnosis of a neurological disorder can comprise one or more subjects who are unassociated with a treatment, therapeutic regimen, or any combination thereof. The panel can comprise at least 5, 10, 25, 50, 100, or 200 low frequency nucleic acid biomarkers. In some embodiments, the low frequency biomarkers occur at a frequency of 0.01% or less, 0.001% or less, or 0.0001% or less in the second population of subjects without a diagnosis of a neurological disorder. In some embodiments, the nucleic acid biomarker can be a sequence encoding a portion of LRRK2. In some embodiments the neurological disorder can be MSA.

In an aspect, disclosed herein are methods for identifying compounds for treating or preventing multiple system atrophy. The method can comprise providing a cell that expresses a gene that causes an LRRK2 mutation selected from Table 1. The method can comprise contacting the cell with a compound and assessing an expression level of the gene relative to the expression level of the gene in an absence of the compound, wherein if the compound reduces the expression level of the gene, the compound can be identified as useful for treating or preventing multiple system atrophy. The assessing can comprise measuring RNA levels transcribed from a gene. Assessing can comprise measuring an amount of an LRRK2 mutant protein encoded by a gene. A gene can be recombinantly expressed by a cell. The cell can be a mammalian cell. The mammalian cell can be a human cell or a rodent cell. The cell can be a dopaminergic cell or a cell isolated from the gray matter. The cell can be a glia cell. The glia cell can comprise an oligodendrocyte or an astrocyte. The cells can be a neuronal cell. The cell can be from or isolated from substantia nigra, brainstem, amygdala, hippocampus, temporal cortex, cingulate cortex, superior frontal gyms, motor cortex, caudate/putamen, globus pallidus, basal nucleus of Meynert, hypothalamus, thalamus, subthalamic nucleus, red nucleus, oculomotor complex, midbrain tectum, locus ceruleus, pontine tegmentum, pontine nuclei, medullary tegmentum, dentate nucleus cerebellar white matter or inferior olivary nucleus. The cell can overexpress α-synuclein.

In an aspect, disclosed herein are methods for identifying compounds for treating or preventing multiple system atrophy. The method can comprise providing a cell comprising a reporter gene operably linked to a promoter of a gene that causes an LRRK2 mutation selected from Table 1. The method can comprise contacting the cell with a compound; and assessing an expression level of the reporter gene relative to the expression level of the reporter gene in an absence of the compound, wherein if the compound reduces the expression level of the reporter gene, the compound can be identified as useful for treating or preventing multiple system atrophy. The reporter gene can be selected from the group of glucuronidase (GUS), luciferase, chloramphenicol transacetylase (CAT), green fluorescent protein (GFP), alkaline phosphatase, and β-galactosidase. The cell can be a mammalian cell. The mammalian cell can be a human cell or a rodent cell. The cell can be a dopaminergic cell or a cell isolated from the gray matter. The cell can be a glia cell. The glia cell can comprise an oligodendrocyte or an astrocyte. The cell can overexpress α-synuclein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in their entireties.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 depicts a schematic diagram of LRRK2 domains and select pathogenic mutations. LRRK2 has multiple protein domains including ANK (ankyrin-like repeat), LRR (leucine rich repeat), ROC (Ras of complex protein) GTPase, COR (C terminal of ROC), kinase, and WD40. ANK, LRR, and WD40 are protein-protein interaction domains. The ROC and kinase domains have enzymatic activity. Multiple mutations in LRRK2 are found in neurological diseases such as MSA and Parkinson.

FIG. 2 depicts an exemplary method for assessing MSA in a subject.

FIG. 3A depicts a pedigree. Circles in the pedigree indicate female gender, squares indicate male gender, full black symbol indicates MSA, and half black symbols indicate PD, but only historical information available. FIG. 3B depicts an electropherogram from Sanger sequencing. Boxed sequence indicates mutation and change in amino acid sequence from isoleucine to valine.

FIG. 4A depicts MSA-related neuropathology via H&E stain at 20 μm, NCIs and dystrophic neurites are found in inferior olivary nucleus. FIG. 4B depicts MSA-related neuropathology via α-synuclein immunohistochemistry at 20 μm, NCIs and dystrophic neurites are found in inferior olivary nucleus. FIG. 4C depicts MSA-related neuropathology via H&E stain at 20 μm, pontine nucleus has numerous NCIs and NIIs as well as dystrophic neurites. FIG. 4D depicts MSA-related neuropathology via α-synuclein immunohistochemistry at 20 μm, pontine nucleus has numerous NCIs and NIIs as well as dystrophic neurites. FIG. 4E depicts MSA-related neuropathology via H&E stain at 50 μm, cerebellum shows many GCI and extensive demyelination. FIG. 4F depicts MSA-related neuropathology via α-synuclein immunohistochemistry at 50 μm, cerebellum shows many GCI and extensive demyelination.

### DETAILED DESCRIPTION

### Overview

Disease and disease risk can be conferred by subtle changes in an individual genome. Genes can differ between individuals due to genomic variability, the most frequent of which are due to single nucleotide polymorphisms (SNPs). Additional genetic polymorphisms in a human genome can be caused by duplication, insertion, deletion, translocation, and/or inversion of short and/or long stretches of DNA. Genetic variations may encode protein variants that can result in an increased susceptibility to a disease or result in disease onset, for example neurodegenerative diseases such as multiple system atrophy (MSA). Multiple system atrophy is a rare, rapidly progressing neurodegenerative disorder characterized by a combination of symptoms that affect both the autonomic nervous system and central nervous system. Despite advances towards an understanding of the etiology of MSA and other neurological disorders, a large fraction of the genetic contribution to these disorders remains undetermined. Ideally, it would be desirable to provide additional tools to screen, detect, identify, and treat individuals that have or are at risk of developing neurological diseases, such as MSA.

Diseases such as MSA may be associated with one or more genetic variations, where the presence of a genetic variation may increase the risk of developing MSA or is indicative of MSA. Genetic analysis can be used to determine the presence of such a genetic variation. In instances where a subject presents symptoms for closely related diseases, for example MSA and Parkinson's disease, a genetic analysis can be used to differentiate related diseases. For example, a genetic analysis can be performed to determine a presence or absence of a diseases associated genetic variation. This approach can therefore rule out or confirm a disease. Thus, allowing for a correct diagnosis and proper treatment.

Described herein are methods to assess the risk for developing multiple system atrophy (MSA) by determining whether certain LRRK2 mutations are present. In particular, the presence of a genetic variation associated with MSA can be indicative of MSA or an increased risk of MSA. Following the detection of an LRRK2 mutation, an LRRK2 inhibitor can be administered to a subject to treat MSA or symptoms attributed to MSA.

To assess multiple system atrophy (MSA), nucleic acids can be extracted from a sample and purified. The purified nucleic acids can be incorporated in an amplification reaction with primers specific to a genetic variant sequence. The presence or absence of a specific genetic variation can thereafter be determined. In some cases, a purified nucleic acid can be sequenced to determine the presence or absence of a genetic variation. The presence of a genetic variation associated with MSA can be indicative of MSA or an increased risk of MSA. A genetic variation that can be indicative of MSA or an increased risk of MSA can be an LRRK2 genetic mutation and in some cases, the LRRK2 genetic variation can cause an LRRK2 I1371V mutation. In other cases, the LRRK2 genetic variation that is indicative of MSA or an increased risk of MSA may not cause an LRRK2 mutation at position L153, N551, 1723, L953, R1398, K1423, R1514, P1542, G1624, K1637, M1646, S1647, G1819, N2081, E2108, G2385, 12020, M2397, L153, N551, 1723, L953, R1398, K1423, R1514, P1542, G1624, K1637, M1646, S1647, G1819, N2081, E2108, G2385, 12020, or M2397. Following the detection of an LRRK2 mutation, an LRRK2 inhibitor can be administered to a subject to treat MSA or symptoms attributed to MSA. A therapeutically effective amount of an LRRK2 inhibitor can be administered orally, intraperitoneally, buccally, intravenously, parenterally, rectally, intradermally, transdermally, pulmonary, intracranially, nasally, topically, or by inhalation spray.

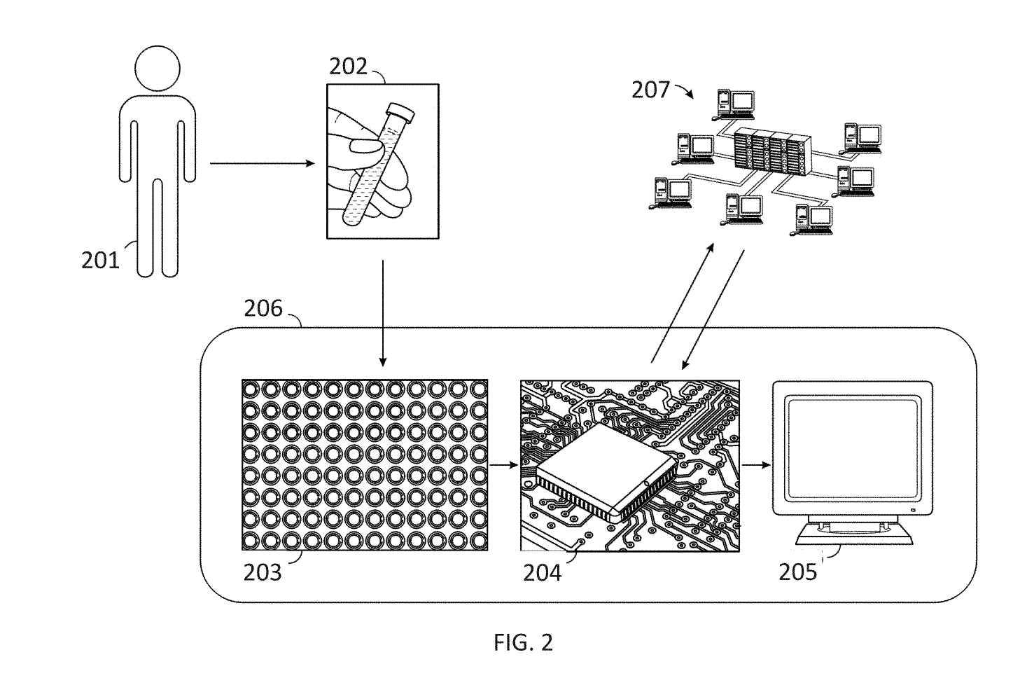

MSA can be assessed using a computer or a computer system. FIG. 2 shows an exemplary method for assessing MSA in a subject using a computer. A sample (FIG. 2, 202) can be obtained from a subject (FIG. 2, 201). A genetic variation or a protein mutation can be assessed (FIG. 2, 203) using a computer system (FIG. 2, 204). In some cases, the computer system can compare nucleic acid information of a sample to a reference. The reference can be stored in the computer system. Alternatively, the reference can be stored in other computers, databases, and/or servers, and accessible through a network (e.g., Internet) (FIG. 2, 207). The result of whether a subject has MSA can be transmitted to an output device, e.g., a monitor (FIG. 2, 205). The assay, the computer system, and the output device (FIGS. 2, 203, 204, and 205) can be integrated into a single device (FIG. 2, 206). In some cases, such a device can be a portable device, for example a smartphone. The device can be contemplated to be portable device for use in a hospital and/or a pre-hospital setting (e.g., in an ambulance or patient's home). Generally, a device can have a memory that stores executable instructions and a processor to execute the executable instructions to detect MSA.

### Definitions

The term “antibody” includes intact antibodies and binding fragments thereof. The term “antibody” also includes bispecific antibody, humanized antibody, monoclonal antibody, and polyclonal antibody.

The term “symptom” refers to a subjective evidence of a disease, such as altered gait, as perceived by the patient. A “sign” refers to objective evidence of a disease as observed by a physician.

“Cognitive function” refers to mental processes such as any, all of, but not limited to, attention, memory, producing and understanding language, solving problems, and taking an interest in one's surroundings and self-care “Enhanced cognitive function” or “improved cognitive function” refers to improvement relative to a baseline, for example, diagnosis or initiation of treatment. “Decline of cognitive function” refers to a decrease in function relative to such a base line.

“Pharmaceutically acceptable” refers to molecular entities and compositions that are physiologically tolerable and do not typically produce an allergic or similar untoward reaction, such as gastric upset, dizziness, and the like, when administered to a human.

A “packaging material” refers to a physical structure housing the components of a kit. The packaging material can maintain the components sterilely and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method described herein.

“Prevention” refers to prophylaxis, prevention of onset of symptoms, or prevention of progression of a disease or disorder. “Inhibition,” “treatment,” and “treating” are used interchangeably and refer to, for example, stasis of symptoms, prolongation of survival, partial or full amelioration of symptoms, and partial or full eradication of a condition, disease, or disorder.

Nucleotide,” “nucleoside,” “nucleotide residue,” and “nucleoside residue,” as used herein, can mean a deoxyribonucleotide or ribonucleotide residue, or other similar nucleoside analogue. A “nucleic acid,” or grammatical equivalents, refers to either a single nucleotide or at least two nucleotides covalently linked together.

A “polynucleotide” or grammatical equivalents refers to at least two nucleotides covalently linked together. A polynucleotide comprises a molecule containing two or more nucleotides. A polynucleotide comprises a specific sequence of four nucleotide bases: adenine (A); cytosine (C); guanine (G); and thymine (T) (uracil (U) for thymine (T) when the polynucleotide is RNA).

A “polypeptide” refers to a molecule comprising at least two amino acids. A polypeptide can comprise a single peptide. A polypeptide can comprise two or more peptides. Examples of polypeptides include, but are not limited to, amino acid chains, proteins, peptides, hormones, polypeptide saccharides, lipids, glycolipids, phospholipids, antibodies, enzymes, kinases, receptors, transcription factors, and ligands.

A “subject,” “individual,” “host,” or “patient” refers to a living organism such as mammals. Examples of subjects include, but are not limited to, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks) fish (e.g., sharks) or frogs (e.g., Xenopus), and non-mammalian invertebrates, as well as transgenic species thereof. In certain aspects, a subject refers to a single organism (e.g., human). A subject from whom a sample is obtained can either be afflicted with a disease and/or disorder and can be compared against a negative control subject which is not affected by the disease.

A “kit” refers to a delivery system for delivering materials or reagents for carrying out a method disclosed herein. Kits can include systems that allow for the storage, transport, or delivery of reaction reagents (e.g., probes, enzymes, etc. in the appropriate containers) and/or supporting materials (e.g., buffers, written instructions for performing the assessment etc.) from one location to another. For example, kits can include one or more enclosures (e.g., boxes) containing the relevant reaction reagents and/or supporting materials. Such contents can be delivered to the intended recipient together or separately. For example, a first container can contain an enzyme for use in an assay, while a second container can contain a plurality of primers.

“Treat” or “treatment” refers to a therapeutic treatment wherein the object is to eliminate or lessen symptoms.

Human gene symbols generally are italicized, with all letters in uppercase for example, LRRK2. Human protein designations are the same as the gene symbol, but are generally not italicized (LRRK2).

### Genetic Variations and Neurological Disorders

Genomic sequences within populations exhibit variability between individuals at many locations in the genome. For example, the human genome exhibits sequence variations that can occur on average every 500 base pairs. Such genetic variations in nucleic acid sequences are commonly referred to as polymorphisms or polymorphic sites. As used herein, a polymorphism, e.g., genetic variation, includes a variation in the sequence of a gene in the genome amongst a population, such as allelic variations and other variations that arise or are observed. Thus, a polymorphism can refer to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, DNA synthesis; expressed proteins, other gene products or products of biochemical pathways or in post-translational modifications and any other differences manifested amongst members of a population. A single nucleotide polymorphism (SNP) includes to a polymorphism that arises as the result of a single base change, such as an insertion, deletion or change in a base. A polymorphic marker or site is the locus at which divergence occurs. Such site can be as small as one base pair (an SNP). Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different Mendelian alleles for a gene. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

As used herein, “genetic variation” includes point mutations, polymorphisms, translocations, insertions, deletions, amplifications, inversions, interstitial deletions, copy number variations (CNVs), loss of heterozygosity, or any combination thereof. As genetic variation includes any deletion, insertion or base substitution of the genomic DNA of one or more individuals in a first portion of a total population which thereby results in a difference at the site of the deletion, insertion or base substitution relative to one or more individuals in a second portion of the total population. Thus, the term “genetic variation” encompasses “wild type” or the most frequently occurring variation, and also includes “mutant,” or the less frequently occurring variation. In some cases, a genetic variation can be a variation as compared to a wild type sequence.

Polymorphisms (e.g., polymorphic markers, genetic variations, or genetic variants) can comprise any nucleotide position at which two or more sequences are possible in a subject population. In some cases, each version of a nucleotide sequence with respect to the polymorphism can represent a specific allele, of the polymorphism. Genomic DNA from a subject can contain two alleles for any given polymorphic marker, representative of each copy of the marker on each chromosome. In some cases, an allele can be a nucleotide sequence of a given location on a chromosome. Polymorphisms can comprise any number of specific alleles. In some cases of the disclosure, a polymorphism can be characterized by the presence of two or more alleles in a population. A polymorphism can be characterized by the presence of three or more alleles. An allele can be associated with one or more diseases or disorders, for example, a neurological disorder risk allele can be an allele that is associated with increased or decreased risk of developing a neurological disorder. Genetic variations and alleles can be used to associate an inherited phenotype, for example, a neurological disorder, with a responsible genotype. In some cases, a neurological disorder risk allele can be a variant allele that is statistically associated with a screening of one or more neurological disorders. In some cases, genetic variations can be of any measurable frequency in the population, for example, a frequency higher than 10%, a frequency between 5-10%, a frequency between 1-5%, or frequency below 1%. As used herein, variant alleles can be alleles that differ from a reference allele. As used herein, a variant can be a segment of DNA that differs from a reference DNA, such as a genetic variation. Genetic variations can be used to track the inheritance of a gene that has not yet been identified, but whose approximate location is known.

As used herein, a “haplotype” can be information regarding the presence or absence of one or more genetic markers in a given chromosomal region in a subject. A haplotype can be a segment of DNA characterized by one or more alleles arranged along the segment, for example, a haplotype can comprise one member of the pair of alleles for each genetic variation or locus. In some cases, the haplotype can comprise two or more alleles, three or more alleles, four or more alleles, five or more alleles, or any combination thereof, wherein, each allele can comprise one or more genetic variations along the segment.

A genetic variation can be a functional aberration that can alter gene function, gene expression, polypeptide expression, polypeptide function, or any combination thereof. A genetic variation can be a loss-of-function mutation, gain-of-function mutation, dominant negative mutation, or reversion. A genetic variation can be part of a gene's coding region or regulatory region. Regulatory regions can control gene expression and thus polypeptide expression. In some cases, a regulatory region can be a segment of DNA wherein regulatory polypeptides, for example, transcription factors, can bind. A regulatory region can be positioned near the gene being regulated, for example, positions upstream of the gene being regulated. A regulatory region (e.g., enhancer element) can be several thousands of base pairs upstream or downstream of a gene.

Variants can include changes that affect a polypeptide, such as a change in expression level, sequence, function, localization, binding partners, or any combination thereof. In some cases, a genetic variation can be a frameshift mutation, nonsense mutation, missense mutation, neutral mutation, or silent mutation. For example, sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. A genetic variation associated with a neurological disorder can be a synonymous change in one or more nucleotides, for example, a change that does not result in a change in the amino acid sequence. Such a polymorphism can, for example, alter splice sites, affect the stability or transport of mRNA, or otherwise affect the transcription or translation of an encoded polypeptide. A synonymous mutation can result in the polypeptide product having an altered structure due to rare codon usage that impacts polypeptide folding during translation, which in some cases may alter its function and/or drug binding properties if it is a drug target. The changes that can alter DNA and increase the possibility that structural changes, such as amplifications or deletions, occur at the somatic level. A polypeptide encoded by a reference nucleotide sequence can be a reference polypeptide with a particular reference amino acid sequence, and polypeptides encoded by variant nucleotide sequences can be variant polypeptides with variant amino acid sequences.

One or more variant polypeptides can be associated with one or more diseases or disorders, such as MSA. Variant polypeptides and changes in expression, localization, and interaction partners thereof, can be used to associate an inherited phenotype, for example, a neurological disorder, with a responsible genotype. A neurological disorder associated variant polypeptide can be statistically associated with a diagnosis, prognosis, or theranosis of one or more neurological disorders. A LRRK2, PARK2, PARK7, PINK1, GBA, RAB7L1, EIF4G1, ATP13A2, SNCA, PARKIN, DJ1, PLA2G6, FBX07, GIGYF2, HTRA2, MAPT, BST1, GAK, APP, PS1, PS2, SOD1, P102L, 6-OPRI, E200K, PLA2G6, PANK2, FTL or, UCHL1 genetic variation can be associated with a neurological disorder or disease such as MSA. A LRRK2, PARK2, PARK7, PINK1, GBA, RAB7L1, EIF4G1, ATP13A2, SNCA, Parkin, DJ1, PLA2G6, FBX07, GIGYF2, HTRA2, MAPT, BST1, GAK, APP, P51, PS2, SOD1, P102L, 6-OPRI, E200K, PLA2G6, PANK2, FTL, or UCHL1 protein mutation can be associated with a neurological disorder or disease such as MSA. Neurological disorder and neurological disease are used interchangeably. In some cases, a protein or gene mutation can be a prion protein or gene mutation. “Neurological disorder,” “neurological diseases,” and “neurodegenerative disease” are use interchangeable.

The most common sequence variants comprise base variations at a single base position in the genome, and such sequence variants, or polymorphisms, are commonly called single nucleotide polymorphisms (SNPs) or single nucleotide variants (SNVs). In some cases, an SNP represents a genetic variant present at greater than or equal to 1% occurrence in a population. In some cases, an SNP can represent a genetic variant present at any frequency level in a population. A SNP can be a nucleotide sequence variation occurring when a single nucleotide at a location in the genome differs between members of a species or between paired chromosomes in a subject. SNPs can include variants of a single nucleotide, for example, at a given nucleotide position, some subjects can have a ‘G’, while others can have a ‘C’. SNPs can occur in a single mutational event, and therefore there can be two possible alleles possible at each SNP site; the original allele and the mutated allele. SNP polymorphisms can have two alleles, for example, a subject can be homozygous for one allele of the polymorphism wherein both chromosomal copies of the individual have the same nucleotide at the SNP location, or a subject can be heterozygous wherein the two sister chromosomes of the subject contain different nucleotides. The SNP nomenclature as reported herein is the official Reference SNP (rs) ID identification tag as assigned to each unique SNP by the National Center for Biotechnological Information (NCBI). In some cases SNPs can affect susceptibility to neurological disorders.

Another genetic variation of the disclosure can be copy number variations (CNVs). As used herein, “CNVs” include alterations of the DNA of a genome that results an abnormal number of copies of one or more sections of DNA. Other types of sequence variants can be found in the human genome and can be associated with a disease or disorder, including but not limited to, microsatellites. A polymorphic microsatellite can comprise multiple small repeats of bases, for example, CA repeats, at a particular site wherein the number of repeat lengths varies in a population. In some cases, microsatellites, for example, variable number of tandem repeats (VNTRs), can be short segments of DNA that have one or more repeated sequences, for example, about 2 to 5 nucleotides long, that can occur in non-coding DNA. In some cases, changes in microsatellites can occur during genetic recombination of sexual reproduction, increasing or decreasing the number of repeats found at an allele, or changing allele length.

### Neurological Disorders

“Neurological disorders,” as used herein, include Acquired Epileptiform Aphasia, Acute Disseminated Encephalomyelitis, Adrenoleukodystrophy, Agenesis of the corpus callosum, Agnosia, Aicardi syndrome, Alexander disease, Alpers' disease, Alternating hemiplegia, Alzheimer's disease, Amyotrophic lateral sclerosis (see Motor Neuron Disease), Anencephaly, Angelman syndrome, Angiomatosis, Anoxia, Aphasia, Apraxia, Arachnoid cysts, Arachnoiditis, Arnold-Chiari malformation, Arteriovenous malformation, Asperger's syndrome, Ataxia Telangiectasia, Attention Deficit Hyperactivity Disorder, Autism, Auditory processing disorder, Autonomic Dysfunction, Back Pain, Batten disease, Behcet's disease, Bell's palsy, Benign Essential Blepharospasm, Benign Focal Amyotrophy, Benign Intracranial Hypertension, Bilateral frontoparietal polymicrogyria, Binswanger's disease, Blepharospasm, Bloch-Sulzberger syndrome, Brachial plexus injury, Brain abscess, Brain damage, Brain injury, Brain tumor, Brown-Sequard syndrome, Canavan disease, Carpal tunnel syndrome (CTS), Causalgia, Central pain syndrome, Central pontine myelinolysis, Centronuclear myopathy, Cephalic disorder, Cerebral aneurysm, Cerebral arteriosclerosis, Cerebral atrophy, Cerebral gigantism, Cerebral palsy, Charcot-Marie-Tooth disease, Chiari malformation, Chorea, Chronic inflammatory demyelinating polyneuropathy (CIDP), Chronic pain, Chronic regional pain syndrome, Coffin Lowry syndrome, Coma, including Persistent Vegetative State, Congenital facial diplegia, Corticobasal degeneration, Cranial arteritis, Craniosynostosis, Creutzfeldt-Jakob disease, Cumulative trauma disorders, Cushing's syndrome, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Neurological Dyspraxia, Diabetic neuropathy, Diffuse sclerosis, Dysautonomia, Dyscalculia, Dysgraphia, Dyslexia, Dystonia, Early infantile epileptic encephalopathy, Empty sella syndrome, Encephalitis, Encephalocele, Encephalotrigeminal angiomatosis, Encopresis, Epilepsy, Erb's palsy, Erythromelalgia, Essential tremor, Fabry's disease, Fahr's syndrome, Fainting, Familial spastic paralysis, Febrile seizures, Fisher syndrome, Friedreich's ataxia, FART Syndrome, Gaucher's disease, Gerstmann's syndrome, Giant cell arteritis, Giant cell inclusion disease, Globoid cell Leukodystrophy, Gray matter heterotopia, Guillain-Barre syndrome, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile phytanic acid storage disease, Infantile Refsum disease, Infantile spasms, Inflammatory myopathy, Intracranial cyst, Intracranial hypertension, Joubert syndrome, Kearns-Sayre syndrome, Kennedy disease, Kinsbourne syndrome, Klippel Feil syndrome, Krabbe disease, Kugelberg-Welander disease, Kuru, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Lewy body dementia, Lissencephaly, Locked-In syndrome, Lou Gehrig's disease, Lumbar disc disease, Lyme disease—Neurological Sequelae, Machado-Joseph disease (Spinocerebellar ataxia type 3), Macrencephaly, Maple Syrup Urine Disease, Megalencephaly, Melkersson-Rosenthal syndrome, Menieres disease, Meningitis, Menkes disease, Metachromatic leukodystrophy, Microcephaly, Migraine, Miller Fisher syndrome, Mini-Strokes, Mitochondrial Myopathies, Mobius syndrome, Monomelic amyotrophy, Motor Neuron Disease, Motor skills disorder, Moyamoya disease, Mucopolysaccharidoses, Multi-Infarct Dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Niemann-Pick disease, Non 24-hour sleep-wake syndrome, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Overuse syndrome, Palinopsia, Paresthesia, Parkinson's disease, Paramyotonia Congenita, Paraneoplastic diseases, Paroxysmal attacks, Parry-Romberg syndrome (also known as Rombergs Syndrome), Pelizaeus-Merzbacher disease, Periodic Paralyses, Peripheral neuropathy, Persistent Vegetative State, Pervasive neurological disorders, Photic sneeze reflex, Phytanic Acid Storage disease, Pick's disease, Pinched Nerve, Pituitary Tumors, PMG, Polio, Polymicrogyria, Polymyositis, Porencephaly, Post-Polio syndrome, Postherpetic Neuralgia (PHN), Postinfectious Encephalomyelitis, Postural Hypotension, Prader-Willi syndrome, Primary Lateral Sclerosis, Prion diseases, Progressive Hemifacial Atrophy also known as Rombergs Syndrome, Progressive multifocal leukoencephalopathy, Progressive Sclerosing Poliodystrophy, Progressive Supranuclear Palsy, Pseudotumor cerebri, Ramsay-Hunt syndrome (Type I and Type II), Rasmussen's encephalitis, Reflex sympathetic dystrophy syndrome, Refsum disease, Repetitive motion disorders, Repetitive stress injury, Restless legs syndrome, Retrovirus-associated myelopathy, Rett syndrome, Reye's syndrome, Rombergs Syndrome, Rabies, Saint Vitus dance, Sandhoff disease, Schytsophrenia, Schilder's disease, Schizencephaly, Sensory Integration Dysfunction, Septo-optic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal stenosis, Steele-Richardson-Olszewski syndrome, see Progressive Supranuclear Palsy, Spinocerebellar ataxia, Stiff-person syndrome, Stroke, Sturge-Weber syndrome, Subacute sclerosing panencephalitis, Subcortical arteriosclerotic encephalopathy, Superficial siderosis, Sydenham's chorea, Syncope, Synesthesia, Syringomyelia, Tardive dyskinesia, Tay-Sachs disease, Temporal arteritis, Tethered spinal cord syndrome, Thomsen disease, Thoracic outlet syndrome, Tic Douloureux, Todd's paralysis, Tourette syndrome, Transient ischemic attack, Transmissible spongiform encephalopathies, Transverse myelitis, Traumatic brain injury, Tremor, Trigeminal neuralgia, Tropical spastic paraparesis, Trypanosomiasis, Tuberous sclerosis, Vasculitis including temporal arteritis, Von Hippel-Lindau disease (VHL), Viliuisk Encephalomyelitis (VE), Wallenberg's syndrome, Werdnig-Hoffman disease, West syndrome, Whiplash, Williams syndrome, Wilson's disease, X-Linked Spinal and Bulbar Muscular Atrophy, and Zellweger syndrome. Neurological conditions can comprise movement disorders, for example multiple system atrophy (MSA).

### Multiple System Atrophy

Multiple system atrophy (MSA) is a rare condition that causes symptoms similar to Parkinson disease. However, subjects with MSA have more widespread damage to the part of the nervous system that controls important functions such as heart rate, blood pressure, and sweating. MSA damages the nervous system. Symptoms can include any one or more of the following: face changes, such as a masklike appearance to the face and staring; difficulty chewing or swallowing (occasionally), not able to close the mouth; disrupted sleep patterns (especially during rapid eye movement (REM) sleep late at night); dizziness or fainting when standing up or after standing still; frequent falls; erection problems; loss of control over bowels or bladder; problems with activity that requires small movements (loss of fine motor skills), such as writing that is small and hard to read; loss of sweating in any part of the body; mild decline in mental function; movement difficulties, such as loss of balance, shuffling when walking; muscle aches and pains (myalgia), and stiffness; nausea and problems with digestion; posture problems, such as unstable, stooped, or slumped over; slow movements; tremors; vision changes, decreased or blurred vision; voice and speech changes; confusion; dementia; depression; sleep-related breathing difficulties, especially sleep apnea or a blockage in the air passage that leads to a harsh vibrating sound. Additional symptoms of MSA may include: contractures (chronic shortening of muscles or tendons around joints, which prevents the joints from moving freely) in the hands or limbs; Pisa syndrome, an abnormal posture in which the body appears to be leaning to one side like the Leaning Tower of Pisa; antecollis, in which the neck bends forward and the head drops down; involuntary, uncontrollable sighing or gasping sleep disorders, including a tendency to act out dreams (called REM/(Rapid Eye Movement sleep behavior disorder).

MSA can be divided into two different types, depending on the most prominent symptoms at the time an individual is evaluated: (1) the parkinsonian type (MSA-P), with primary characteristics similar to Parkinson's disease (such as moving slowly, stiffness, and tremor) along with problems of balance, coordination, and autonomic nervous system dysfunction; (2) the cerebellar type (MSA-C), with primary symptoms featuring ataxia (problems with balance and coordination), difficulty swallowing, speech abnormalities or a quavering voice, and abnormal eye movements. In some embodiments, the methods disclosed herein can differentiate the types of MSA. MSA tends to progress more rapidly than Parkinson's disease, and most subjects with MSA will require an aid for walking, such as a cane or walker, within a few years after symptoms begin.

Prior to this invention, there existed no specific tests to detect MSA. A diagnosis of MSA can be based on: history of symptoms, physical examination, and ruling out other causes of symptoms. Testing to help confirm the diagnosis can include: use of imaging techniques such as MRI, PET scan, or ultrasound to evaluate the brain, an evaluation of plasma norepinephrine levels and/or a urine examination for norepinephrine breakdown products (urine catecholamines). In some embodiments, the methods disclosed herein can be used to monitor a neurological disorder such as MSA. To monitor a neurological disorder, a method as disclosed herein can be repeated to assess a subject. Detection of a genetic variation or LRRK2 mutation disclosed herein can be used in combination with one or more imaging techniques such as MRI, PET scan, or ultrasound to evaluate the brain, an evaluation of plasma norepinephrine levels and/or a urine examination for norepinephrine breakdown products to detect a neurological disorder or a risk of developing a neurological disorder or susceptibility to a neurological disorder. In some embodiments, a detection of an LRRK2 p.I137V (c.4111A>G) mutation and abnormal MRI scan can indicate MSA.

F-FDG PET scan can be highly sensitive for detecting atypical Parkinsonian syndromes such as MSA. Diffusion weighted MRI can show the movement of water through the brain; higher or lower than normal levels of water can indicate dead or dying brain tissue. In some embodiments, normal results on diffusion MRI point toward a diagnosis of Parkinson's, while abnormal results can indicate possible MSA, progressive supranuclear palsy (PSP) or corticobasilar degeneration (CBD). In some embodiments, on diffusion-weighted MRI, patients with MSA and/or PSP can show a specific pattern of atrophy the putamen. In some embodiments, neuromelanin MRI can detect nerve cells in the substantia nigra. Degeneration of this brain structure occurs in both Parkinson's disease and MSA. In some embodiments, neuromelanin MRI can distinguish MSA from Parkinson's disease. In some embodiments, a sweat test and/or a sleep evaluation can be used to confirm or diagnose MSA. Quantitative Sudomotor Autonomic Reflex Test (QSART) or polysomnography can be used to confirm MSA. In some embodiments, the methods disclosed herein can be used to detect or confirm a neurological disorder, for example MSA.

The cause of MSA is unknown. The vast majority of cases are sporadic. A distinguishing feature of MSA is the accumulation of the protein alpha-synuclein in glia, (oligodendroglia). Alpha-synuclein also accumulates in Parkinson's disease, but in nerve cells. Because both Parkinson's disease and MSA have a buildup of alpha-synuclein in cells, MSA and Parkinson's disease are sometimes referred to as synucleinopathies. A possible risk factor for the disease is variations in the synuclein gene SNCA, which provides instructions for the production of alpha-synuclein.

Prior to this invention, there existed no treatment for MSA, however treatments can be deployed to treat MSA symptoms. Anticholinergic medicines, such as levodopa or carbidopa, can be used to reduce early or mild tremors, however, the benefit may not continue as the disease progresses. The fainting and lightheadedness from orthostatic hypotension symptoms of MSA can be treated with interventions such as wearing compression stockings, adding extra salt and/or water to the diet, and avoiding heavy meals. Fludrocortisone, droxidopa, and midodrine can also be used to treat these symptoms. Dihydroxyphenylserine can help replace neurotransmitters which are decreased in the autonomic nervous system in MSA.

Bladder control symptoms of MSA can be treated according to the nature of the problem. Anticholinergic drugs, such as oxybutynin or tolteridine, can be used to reduce the sudden urge to urinate. Fixed abnormal muscle postures (dystonia) symptoms of MSA can be controlled with injections of botulinum toxin. Sleep symptoms of MSA such as REM sleep behavior disorder can be treated with medicines including clonazepam, melatonin, or antidepressants. Speech therapy can be helpful in identifying strategies to address swallowing difficulties in MSA. In some embodiments, physical therapy can help maintain mobility, reduce contractures (chronic shortening of muscles or tendons around joints, which prevents the joints from moving freely), and decrease muscle spasms and abnormal posture. Individuals with MSA may eventually need assistive devices such as walkers and wheelchairs. Occupational therapists can help with home safety and learning new ways to address activities of daily living such as dressing and eating. In some embodiments, the treatments disclosed herein can be use alone or in combination.

### LRRK2 and Multiple System Atrophy

LRRK2 is a large protein (280 KDa). Sequence homology analysis and functional characterization reveal LRRK2 has the highest similarity to mixed-lineage kinases (MLK) that typically have both serine/threonine and tyrosine kinase activities. MLKs are part of the mitogen-activated protein kinase (MAPK) family and act as MAPK kinase kinases (MAPKKKs) to initiate and transduce a wide range of cellular responses. Consistent with the possibility that LRRK2 may function as an MLK/MAPKKK are the extent and the number of physiologic process that may be regulated by LRRK2. These include a role in neurite outgrowth and guidance, protein translation through regulation of microRNA processing and vesicle storage and mobilization within the recycling pool.

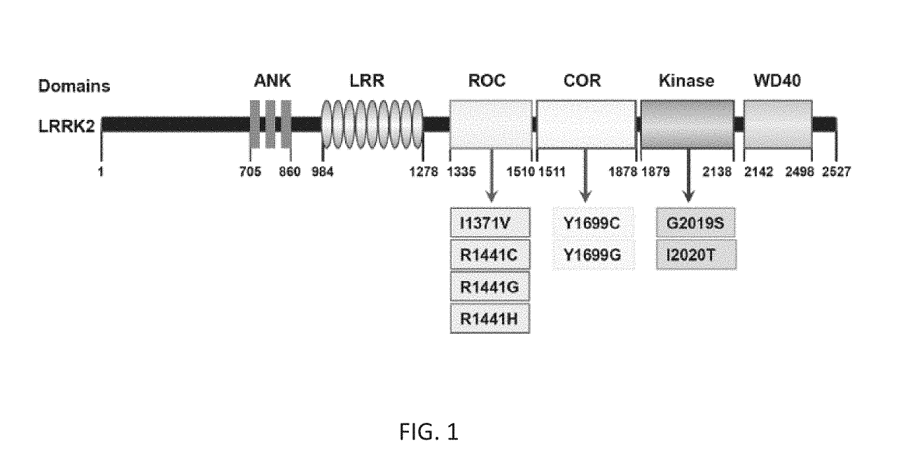

LRRK2 has multiple protein domains (FIG. 1), including protein-protein binding domains, such as the LRR domain and the WD40 domain. LRRK2 also has two distinct but functionally linked enzymatic domains, a Ras of complex (Roc) GTPase domain and a protein kinase domain that are linked by a carboxy-terminal of Roc (COR) sequence. Notably, multiple pathogenic mutations, for example I1371V, R1441C, R1441G, R1441H, Y1699C, Y1699G, G2019S, and 12020T, are located within the GTPase and the kinase domains or within the COR domain (FIG. 1, Table 1). Many LRRK2 mutants have abnormally high kinase activity when compared to wild-type LRRK2. In some instance, LRRK2 mutants can be associated with neuronal toxicity and degeneration of dopaminergic neurons. In some embodiments, neuronal toxicity may not be caused by LRRK2 variants that harbor a kinase-inactivating point mutation. In some embodiments, the LRRK2 gene provides instructions for the LRRK2 protein.

In some embodiments, an LRRK2 genetic variation can be associated with MSA. A non-limiting list of LRRK2 genetic variations and LRRK2 mutations can be found in Table 1. In some embodiments, an LRRK2 genetic variation that is associated with MSA may not cause an LRRK2 mutation at positon L153, N551, 1723, L953, R1398, K1423, R1514, P1542, G1624, K1637, M1646, S1647, G1819, N2081, E2108, G2385, 12020, or M2397. In some embodiments, an LRRK2 genetic variation that is associated with MSA LRRK2 may not cause an LRRK2 mutation at positon 1723, R1441, R1628, M1869, G2019, T1699, 12012 or T2356. In some embodiments, an LRRK2 genetic variation associated with MSA LRRK2 can cause an LRRK2 mutation at positon I1371. In some embodiments, an LRRK2 genetic variation associated with MSA and causes an LRRK2 mutation at positon at position I1371 can be an I1371V mutation. A LRRK2 mutation at the disclosed positions can result in the accumulation of Lewy bodies, substantia nigra neuronal loss, neurofibrillary tangles, and/or ubiquitin staining.

A neurodegenerative disease, for example MSA comprises a leucine-rich repeat kinase 2 (LRRK2) protein mutation, e.g., a human LRRK2 protein. Human LRRK2 has the following standard amino acid sequence (GenBank and NCBI Reference Sequence Accession Number NP_940980.3):

MASGSCQGCEEDEETLKKLIVRLNNVQEGKQIETLVQILEDLLVFTYSEH
ASKLFQGKNIHVPLLIVLDSYMRVASVQQVGWSLLCKLIEVCPGTMQSLM
GPQDVGNDWEVLGVHQLILKMLTVHNASVNLSVIGLKTLDLLLTSGKITL
LILDEESDIFMLIFDAMHSFPANDEVQKLGCKALHVLFERVSEEQLTEFV
ENKDYMILLSALTNFKDEEEIVLHVLHCLHSLAIPCNNVEVLMSGNVRCY
NIVVEAMKAFPMSERIQEVSCCLLHRLTLGNFFNILVLNEVHEFVVKAVQ
QYPENAALQISALSCLALLTETIFLNQDLEEKNENQENDDEGEEDKLFWL
EACYKALTWHRKNKHVQEAACWALNNLLMYQNSLHEKIGDEDGHFPAHRE
VMLSMLMHSSSKEVFQASANALSTLLEQNVNFRKILLSKGIHLNVLELMQ
KHIHSPEVAESGCKMLNHLFEGSNTSLDIMAAVVPKILTVMKRHETSLPV
QLEALRAILHFIVPGMPEESREDTEFHHKLNMVKKQCFKNDIHKLVLAAL
NRFIGNPGIQKCGLKVISSIVHFPDALEMLSLEGAMDSVLHTLQMYPDDQ
EIQCLGLSLIGYLITKKNVFIGTGHLLAKILVSSLYRFKDVAEIQTKGFQ
TILAILKLSASFSKLLVHHSFDLVIFHQMSSNIMEQKDQQFLNLCCKCFA
KVAMDDYLKNVMLERACDQNNSIMVECLLLLGADANQAKEGSSLICQVCE
KESSPKLVELLLNSGSREQDVRKALTISIGKGDSQIISLLLRRLALDVAN
NSICLGGFCIGKVEPSWLGPLFPDKTSNLRKQTNIASTLARMVIRYQMKS
AVEEGTASGSDGNFSEDVLSKFDEWTFIPDSSMDSVFAQSDDLDSEGSEG
SFLVKKKSNSISVGEFYRDAVLQRCSPNLQRHSNSLGPIFDHEDLLKRKR
KILSSDDSLRSSKLQSHMRHSDSISSLASEYITSLDLSANELRDIDALSQ
KCCISVHLEHLEKLELHQNALTSFPQQLCETLKSLTHLDLHSNKFTSFPS
YLLKMSCIANLDVSRNDIGPSVVLDPTVKCPTLKQFNLSYNQLSFVPENL
TDVVEKLEQLILEGNKISGICSPLRLKELKILNLSKNHISSLSENFLEAC
PKVESFSARMNFLAAMPFLPPSMTILKLSQNKFSCIPEAILNLPHLRSLD
MSSNDIQYLPGPAHWKSLNLRELLFSHNQISILDLSEKAYLWSRVEKLHL
SHNKLKEIPPEIGCLENLTSLDVSYNLELRSFPNEMGKLSKIWDLPLDEL
HLNFDFKHIGCKAKDIIRFLQQRLKKAVPYNRMKLMIVGNTGSGKTTLLQ
QLMKTKKSDLGMQSATVGIDVKDWPIQIRDKRKRDLVLNVWDFAGREEFY
STHPHFMTQRALYLAVYDLSKGQAEVDAMKPWLFNIKARASSSPVILVGT
HLDVSDEKQRKACMSKITKELLNKRGFPAIRDYHFVNATEESDALAKLRK
TIINESLNFKIRDQLVVGQLIPDCYVELEKIILSERKNVPIEFPVIDRKR
LLQLVRENQLQLDENELPHAVHFLNESGVLLHFQDPALQLSDLYFVEPKW
LCKIMAQILTVKVEGCPKHPKGIISRRDVEKFLSKKRKFPKNYMSQYFKL
LEKFQIALPIGEEYLLVPSSLSDHRPVIELPHCENSEIIIRLYEMPYFPM
GFWSRLINRLLEISPYMLSGRERALRPNRMYWRQGIYLNWSPEAYCLVGS
EVLDNHPESFLKITVPSCRKGCILLGQVVDHIDSLMEEWFPGLLEIDICG
EGETLLKKWAKFNDGEEHQKILLDDLMKKAEEGDLLVNPDQPRLTIPISQ
IAPDLILADLPRNIMLNNDELEFEQAPEFLLGDGSFGSVYRAAYEGEEVA
VKIFNKHTSLRLLRQELVVLCHLHHPSLISLLAAGIRPRMLVMELASKGS
LDRLLQQDKASLTRTLQHRIALHVADGLRYLHSAMIIYRDLKPHNVLLFT
LYPNAAIIAKIADYGIAQYCCRMGIKTSEGTPGFRAPEVRNVIYNQQADV
YSFGLLLYDILTTGGRIVEGLKFPNEFDELEIQGKLPDPVKEYGCAPWPM
VEKLIKQCLKENPQERPTSAQVFDILNSAELVCLTRRILLPKNVIVECMV
ATHHNSRNASIWLGCGHTDRGQLSFLDLNTEGYTSEEVADSRILCLALVH
LPVEKESWIVSGTQSGTLLVINTEDGKKRHTLEKMTDSVTCLYCNSFSKQ
SKQKNFLLVGTADGKLAIFEDKTVKLKGAAPLKILNIGNVSTPLMCLSES
TNSTERNVMWGGCGTKIFSFSNDFTIQKLIETRTSQLFSYAAFSDSNIIT
VVVDTALYIAKQNSPVVEVWDKKTEKLCGLIDCVHFLREVMVKENKESKH
KMSYSGRVKTLCLQKNTALWIGTGGGHILLLDLSTRRLIRVIYNFCNSVR
VMMTAQLGSLKNVMLVLGYNRKNTEGTQKQKEIQSCLTVWDINLPHEVQN
LEKHIEVRKELAEKMRRTSVE.

Table 1. Discloses a non-limiting list of LRRK2 variants, for example, rs34637584 is an SNP indicating a position within the LRRK2 gene that encodes an LRRK2 variant protein. This SNP is commonly referred to as the G2019S variant (or, mutation) based on the potential change from glycine (encoded by rs34637584(G) allele) to serine (encoded by the rs34637584(A) allele) at position 2019 of the LRRK2 protein.

TABLE 1

| | | | | Amino Acid | |
|---|---|---|---|---|---|
| Position | Exon | rs# | cDNA | Variation | Domain |
| chr12: 38905228 | 1 | | 28G > A | E10K | |
| chr12: 38905349 | 1 | rs2256408 | 149G > A | R50H | |
| chr12: 38905627 | 2 | rs72546335 | 155C > T | S52F | |
| chr12: 38905696 | 2 | rs75054132 | 224G > A | A75A | |
| chr12: 38915703 | 4 | rs33995463 | 356T > C | L119P | |
| chr12: 38915711 | 4 | rs41286468 | 364T > C | L122L | |
| chr12: 38918058 | 5 | rs10878245 | 457T > C | L153L | |
| chr12: 38918147 | 5 | rs35517158 | 546A > G | K182K | |
| chr12: 38920612 | 6 | rs112794616 | 632C > T | A211V | |
| chr12: 38920663 | 6 | rs56108242 | 683G > C | C228S | |
| chr12: 38923625 | 7 | rs28365216 | 713A > T | N238I | |
| chr12: 38923737 | 7 | rs72546315 | 824C > T | H275H | |
| chr12: 38929923 | 8 | rs17490713 | 867T > C | N289N | |
| chr12: 38929949 | 8 | rs57355477 | 893T > C | A298A | |
| chr12: 38929992 | 8 | rs41286466 | 936G > T | A312A | |
| chr12: 38931342 | 9 | rs78501232 | 1000G > A | E334K | |
| chr12: 38931397 | 9 | rs36016791 | 1055delC | A352fsX357 | |
| chr12: 38931430 | 9 | rs72546336 | 1088A > G | N363S | |
| chr12: 38931438 | 9 | rs113065049 | 1096G > A | V366M | |
| chr12: 38933053 | 11 | rs34594498 | 1256C > T | A419V | |
| chr12: 38937411 | 12 | rs35847451 | 1383C > T | S461S | |
| chr12: 38939594 | 13 | rs75711334 | 1464A > T | L488L | |
| chr12: 38939673 | 13 | rs34090008 | 1543insG | P514fsX529 | |
| chr12: 38943875 | 14 | rs35328937 | 1561A > G | R521G | |
| chr12: 38943944 | 14 | rs79996249 | 1630 A > G | K544E | |
| chr12: 38943967 | 14 | rs7308720 | 1653C > G | N551K | |
| chr12: 38954669 | 15 | rs77424631 | 1647G > A | G558G | |
| chr12: 38958002 | 17 | rs78154388 | 1987T > C | S663P | |
| chr12: 38958037 | 17 | rs72546319 | 2022A > C | V674V | |
| chr12: 38958213 | 17 | rs35611877 | 2198insA | L708fsX718 | ANK |
| chr12: 38958223 | 18 | | 2134A > G | M712V | ANK |
| chr12: 38958236 | 18 | | 2147C > T | A716V | ANK |
| chr12: 38958256 | 18 | rs10878307 | 2167A > G | I723V | ANK |
| chr12: 38963966 | 19 | rs34410987 | 2264C > T | P755L | ANK |
| chr12: 38964080 | 19 | rs35173587 | 2378G > T | R793M | ANK |
| chr12: 38964130 | 19 | rs72546337 | 2428A > G | I810V | ANK |
| chr12: 38964183 | 19 | rs76890302 | 2481T > C | S827S | ANK |
| chr12: 38967530 | 20 | | 2611A > G | K871E | |
| chr12: 38973693 | 21 | rs58559150 | 2769G > C | Q923H | |
| chr12: 38973713 | 21 | | 2789A > G | Q930R | |
| chr12: 38974935 | 22 | rs17519916 | 2830G > T | D944Y | |
| chr12: 38974962 | 22 | rs7966550 | 2857T > C | L953L | |
| chr12: 38975535 | 23 | rs75148313 | 2918G > A | S973N | |
| chr12: 38975635 | 23 | rs113217062 | 3018A > G | I1006M | LRR |
| chr12: 38975638 | 23 | rs55783828 | 3021C > T | S1007S | LRR |
| chr12: 38978415 | 24 | rs111341148 | 3200G > A | R1067Q | LRR |
| chr12: 38978502 | 24 | rs76535406 | 3287C > G | S1096C | LRR |
| chr12: 38978548 | 24 | rs78365431 | 3333G > T | Q1111H | LRR |
| chr12: 38978557 | 24 | rs35808389 | 3342A > G | L1114L | LRR |
| chr12: 38979194 | 25 | rs34805604 | 3364A > G | I1122V | LRR |
| chr12: 38979281 | 25 | rs74985840 | 3451G > A | A1151T | LRR |
| chr12: 38979324 | 25 | | 3494T > C | L1165P | LRR |
| chr12: 38982935 | 26 | | 3574A > G | I1192V | LRR |
| chr12: 38984073 | 27 | rs72546324 | 3647A > G | H1216R | LRR |
| chr12: 38984109 | 27 | rs80179604 | 3683G > C | S1228T | LRR |
| chr12: 38984109 | 27 | rs60185966 | 3683G > T | S1228I | LRR |
| chr12: 38985860 | 28 | rs4640000 | 3784C > G | P1262A | LRR |
| chr12: 38988536 | 29 | rs77018758 | 3960G > C/T | R1320S | |
| chr12: 38988550 | 29 | rs72546338 | 3974G > A | R1325Q | |
| chr12: 38988687 | 29 | rs17466213 | 4111A > G | I1371V | Roc |
| chr12: 38988701 | 29 | rs28365226 | 4125C > A | D1375E | Roc |
| chr12: 38989178 | 30 | rs7133914 | 4193G > A | R1398H | Roc |
| chr12: 38989214 | 30 | rs72546327 | 4229C > T | T1410M | Roc |
| chr12: 38989243 | 30 | rs113589830 | 4258G > A | D1420N | Roc |
| chr12: 38989254 | 30 | rs11175964 | 4269G > A | K1423K | Roc |
| chr12: 38989275 | 30 | rs111435410 | 4290C > T | A1430A | Roc |
| chr12: 38989294 | 30 | rs74163686 | 4309A > C | N1437H | Roc |
| chr12: 38990503 | 31 | rs33939927 | 4321C > T | R1441C | Roc |
| chr12: 38990503 | 31 | rs33939927 | 4321C > G | R1441G | Roc |
| chr12: 38990504 | 31 | rs34995376 | 4322G > A | R1441H | Roc |
| chr12: 38990505 | 31 | rs112998035 | 4323C > T | R1441R | Roc |
| chr12: 38990506 | 31 | | 4324G > C | A1442P | Roc |
| chr12: 38990519 | 31 | rs74681492 | 4337C > T | P1446L | Roc |
| chr12: 38990530 | 31 | rs111501952 | 4348G > A | V1450I | Roc |
| chr12: 38990569 | 31 | rs35363614 | 4387insA | R1462fsX1468 | Roc |
| chr12: 38990584 | 31 | | 4402A > G | K1468E | Roc |
| chr12: 38990630 | 31 | rs113431708 | 4448G > A | R1483Q | Roc |
| chr12: 38994045 | 32 | rs35507033 | 4541G > A | R1514Q | COR |
| chr12: 38994128 | 32 | rs33958906 | 4624C > T | P1542S | COR |
| chr12: 38994170 | 32 | rs17491187 | 4666C > A | L1556I | COR |
| chr12: 38995335 | 33 | rs721710 | 4793T > A | V1598E | COR |
| chr12: 39000067 | 34 | | 4838T > C | V1613A | COR |
| chr12: 39000101 | 34 | rs1427263 | 4872C > A | G1624G | COR |
| chr12: 39000112 | 34 | rs33949390 | 4883G > C | R1628P | COR |
| chr12: 39000140 | 34 | rs11176013 | 4911A > G | K1637K | COR |
| chr12: 39000166 | 34 | rs35303786 | 4937T > C | M1646T | COR |
| chr12: 39000168 | 34 | rs11564148 | 4939T > A | S1647T | COR |
| chr12: 39,000,188 | 34 | rs111503579 | 4959A > G | L1653L | COR |
| chr12: 39001183 | 35 | rs35801418 | 5096A > G | Y1699C | COR |
| chr12: 39001350 | 35 | rs79909111 | 5163A > G | S1721S | COR |
| chr12: 39002106 | 36 | rs11564176 | 5173C > T | R1725STOP | COR |
| chr12: 39002116 | 36 | | 5183G > T | R1728L | COR |
| chr12: 39002116 | 36 | ss263192805 | 5183G > A | R1728H | COR |
| chr12: 39002455 | 37 | rs111910483 | 5385G > T | L1795F | COR |
| chr12: 39002527 | 37 | rs10878371 | 5457T > C | G1819G | COR |
| chr12: 39003324 | 38 | | 5605A > G | M1869V | COR |
| chr12: 39003325 | 38 | rs35602796 | 5606T > C | M1869T | COR |
| chr12: 39003329 | 38 | | 5610G > T | L1870F | COR |
| chr12: 39003339 | 38 | | 5620G > T | E1874STOP | COR |
| chr12: 39015100 | 39 | rs77428810 | 5822G > A | R1941H | MAPKKK |
| chr12: 39020430 | 41 | | 6016T > C | Y2006H | MAPKKK |
| chr12: 39020449 | 41 | rs34015634 | 6035T > C | I2012T | MAPKKK |
| chr12: 39020469 | 41 | rs34637584 | 6055G > A | G2019S | MAPKKK |
| chr12: 39020473 | 41 | rs35870237 | 6059T > C | I2020T | MAPKKK |
| chr12: 39020505 | 41 | rs78029637 | 6091A > T | T2031S | MAPKKK |
| chr12: 39026899 | 42 | rs111739194 | 6187delCTCTA | L2063STOP | MAPKKK |
| chr12: 39026953 | 42 | rs33995883 | 6241A > G | N2081D | MAPKKK |
| chr12: 39028521 | 43 | rs10878405 | 6324G > A | E2108E | MAPKKK |
| chr12: 39028553 | 43 | rs12423862 | 6356C > T | P2119L | MAPKKK |
| chr12: 39031648 | 44 | rs111691891 | 6422C > T | T2141M | |
| chr12: 39031736 | 44 | rs34869625 | 6510C > A | G2170G | WD40 |
| chr12: 39031792 | 44 | rs35658131 | 6566A > G | Y2189C | WD40 |
| chr12: 39036195 | 46 | rs12581902 | 6782A > T | N2261I | WD40 |
| chr12: 39043509 | 48 | rs113511708 | 7067C > T | T2356I | WD40 |
| chr12: 39043595 | 48 | rs34778348 | 7153G > A | G2385R | WD40 |
| chr12: 39043597 | 48 | rs33962975 | 7155A > G | G2385G | WD40 |
| chr12: 39043610 | 48 | rs79546190 | 7168G > A | V2390M | WD40 |
| chr12: 39044912 | 49 | rs78964014 | 7183G > A | E2395K | WD40 |
| chr12: 39044916 | 49 | rs111272009 | 7187insGT | T2356fsX2360 | WD40 |
| chr12: 39044919 | 49 | rs3761863 | 7190C > T | M2397T | WD40 |
| chr12: 39044953 | 49 | rs60545352 | 7224G > A | M2408I | WD40 |
| chr12: 39047081 | 50 | | 7397T > A | L2466H | WD40 |
| chr12: 39047119 | 50 | rs55633591 | 7435A > G | N2479D | WD40 |

### Subject

A “subject,” as used herein, can be an individual of any age or sex from whom a sample can be obtained. A subject can include for example, a male or female adult, child, newborn, or fetus. In some embodiments, a subject can be any target of therapeutic administration. In some embodiments, a subject can be a test subject or a reference subject. In some embodiments, a subject can be associated with a condition or disease or disorder, asymptomatic or symptomatic, have increased or decreased susceptibility to a disease or disorder, be associated or unassociated with a treatment or treatment regimen, or any combination thereof. As used in the present disclosure, a cohort can represent an ethnic group, a patient group, a particular age group, a group not associated with a particular disease or disorder, a group associated with a particular disease or disorder, a group of asymptomatic subjects, a group of symptomatic subjects, or a group or subgroup of subjects associated with a particular response to a treatment regimen or clinical trial. In some embodiments, a patient can be a subject afflicted with a disease or disorder. In some embodiments, a patient can be a subject not afflicted with a disease or disorder. In some embodiments, a subject can be a test subject, a patient, or a candidate for a therapeutic, wherein a sample from the subject, patient, or candidate is obtained for analysis by one or more methods of the present disclosure herein. In some embodiments, a sample can be obtained prenatally from a fetus or embryo or from the mother, for example, from fetal or embryonic cells in the maternal circulation.

The present disclosure also provides methods for assessing genetic variations in subjects who are members of a target population. Such a target population is in some embodiments a population or group of subjects at risk of developing the disease, based on, for example, other genetic factors, biomarkers, biophysical parameters, family history of a neurological disorder, previous screening or medical history, or any combination thereof.

Although MSA is known to affect older adults more frequently than children, subjects of all ages are contemplated in the present disclosure. In some embodiments subjects can be from specific age subgroups, such as those over the age of 1, over the age of 2, over the age of 3, over the age of 4, over the age of 5, over the age of 6, over the age of 7, over the age of 8, over the age of 9, over the age of 10, over the age of 15, over the age of 20, over the age of 25, over the age of 30, over the age of 35, over the age of 40, over the age of 45, over the age of 50, over the age of 55, over the age of 60, over the age of 65, over the age of 70, over the age of 75, over the age of 80, or over the age of 85. Other embodiments of the disclosure pertain to other age groups, such as subjects aged less than age 85, less than age 80, less than age 75, less than age 70, less than age 65, less than age 60, less than age 55, less than age 50, less than age 45, less than age 40, less than age 35, less than age 30, less than age 25, less than age 20, less than age 15, less than age 10, less than age 9, less than age 8, less than age 6, less than age 5, less than age 4, less than age 3, less than age 2, or less than age 1. Other embodiments relate to subjects with age at onset of the disease in any of particular age or age ranges defined by the numerical values described in the above or other numerical values bridging these numbers. It is also contemplated that a range of ages can be relevant in certain embodiments, such as age at onset at more than age 15 but less than age 20. Other age ranges are however also contemplated, including all age ranges bracketed by the age values listed in the above.

The genetic variations of the present disclosure can show an association in other human populations. Particular embodiments comprising subject human populations are thus also contemplated and within the scope of the disclosure. Such embodiments relate to human subjects that are from one or more human populations including, but not limited to, Caucasian, European, American, Eurasian, Asian, Central/South Asian, East Asian, Middle Eastern, African, Hispanic, and Oceanic populations. European populations include, but are not limited to, Swedish, Norwegian, Finnish, Russian, Danish, Icelandic, Irish, Celt, English, Scottish, Dutch, Belgian, French, German, Spanish, Portuguese, Italian, Polish, Bulgarian, Slavic, Serbian, Bosnian, Czech, Greek, and Turkish populations. The racial contribution in subjects can also be determined by genetic analysis, for example, genetic analysis of ancestry can be carried out using unlinked microsatellite markers such as those set out in Smith et al. (Am J Hum Genet 74, 1001-13 (2004)).

It is also well known to the person skilled in the art that certain genetic variations have different population frequencies in different populations, or are polymorphic in one population but not in another. A person skilled in the art can however apply the methods available and as taught herein to practice the present disclosure in any given human population. This can include assessment of genetic variations of the present disclosure, so as to identify those markers that give strongest association within the specific population. Thus, the at-risk variants of the present disclosure can reside on different haplotype backgrounds and in different frequencies in various human populations.

### Samples

Samples that are suitable for use in the methods described herein can be samples from a subject. A sample can be a mammalian tissue or derived therefrom. A sample can be a human tissue or derived therefrom, for example brain tissue (e.g., SN, cortex, brainstem), cells derived from brain meninges, cells derived from human skin fibroblasts. A sample can comprise a nucleic acid. In some cases, a nucleic acid comprising a nucleic acid can include RNA, DNA, polypeptides, or a combination thereof. Nucleic acids and polypeptides can be extracted from one or more samples including but not limited to, blood, saliva, urine, mucosal scrapings of the lining of the mouth, expectorant, serum, tears, skin, tissue, or hair. A sample can be assayed for nucleic acid information. “Nucleic acid information,” as used herein, includes a nucleic acid sequence itself, the presence/absence of genetic variation in the nucleic acid sequence, a physical property which varies depending on the nucleic acid sequence (for example, Tm), and the amount of the nucleic acid (for example, number of mRNA copies). A “nucleic acid” means any one of DNA, RNA, DNA including artificial nucleotides, or RNA including artificial nucleotides. A “recombinant” nucleic acid molecule includes a nucleic acid molecule made by an artificial combination of two otherwise separated segments of sequence, e.g., by chemical synthesis or by the manipulation of isolated segments of nucleic acids by genetic engineering techniques. As used herein, a “polypeptide” includes proteins, fragments of proteins, and peptides, whether isolated from natural sources, produced by recombinant techniques, or chemically synthesized. A polypeptide may have one or more modifications, such as a post-translational modification (e.g., glycosylation, etc.) or any other modification (e.g., pegylation, etc.). The polypeptide may contain one or more non-naturally-occurring amino acids (e.g., such as an amino acid with a side chain modification).

A sample can be processed for RNA or DNA isolation, for example, RNA or DNA in a cell or tissue sample can be separated from other components of the nucleic acid sample. Cells can be harvested from a nucleic acid sample using standard techniques known in the art, for example, by centrifuging a cell sample and resuspending the pelleted cells, for example, in a buffered solution, for example, phosphate-buffered saline (PBS). In some cases, after centrifuging the cell suspension to obtain a cell pellet, the cells can be lysed to extract DNA. In some cases, the sample can be concentrated and/or purified to isolate DNA. All samples obtained from a subject, including those subjected to any sort of further processing, are considered to be obtained from the subject. In some cases, standard techniques and kits known in the art can be used to extract RNA or DNA from a sample, including, for example, phenol extraction, a QIAamp® Tissue Kit (Qiagen, Chatsworth, Calif.), a Wizard® Genomic DNA purification kit (Promega), or a Qiagen Autopure method using Puregene chemistry, which can enable purification of highly stable DNA well-suited for archiving.

Determining the identity of an allele or determining copy number can, but need not, include obtaining a sample comprising RNA and/or DNA from a subject, and/or assessing the identity, copy number, presence or absence of one or more genetic variations, and their chromosomal locations in the nucleic acid sample. The individual or organization that performs the determination need not actually carry out the physical analysis of a sample from a subject. In some cases, the methods can include using information obtained by analysis of sample by a third party. In some cases, the methods can include steps that occur at more than one site. For example, a sample can be obtained from a subject at a first site, such as at a health care provider or at the subject's home in the case of a self-testing kit. The sample can be analyzed at the same or a second site, for example, at a laboratory or other testing facility.

### Methods of Screening

As used herein, “screening” a subject includes diagnosing, theranosing, or determining the susceptibility to developing (prognosing) a neurological disorder, for example, MSA. In particular embodiments, the disclosure is a method of determining a presence of, or a susceptibility to, a neurological disorder, by detecting at least one genetic variation in a nucleic acid sample from a subject as described herein. Detection of particular alleles, markers, variations, or haplotypes is indicative of a presence or susceptibility to a neurological disorder.

Particular genetic variations are found more frequently in individuals with a neurological disorder, than in individuals without screening of a neurological disorder. Therefore, these genetic variations can have predictive value for detecting a neurological disorder, or a susceptibility to a neurological disorder, in an individual. Without intending to be limited, the genetic variations described herein can be associated with susceptibility of a neurological disorder and can represent functional variants predisposing to the disease. A genetic variation can confer a susceptibility of the condition, for example, carriers of the genetic variation are at a different risk of the condition than non-carriers. The presence of a genetic variation can be indicative of increased susceptibility to a neurological disorder, such as MSA. The presence of a genetic variation can be indicative of having a neurological disorder, such as MSA.

Screening can be performed using any of the methods disclosed, alone or in combination. Screening can be performed using Polymerase Chain Reaction (PCR). Screening can be performed using Array Comparative Genomic Hybridization (aCGH). The genetic variation information as it relates to the current disclosure can be used in conjunction with any mentioned symptomatic screening tests to screen a subject for MSA, for example, using a combination of aCGH and different PET radiotracers.

An association with a neurological disorder can determined by the statistical likelihood of the presence of a genetic variation in a subject with a neurological disorder, for example, an unrelated individual or a first or second-degree relation of the subject. An association with a neurological disorder can be determined by determining the statistical likelihood of the absence of a genetic variation in an unaffected reference subject, for example, an unrelated individual or a first or second-degree relation of the subject. The methods described herein can include obtaining and analyzing a nucleic acid sample from one or more suitable reference subjects.

In the present context, the term screening or assessing comprises diagnosis (detecting), prognosis, and theranosis. Screening can refer to any available screening method, including those mentioned herein. As used herein, susceptibility can be proneness of a subject towards the development of a neurological condition, or towards being less able to resist a particular neurological condition than one or more control subjects. Susceptibility can encompass increased susceptibility. For example, particular nucleic acid variations of the disclosure as described herein can be characteristic of increased susceptibility to development of a neurological disorder. Susceptibility can encompass decreased susceptibility, for example, particular nucleic variations of the disclosure as described herein can be characteristic of decreased susceptibility to development of a neurological disorder.

In some cases, an LRRK2 L153L, N551K, I723V, L953L, R1398H, K1423K, R1514Q, P1542S, G1624G, K1637K, M1646T, S1647T, G1819G, N2081D, E2108E, G2385G, or M2397T mutation can decrease susceptibility to development of a neurological disorder. In other cases, an LRRK2 L153L, N551K, I723V, L953L, R1398H, K1423K, R1514Q, P1542S, G1624G, K1637K, M1646T, 51647T, G1819G, N2081D, E2108E, G2385G, or M2397T mutation can increase susceptibility to development of a neurological disorder.

As described herein, a genetic variation predictive of susceptibility to or presence of a neurological disorder can be one where the particular genetic variation is more frequently present in a subject with the condition (affected), compared to the frequency of its presence in a reference group (control), such that the presence of the genetic variation is indicative of susceptibility to or presence of the neurological disorder. The reference group can be a population sample, for example, a random sample from the general population or a mixture of two or more samples from a population. In one aspect, disease-free controls can be characterized by the absence of one or more specific disease-associated symptoms or genetic variation, for example, individuals who have not experienced symptoms associated with a neurological disorder. The disease-free control group is characterized by the absence of one or more disease-specific risk factors, for example, at least one genetic and/or environmental risk factor. A reference sequence can be referred to for a particular site of genetic variation. A reference allele can be a wild-type allele and can be chosen as either the first sequenced allele or as the allele from a control individual. One or more reference subjects can be characteristically matched with one or more affected subjects, for example, with matched aged, gender, or ethnicity.

The disclosure presents a method of screening a subject for a disease or disorder can comprise assaying a nucleic acid sample from the subject to detect sequence information for more than one genetic locus and comparing the sequence information to a panel of nucleic acid biomarkers and screening the subject for the presence or absence of the disease or disorder if one or more of low frequency biomarkers in the panel are present in the sequence information.

The panel can comprise at least one nucleic acid biomarker for each of the more than one genetic loci. For example, the panel can comprise 2, 3, 4, 5, 6, 7, 8, 9, 10, 11, 12, 3, 14, 15, 15, 17, 18, 19, 20, 25, 30, 35, 40, 45, 50, 75, 100, 150, 200, or more nucleic acid biomarkers for each of the more than one genetic loci. The panel can comprise from about 2-1000 nucleic acid biomarkers.

The panel can comprise at least 2 low frequency biomarkers. For example, the panel can comprise at least 2, 3, 4, 5, 6, 7, 8, 9, 10, 11, 12, 3, 14, 15, 15, 17, 18, 19, 20, 25, 30, 35, 40, 45, 50, 55, 60, 65, 70, 75, 80, 85, 90, 95, 100, 125, 150, 175, 200, 250, 500, or 1000 or more low frequency biomarkers. The panel can comprise from about 2-1000 low frequency biomarkers. A low frequency biomarker can occur at a frequency of 0.1% or less in a population of subjects without a diagnosis of the disease or disorder. For example, a low frequency biomarker can occur at a frequency of 0.05%, 0.01%, 0.005%, 0.001%, 0.0005%, 0.0001%, 0.00005%, or 0.00001% or less in a population of subjects without a diagnosis of the disease or disorder. A low frequency biomarker can occur at a frequency from about 0.00001% -0.1% in a population of subjects without a diagnosis of the disease or disorder. For example, a low frequency biomarker can occur at a frequency of from about 0.00001% to about 0.00005%, 0.00001%to about 0.0001%, 0.00001% to about 0.0005%, 0.00001% to about 0.001%, 0.00001% to about 0.005%, 0.00001% to about 0.01%, 0.00001% to about 0.05%, 0.00005%to about 0.0001%, 0.00005% to about 0.0005%, 0.00005% to about 0.001%, 0.00005% to about 0.005%, 0.00005% to about 0.01%, 0.00005% to about 0.05%, 0.00005% to about 0.1%, 0.0001% to about 0.0005%, 0.0001% to about 0.001%, 0.0001% to about 0.005%, 0.0001% to about 0.01%, 0.0001% to about 0.05%, 0.0001% to about 0.1%, 0.0005% to about 0.001%, 0.0005% to about 0.005%, 0.0005% to about 0.01%, 0.0005% to about 0.05%, 0.0005% to about 0.1%, 0.001% to about 0.005%, 0.001% to about 0.01%, 0.001% to about 0.05%, 0.001% to about 0.1%, 0.005% to about 0.01%, 0.005% to about 0.05%, 0.005% to about 0.1%, 0.01% to about 0.05%, 0.01% to about 0.1%, or 0.05%to about 0.1% in a population of subjects without a diagnosis of the disease or disorder.

The presence or absence of the disease or disorder in the subject can be determined with at least 50% confidence. For example, the presence or absence of the disease or disorder in the subject can be determined with at least 50%, 55%, 60%, 65%, 70%, 75%, 80%, 85%, 90%, 95%, 98%, 99%, or 100% confidence. In one aspect, the presence or absence of the disease or disorder in the subject can be determined with a from 50% to 100% confidence.

The present disclosure also pertains to methods of clinical screening, for example, diagnosis, prognosis, or theranosis of a subject performed by a medical professional using the methods disclosed herein. In other embodiments, the disclosure pertains to methods of screening performed by a layman. The layman can be a customer of a genotyping service. The layman can also be a genotype service provider, who performs genotype analysis on a nucleic acid sample from an individual, in order to provide service related to genetic risk factors for particular traits or diseases, based on the genotype status of the subject obtained from use of the methods described herein. The resulting genotype information can be made available to the individual and can be compared to information about neurological disorder or risk of developing a neurological disorder associated with various genetic variations, including but not limited to, information from public literature and scientific publications. The screening applications of neurological disorder-associated genetic variations, as described herein, can, for example, be performed by an individual, a health professional, or a third party, for example, a service provider who interprets genotype information from the subject.

The information derived from analyzing sequence data (for example nucleic acid sequence) can be communicated to any particular body, including the individual from which the sample or sequence data is derived, a guardian or representative of the individual, clinician, research professional, medical professional, service provider, and medical insurer or insurance company. Medical professionals can be, for example, doctors, nurses, medical laboratory technologists, and pharmacists. Research professionals can be, for example, principle investigators, research technicians, postdoctoral trainees, and graduate students.

A professional can be assisted by determining whether specific genetic variants are present in a sample from a subject, and communicating information about genetic variants to a professional. After information about specific genetic variants is reported, a medical professional can take one or more actions that can affect subject care. For example, a medical professional can record information in the subject's medical record regarding the subject's risk of developing a neurological disorder. In one aspect, a medical professional can record information regarding risk assessment, or otherwise transform the subject's medical record, to reflect the subject's current medical condition. In one aspect, a medical professional can review and evaluate a subject's entire medical record and assess multiple treatment strategies for clinical intervention of a subject's condition.

A medical professional can initiate or modify treatment after receiving information regarding a subject's screening of a neurological disorder, for example. A medical professional can recommend a change in therapy. A medical professional can enroll a subject in a clinical trial based on a genetic variation. A subject can be enrolled or not be enrolled in a clinical trial based on a genetic variation.

A medical professional can communicate information regarding a subject's screening of developing a neurological disorder to a subject or a subject's family. A medical professional can provide a subject and/or a subject's family with information regarding a neurological disorder and risk assessment information, including treatment options, and referrals to specialists. A medical professional can provide a copy of a subject's medical records to a specialist. In one aspect, a research professional can apply information regarding a subject's risk of developing a neurological disorder to advance scientific research. In one aspect, a research professional can evaluate a subject's enrollment, or continued participation, in a research study or clinical trial. In one aspect, a research professional can communicate information regarding a subject's screening of a neurological disorder to a medical professional. In one aspect, a research professional can refer a subject to a medical professional.

Also provided herein are databases that include a list of genetic variations as described herein. The list can be stored, for example, on a flat file or computer-readable medium. The databases can further include information regarding one or more subjects, for example, whether a subject is affected or unaffected, clinical information such as endophenotype, age of onset of symptoms, any treatments administered and outcomes, for example, data relevant to pharmacogenomics, diagnostics, prognostics, or theranostics, and other details, for example, data about the disorder in the subject, or environmental or other genetic factors.

The methods described herein can also include the generation of reports for use, for example, by a subject, care giver, or researcher, that include information regarding a subject's genetic variations, and optionally further information such as treatments administered, treatment history, medical history, predicted response, and actual response. The reports can be recorded in a tangible medium, e.g., a computer-readable disk, a solid state memory device, or an optical storage device. Methods of Screening Using Variations in Polypeptides

Screening of a neurological disorder can be made by examining or comparing changes in expression, localization, binding partners, and composition of a polypeptide encoded by a nucleic acid associated with a neurological disorder, for example, in those instances where the genetic variations of the present disclosure results in a change in the composition or expression of the polypeptide and/or RNA, for example, mRNAs, miRNAs, and other noncoding RNAs (ncRNAs). Thus, screening of a neurological disorder can be made by examining expression and/or composition of one of these polypeptides and/or RNA, or another polypeptide and/or RNA encoded by a nucleic acid associated with a neurological disorder, in those instances where the genetic variation of the present disclosure results in a change in the expression, localization, binding partners, and/or composition of the polypeptide and/or RNA. Screening can comprise diagnosing a subject. Screening can comprise determining a prognosis of a subject, for example, determining the susceptibility of developing a neurological disorder. Screening can comprise theranosing a subject.

The genetic variations described herein that show association to a neurological disorder can play a role through their effect on one or more of these nearby genes. For example, while not intending to be limited, it is generally expected that a deletion of a chromosomal segment comprising a particular gene, or a fragment of a gene, can either result in an altered composition or expression, or both, of the encoded polypeptide and/or mRNA. Likewise, duplications, or high number copy number variations, are in general expected to result in increased expression of encoded polypeptide and/or RNA. Other possible mechanisms affecting genes within a genetic variation region include, for example, effects on transcription, effects on RNA splicing, alterations in relative amounts of alternative splice forms of mRNA, effects on RNA stability, effects on transport from the nucleus to cytoplasm, and effects on the efficiency and accuracy of translation. Thus, DNA variations can be detected directly, using the subjects unamplified or amplified genomic DNA, or indirectly, using RNA or DNA obtained from the subject's tissue(s) that are present in an aberrant form or expression level as a result of the genetic variations of the disclosure showing association to a neurological disorder.

Genetic variations of the disclosure showing association to a neurological disorder can affect polypeptide expression at the translational level. It can be appreciated by those skilled in the art that this can occur by increased or decreased expression of one or more microRNAs (miRNAs) that regulates expression of a polypeptide known to be important, or implicated, in the cause, onset, or progression of the neurological disease. Increased or decreased expression of the one or more miRNAs can result from gain or loss of the whole miRNA gene, disruption of a portion of the gene (e.g., by an indel or CNV), or even a single base change (SNP or SNV) that produces an altered, non-functional or aberrant functioning miRNA sequence. It can also be appreciated by those skilled in the art that the expression of polypeptide, for example, one known to cause a neurological disease by increased or decreased expression, can result due to a genetic variation that results in alteration of an existing miRNA binding site within the polypeptide's mRNA transcript, or even creates a new miRNA binding site that leads to aberrant polypeptide expression.

A variety of methods can be used for detecting polypeptide composition and/or expression levels, including but not limited to enzyme linked immunosorbent assays (ELISA), Western blots, spectroscopy, mass spectrometry, peptide arrays, colorimetry, electrophoresis, isoelectric focusing, immunoprecipitations, immunoassays, and immunofluorescence and other methods well-known in the art.

A test sample from a subject can be assessed for the presence of an alteration in the expression and/or an alteration in composition of the polypeptide encoded by a nucleic acid associated with a neurological disorder. An “alteration” in the polypeptide expression or composition, as used herein, refers to an alteration in expression or composition in a test sample, as compared to the expression or composition of the polypeptide in a control sample. Such alteration can, for example, can be an alteration in the quantitative polypeptide expression or can be an alteration in the qualitative polypeptide expression, for example, expression of a mutant polypeptide or of a different splicing variant, or a combination thereof. In some embodiments, screening of a neurological disorder can be made by detecting a particular splicing variant encoded by a nucleic acid associated with a neurological disorder, or a particular pattern of splicing variants. In some embodiments, an antibody can be used to detect the presence or absence of a mutated polypeptide.

Antibodies can be polyclonal or monoclonal and can be labeled or unlabeled. An intact antibody or a fragment thereof can be used. The term “labeled,” with regard to the probe or antibody, is intended to encompass direct labeling of the probe or antibody by coupling a detectable substance to the probe or antibody, as well as indirect labeling of the probe or antibody by reactivity with another reagent that is directly labeled as previously described herein. Other non-limiting examples of indirect labeling include detection of a primary antibody using a labeled secondary antibody, for example, a fluorescently-labeled secondary antibody and end-labeling of a DNA probe with biotin such that it can be detected with fluorescently-labeled streptavidin. A label can be fluorescent or luminescent tags, metals, dyes, radioactive isotopes, and the like. Examples of labels include paramagnetic ions, radioactive isotopes; fluorochromes, metals, dyes, NMR-detectable substances, and X-ray imaging compounds. Paramagnetic ions include chromium (III), manganese (II), iron (III), iron (II), cobalt (II), nickel (II), copper (II), neodymium (II), samarium (III), ytterbium (III), gadolinium (III), vanadium (II), terbium (III), dysprosium (III), holmium (III) and/or erbium (III). Ions useful in other contexts, such as X-ray imaging, include but are not limited to lanthanum (III), gold (III), lead (II), and especially bismuth (III). Radioactive isotopes include ¹⁴-carbon, ¹⁵chromium, ³⁶-chlorine, ⁵⁷cobalt, and the like may be utilized. Among the fluorescent labels contemplated for use include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

### Nucleic Acids

The nucleic acids and polypeptides described herein can be used in methods and kits of the present disclosure. In one aspect, aptamers that specifically bind the nucleic acids or polypeptides described herein can be used in methods and kits of the present disclosure. As used herein, a nucleic acid can comprise a deoxyribonucleotide (DNA) or ribonucleotide (RNA), whether singular or in polymers, naturally occurring or non-naturally occurring, double-stranded or single-stranded, coding, for example, a translated gene, or non-coding , for example, a regulatory region, or any fragments, derivatives, mimetics or complements thereof. Nucleic acids can comprise oligonucleotides, nucleotides, polynucleotides, nucleic acid sequences, genomic sequences, antisense nucleic acids, DNA regions, probes, primers, genes, regulatory regions, introns, exons, open-reading frames, binding sites, target nucleic acids, and allele-specific nucleic acids.

A “probe,” as used herein, can include a nucleic acid fragment for examining a nucleic acid in a specimen using the hybridization reaction based on the complementarity of nucleic acid.

Nucleic acids can be fused to other coding or regulatory sequences can be considered isolated. For example, recombinant DNA contained in a vector is included in the definition of “isolated” as used herein. Isolated nucleic acids can include recombinant DNA molecules in heterologous host cells or heterologous organisms, as well as partially or substantially purified DNA molecules in solution. Isolated nucleic acids also encompass in vivo and in vitro RNA transcripts of the DNA molecules of the present disclosure. An isolated nucleic acid molecule or nucleotide sequence can be synthesized chemically or by recombinant means. Such isolated nucleotide sequences can be useful, for example, in the manufacture of the encoded polypeptide, as probes for isolating homologous sequences (e.g., from other mammalian species), for gene mapping (e.g., by in situ hybridization with chromosomes), or for detecting expression of the gene, in tissue (e.g., human tissue), such as by Northern blot analysis or other hybridization techniques disclosed herein. The disclosure also pertains to nucleic acid sequences that hybridize under high stringency hybridization conditions, such as for selective hybridization, to a nucleotide sequence described herein. Such nucleic acid sequences can be detected and/or isolated by allele-or sequence-specific hybridization (e.g., under high stringency conditions). Stringency conditions and methods for nucleic acid hybridizations are well known to the skilled person (see, e.g., Current Protocols in Molecular Biology, Ausubel, F. et al., John Wiley & Sons, (1998), and Kraus, M. and Aaronson, S., Methods Enzymol., 200:546-556 (1991), the entire teachings of which are incorporated by reference herein.

Calculations of “identity” or “percent identity” between two or more nucleotide or amino acid sequences can be determined by aligning the sequences for optimal comparison purposes (e.g., gaps can be introduced in the sequence of a first sequence). The nucleotides at corresponding positions are then compared, and the percent identity between the two sequences is a function of the number of identical positions shared by the sequences (i.e., % identity=# of identical positions/total# of positions×100). For example, if a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90-5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. The percent identity between two amino acid sequences can be accomplished using, for example, the GAP program in the GCG software package (Accelrys, Cambridge, UK).

Probes can be primers. Primers can be oligonucleotides that hybridize in a base-specific manner to a complementary strand of a nucleic acid molecule. Probes can be labeled as disclosed herein. Probes can include primers, which can be a single-stranded oligonucleotide probe that can act as a point of initiation of template-directed DNA synthesis using methods including but not limited to, polymerase chain reaction (PCR) and ligase chain reaction (LCR) for amplification of a target sequence. Oligonucleotides, as described herein, can include segments or fragments of nucleic acid sequences, or their complements. DNA segments can be between 5 and 10,000 contiguous bases, and can range from 5, 10, 12, 15, 20, or 25 nucleotides to 10, 15, 20, 25, 30, 40, 50, 100, 200, 500, 1000, or 10,000 nucleotides. In addition to DNA and RNA, probes and primers can include polypeptide nucleic acids (PNA), as described in Nielsen, P. et al., Science 254: 1497-1500 (1991). A probe or primer can comprise a region of nucleotide sequence that hybridizes to at least about 10, 11, 12, 13, 14, or 15, typically about 20-25, and in certain embodiments about 40, 50, or 75, consecutive nucleotides of a nucleic acid molecule. In one aspect, primers disclosed herein can share at least 10%, 15%, 20%, 30%, 40% 45%, 50% ,60%, 65%, 70%, 75%, 80%, 85%, 90%, 95%, or 100% identity with the primers disclosed in Table 2.

Nucleosides and derivatives thereof can be used as the building blocks of the primers described herein, except where indicated otherwise. Nothing in this application is meant to preclude the utilization of nucleoside derivatives or bases that have been chemical modified to enhance their stability or usefulness in an amplification reaction, provided that the chemical modification does not interfere with their recognition by a polymerase as deoxyguanine, deoxycytosine, deoxythymidine, or deoxyadenine, as appropriate. Nucleotide analogs can stabilize hybrid formation. In one aspect, nucleotide analogs can destabilize hybrid formation. In one aspect, nucleotide analogs can enhance hybridization specificity. In one aspect, nucleotide analogs can reduce hybridization specificity.

The present disclosure also provides isolated nucleic acids, for example, probes or primers, that contain a fragment or portion that can selectively hybridize to a nucleic acid that comprises, or consists of, a nucleotide sequence, wherein the nucleotide sequence can comprise at least one polymorphism or polymorphic allele contained in the genetic variations described herein or the wild-type nucleotide that is located at the same position, or the compliments thereof. A probe or primer can be at least 70% identical, at least 80% identical, at least 85% identical, at least 90% identical, or at least 95% identical, to a contiguous nucleotide sequence or to a complement of the contiguous nucleotide sequence.

A nucleic acid probe can be an oligonucleotide capable of hybridizing with a complementary regions of a gene associated with a neurological disorder containing a genetic variation described herein. The nucleic acid fragments of the disclosure can be used as probes or primers in assays such as those described herein.

The nucleic acids of the disclosure, such as those described above, can be identified and isolated using standard molecular biology techniques well known to the skilled person. DNA can be amplified and/or can be labeled (e.g., radiolabeled, fluorescently labeled) and used as a probe for screening, for example, a cDNA library derived from an organism. cDNA can be derived from mRNA and can be contained in a suitable vector. For example, corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced, and further characterized.

Nucleic acid can comprise one or more polymorphisms, variations, or mutations, for example, single nucleotide polymorphisms (SNPs), copy number variations (CNVs), for example, insertions, deletions, inversions, and translocations. In one aspect, a nucleic acid may be naturally or non-naturally polymorphic, for example, having one or more sequence differences, for example, additions, deletions and/or substitutions, as compared to a reference sequence. A reference sequence can be based on publicly available information, for example, the U.C. Santa Cruz Human Genome Browser Gateway (genome.ucsc.edu/cgi-bin/hgGateway) or the NCBI website (www.ncbi.nlm.nih.gov). A reference sequence can be determined by a practitioner of the present disclosure using methods well known in the art, for example, by sequencing a reference nucleic acid.

A probe can hybridize to an allele, SNP, or CNV as described herein. A probe can bind to another marker sequence associated with a neurological disorder as described herein.

One of skill in the art would know how to design a probe so that sequence specific hybridization can occur only if a particular allele is present in a genomic sequence from a test nucleic acid sample. The disclosure can also be reduced to practice using any convenient genotyping method, including commercially available technologies and methods for genotyping particular genetic variations.

Control probes can also be used, for example, a probe that binds a less variable sequence, for example, a repetitive DNA associated with a centromere of a chromosome, can be used as a control. In one aspect, probes can be obtained from commercial sources. Probes can be synthesized, for example, chemically or in vitro, or made from chromosomal or genomic DNA through standard techniques. In one aspect sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification using PCR.

One or more nucleic acids, for example a probe or primer, can also be labeled, for example by direct labeling, to comprise a detectable label. A detectable label can comprise any label capable of detection by a physical, chemical, or a biological process for example, a radioactive label, such as 32P or 3H, a fluorescent label, such as FITC, a chromophore label, an affinity-ligand label, an enzyme label, such as alkaline phosphatase, horseradish peroxidase, or 12 galactosidase, an enzyme cofactor label, a hapten conjugate label, such as digoxigenin or dinitrophenyl, a Raman signal generating label, a magnetic label, a spin label, an epitope label, such as the FLAG or HA epitope, a luminescent label, a heavy atom label, a nanoparticle label, an electrochemical label, a light scattering label, a spherical shell label, semiconductor nanocrystal label, such as quantum dots (described in U.S. Pat. No. 6,207,392), and probes labeled with any other signal generating label known to those of skill in the art, wherein a label can allow the probe to be visualized with or without a secondary detection molecule. A nucleotide can be directly incorporated into a probe with standard techniques, for example, nick translation, random priming, and PCR labeling. A “signal,” as used herein, includes a signal suitably detectable and measurable by appropriate means, including fluorescence, radioactivity, chemiluminescence, and the like.

Non-limiting examples of label moieties useful for detection include, without limitation, suitable enzymes such as horseradish peroxidase, alkaline phosphatase, beta-galactosidase, or acetylcholinesterase; members of a binding pair that are capable of forming complexes such as streptavidin/biotin, avidin/biotin, or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include 14C, 1231, 1241, 1251, Tc99m, 32P, 33P, 35S or 3H.

Fluorophores of different colors can be chosen. Fluorescently labeled probes can be viewed with a fluorescence microscope and an appropriate filter for each fluorophore, or by using dual or triple band-pass filter sets to observe multiple fluorophores. Techniques such as flow cytometry can be used to examine the hybridization pattern of the probes.

In other embodiments, a probe can be indirectly labeled, for example, with biotin or digoxygenin, or labeled with radioactive isotopes such as 32P and/or 3H. As a non-limiting example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected using colorimetric reactions using a substrate and/or a catalyst for the enzyme. In one aspect, catalysts for alkaline phosphatase can be used, for example, 5-bromo-4-chloro-3-indolylphosphate and nitro blue tetrazolium. In one aspect, a catalyst can be used for horseradish peroxidase, for example, diaminobenzoate.

### Methods of Detecting Genetic Variations

Standard techniques for genotyping for the presence of genetic variations, for example, amplification, can be used. Amplification of nucleic acids can be accomplished using methods known in the art. Generally, sequence information from the region of interest can be used to design oligonucleotide primers that can be identical or similar in sequence to opposite strands of a template to be amplified. Amplification methods can include, but are not limited to, fluorescence-based techniques utilizing PCR, for example, ligase chain reaction (LCR), Nested PCR, transcription amplification, self-sustained sequence replication, nucleic acid based sequence amplification (NASBA), and multiplex ligation-dependent probe amplification (MLPA). Guidelines for selecting primers for PCR amplification are well known in the art. In some cases, a computer program can be used to design primers, for example, Oligo (National Biosciences, Inc., Plymouth Minn.), MacVector (Kodak/IBI), and GCG suite of sequence analysis programs. LRRK2 genetic variations can be detected. Primers disclosed in Table 2. can be used to detect an LRRK2 genetic variation.

TABLE 2

| rs Number | Amino Acid | PCP Primer Forward | PCR Primer Reverse |
|---|---|---|---|
| rs11175964 | K1423K | ACGTTGGATGCCATTTTATGACGCAGCGAG | ACGTTGGATGGGCTTCATGGCATCAACTTC |
| rs79996240 | K544E | ACGTTGGATGAAACAGTGTTTCAAGAATG | ACGTTGGATGAAAAAGCGCCCCTAGTTCTC |
| rs35847451 | S461S | ACGTTGGATGATTCTACCTGGCCCAATATG | ACGTTGGATGATACATTCTCCTGAAGTGGC |
| rs34995376 | R1441H | ACGTTGGATGGGGTTTTGTGTCTTTCCCTC | ACGTTGGATGCATCCAAATGTGTGCCAACG |
| rs17491187 | L1556I | ACGTTGGATGTCCCGTAATTGACCGGAAAC | ACGTTGGATGAAGTGAACTGCGTGAGGAAG |
| | K871E | ACGTTGGATGCTGTCCATAGAAGAGTCAGG | ACGTTGGATGCTCAGGCAGCGATGGAAATT |
| rs75148313 | S973N | ACGTTGGATGACTTCAATCCCATATGAGGC | ACGTTGGATGTGCTGAAAGGTCTAGTGATG |
| rs33962975 | G2385G | ACGTTGGATGGCAAAATAGCCCTGTTGTGG | ACGTTGGATGCTTAAAAAGTGCACGCAGTC |
| rs33995883 | N2081D | ACGTTGGATGGAGAAAACAGAACTTACCAG | ACGTTGGATGGGAGGTAGAATAGTAGAGGG |
| rs72546336 | N363S | ACGTTGGATGTTGTTTTGGCTGGAAGCCTG | ACGTTGGATGTATGAGAGTCCTACCTGCAC |
| rs60545352 | M2408I | ACGTTGGATGACTTTATGGTTCTAGGGAGG | ACGTTGGATGTGAAGGCAGAGGGTTTTCAC |
| rs41286468 | A312A | ACGTTGGATGCAGAGAATGCAGCATTGCAG | ACGTTGGATGATGCCTCTGTAAGAATCCCC |
| | Y2006H | ACGTTGGATGCCCACAATGTGCTGCTTTTC | ACGTTGGATGCATTCTACAGCAGTACTGAG |
| rs72545324 | H1216R | ACGTTGGATGTATTCAGTACCTACCAGGTC | ACGTTGGATGCACTCAAGTCCAAGATGCTG |
| | A716V | ACGTTGGATGCCTCTGTTGCAAGTGTTTTG | ACGTTGGATGCTGCTCCCAATAGAAGCAAG |
| rs78029637 | T2031S | ACGTTGGATGATGAGACAGACCTGATCACC | ACGTTGGATGCAGTACTGCTGTAGAATGGG |
| rs72545338 | R1325Q | ACGTTGGATGGAACTCACCTAAATCTCAAG | ACGTTGGATGATTCGGTTATAAGGCACAGC |
| rs72545335 | S52F | ACGTTGGATGTTCTTTTCCCCACCCACTTG | ACGTTGGATGAAGACGATCAACAGAGGCAC |
| rs111910483 | L1795F | ACGTTGGATGTTTTGGGCCAAGTTGTGGAC | ACGTTGGATGCAGAGTTTCTCCTTCACCAC |
| rs1427253 | G1524G | ACGTTGGATGTTGACAGTGAAAGTGGAAGG | ACGTTGGATGTTTTCCACATCTCTACGCGA |
| rs17519916 | D944Y | ACGTTGGATGACTGAGTGAATCATCTGAAG | ACGTTGGATGTTAATAGGGGCCCATTTTTG |
| rs111341148 | R1067Q | ACGTTGGATGAAACCACTGAGGGTCCAATG | ACGTTGGATGACACATTTGGACTTGCACAG |
| rs35658131 | Y2189C | ACGTTGGATGAGAGCATTTGGATTTACCTC | ACGTTGGATGCAGAGGACAGCTCTCATTTC |
| rs10878371 | G1819G | ACGTTGGATGCTTCCTCTGCTTTCTTCATC | ACGTTGGATGACTCTGTTGAAGAAATGGGC |
| | M1869V | ACGTTGGATGCTTGATTTTGGCTGACCTGC | ACGTTGGATGAGGAGAAACTCTGGAGCTTG |
| | A1442P | ACGTTGGATGCATCCAAATGTGTGCCAACG | ACGTTGGATGGGGTTTTGTGTCTTTCCCTC |
| rs10878307 | I723V | ACGTTGGATGATGTGATGCTAGAGAGAGCG | ACGTTGGATGTTGGCATCTGCTCCCAATAG |
| rs35517158 | K182K | ACGTTGGATGTGCCATGCACTCATTTCCAG | ACGTTGGATGATACCTCTCTCAAACAGCAC |
| rs34410987 | P755L | ACGTTGGATGTCACGAGATCCACTATTCAG | ACGTTGGATGGCCAGTCTCCTAAAAGGAAG |
| rs35507033 | R1514Q | ACGTTGGATGTAGCACTGAATTTGCCAACC | ACGTTGGATGGGAATCAGCTGTCCAACAAC |
| rs113431706 | R1483Q | ACGTTGGATGGCCAAAGCATCAGATTCCTG | ACGTTGGATGAACTCCTGAATAAGCGAGGG |
| | K1468E | ACGTTGGATGCGTTGGCACACATTTGGATG | ACGTTGGATGCCTCGCTTATTCAGGAGTTC |
| rs72546337 | I810V | ACGTTGGATGAAAGGACCAAGCCAAGAAGG | ACGTTGGATGTGGATGTGGCCAACAATAG |
| rs58559150 | Q923H | ACGTTGGATGGGAGAATTTTACCGAGATGC | ACGTTGGATGCTTACCAAGGAATTGGAATG |
| rs17466213 | I1371V | ACGTTGGATGATCTTGGAATGCAAAGTGCC | ACGTTGGATGTCCCACACATTTAGGACGAG |
| rs35801418 | Y1690C | ACGTTGGATGCCCTGAAAGCATGTAAGGTG | ACGTTGGATGTCCCCATTGTGAGAACTCTG |
| rs78365431 | Q1111H | ACGTTGGATGTGAGCTCTTTCTTACCCTTC | ACGTTGGATGTGTACCTGAGAACCTCACTG |
| rs41286468 | L122L | ACGTTGGATGCTGTTTTTAACTCCAGATTG | ACGTTGGATGGGAGGAGATCTAAGGTCTTC |
| rs55783828 | S1007S | ACGTTGGATGGTGAGTGCATTCTGGTGAAG | ACGTTGGATGGATGCCCTAAGCCAGAAATG |
| rs35602796 | M1869T | ACGTTGGATGCTTGATTTTGGCTGACCTGC | ACGTTGGATGAGGAGAAACTCTGGAGCTTG |
| | L2466H* | ACGTTGGATGCAGGCCAGTTTAATATATAG | ACGTTGGATGTAGCCCAATACCAGCATGAC |
| rs11564148 | S16476 | ACGTTGGATGTATTTCGCGTAGAGATGTGG | ACGTTGGATGCTATTGGCAAAGCAATCTGG |
| rs74985840 | A11516 | ACGTTGGATGTTCTGGCACTGAAACTCTCC | ACGTTGGATGCCACATTTCATCCCTATCAG |
| rs34869625 | G2170G | ACGTTGGATGAATGAGAGCTGTCCTCTGTC | ACGTTGGATGGCATGGTTGCTACACATCAC |
| rs3761863 | M2397T | ACGTTGGATGGTATACTTTATGGTTCTAGGG | ACGTTGGATGTGAAGGCAGAGGGTTTTCAC |
| rs28365216 | N238I | ACGTTGGATGACATTGCCACTCATGAGGAC | ACGTTGGATGTGAAAGGAGAACATGGTTAC |
| rs78501232 | E334K | ACGTTGGATGTCTTCTTCCCCCTCATCATC | ACGTTGGATGTCAAGCTGAGACTATTTTC |
| rs10878405 | E2108E | ACGTTGGATGAAAGAATATGGTTGTGCCCC | ACGTTGGATGGCAGAAGTAGGCCTTTCTTG |
| rs11564178 | R1725STOP | ACGTTGGATGGCCTTGTCGCCAATACATTC | ACGTTGGATGCCCTTTAACTTAGGAAGCAG |
| rs35328937 | R521G | ACGTTGGATGTGTGCATTTAGGCATGCCAG | ACGTTGGATGCAAAGCTGCTAGGACCAGTT |
| rs111501952 | V1450I | ACGTTGGATGGGTTTTGTGTCTTTCCCTCC | ACGTTGGATGGCTTTGCGTTGCTTCTCATC |
| rs72546327 | T1410M | ACGTTGGATGATTCTATAGTACTCATCCCC | ACGTTGGATGCTGAGGTCATAGACAGCAAG |
| rs34015634 | I2012T | ACGTTGGATGCATTCTACAGCAGTACTGAG | ACGTTGGATGCCCACAATGTGCTGCTTTTC |
| rs76535406 | S1096C | ACGTTGGATGCACATCAGTGAGGTTCTCAG | ACGTTGGATGCAGTGAAATGTCCAACTCTG |
| rs60185966 | S1228I | ACGTTGGATGCCGCACACTGGAAATCTTTG | ACGTTGGATGCACTCAAGTCCAAGATGCTG |
| rs33958906 | P1542S | ACGTTGGATGCCGGTCAATTACGGGAAATT | ACGTTGGATGGCTGATTCCAGACTGCTATG |
| rs56105242 | C228S | ACGTTGGATGGGAAATTGTGCTTCATGTGC | ACGTTGGATGTGCTACTTACAAGGAATCGC |
| rs4640000 | P1262A | ACGTTGGATGGTCAGATTTTCAAGACAGCC | ACGTTGGATGTTCTTCCTTCCCACCAACAG |
| rs17480713 | N389N | ACGTTGGATGTCCATGGATGAGAATTCAGC | ACGTTGGATGGCACAGCTTTCACCACAAAC |
| | L1165P | ACGTTGGATGTGTACAACTTAGAGGGCCTG | ACGTTGGATGAAGTGGAGAGTTTCAGTGCC |
| rs11176013 | K1637K | ACGTTGGATGCTATTGGCAAAGCAATCTGG | ACGTTGGATGTATTTCGCGTAGAGATGTGG |
| rs75711334 | L488L | ACGTTGGATGCAGCTGCACTGGTAATGATG | ACGTTGGATGGGATATAATGGCAGCAGTGG |
| | E1874STOP | ACGTTGGATGCTTGATTTTGGCTGACCTGC | ACGTTGGATGAGGAGAAACTCTGGAGCTTG |
| rs10878245 | L153L | ACGTTGGATGCATTGGCTGGAAATGAGTGC | ACGTTGGATGGCAGCTCTTTACTCAGAGCA |
| rs78964014 | E2395K | ACGTTGGATGGAGGGTTTTCACTCTCCCAG | ACGTTGGATGCATTTATCTCTTAATTGGTGG |
| rs7956550 | L953L | ACGTTGGATGCATGAAGATTTACTGAAGCG | ACGTTGGATGACCAAACACTGCATTCTGCC |
| rs78890302 | S827S | ACGTTGGATGTCTCCTTGTTACTTACTTG | ACGTTGGATGGTTGAACCTTCTTGGCTTGG |
| rs111591891 | T2141M | ACGTTGGATGGTAGCAACCATGCATTCAAC | ACGTTGGATGCTCAGGTCTTTGACATTTTG |
| | M712V | ACGTTGGATGGCTGTTATTCTGATCACAC | ACGTTGGATGCCTCTGTTGCAAGTGTTTTG |
| rs12581902 | N2261I | ACGTTGGATGTAACTTGCCATCAGCGGTTC | ACGTTGGATGGTGGAGGAGAACATTAAGGC |
| | E10K | ACGTTGGATGTGGACATTGTTCAGCCTGAC | ACGTTGGATGATGGCTAGTGGCAGCTGTC |
| rs35808385 | L1114L | ACGTTGGATGTGTGGTAGAGAAACTGGAGC | ACGTTGGATGTCAGCATATTTAGGCAACCC |
| rs33939927 | R1441C | ACGTTGGATGCATCCAAATGTGTGCCAACG | ACGTTGGATGGGGTTTTGTGTCTTTCCCTC |
| rs79546190 | V2390M | ACGTTGGATGCTCTGTGACACATGAAGTGC | ACGTTGGATGAACTGAAAAACTCTGTGGGC |
| ss263192805 | R1728H | ACGTTGGATGCCCTTTAACTTAGGAAGCAG | ACGTTGGATGGCCTTGTCGCCAATACATTC |
| rs12423862 | P2119L | ACGTTGGATGAAAGAATATGGTTGTGCCCC | ACGTTGGATGGAATACCTGGGCAGAAGTAG |
| | Q930R | ACGTTGGATGTGTCAGCAGCAAAACACAAC | ACGTTGGATGTTACCGAGATGCCGTATTAC |
| rs28365226 | D1375E | ACGTTGGATGTGGAATGCAAAGTGCCACAG | ACGTTGGATGTCCCACACATTTAGGACGAG |
| rs33949390 | R1628P | ACGTTGGATGGTTGTCCAAAACACCCTAAG | ACGTTGGATGCTATTGGCAAAGCAATCTGG |
| rs33995463 | L119P | ACGTTGGATGACTGACAAGTTTACACTGGC | ACGTTGGATGGCAAATAAGCAAACTTTTGAG |
| rs80179604 | S1228T | ACGTTGGATGCCGCACACTGGAAATCTTTG | ACGTTGGATGCACTCAAGTCCAAGATGCTG |
| rs34637584 | G2019S | ACGTTGGATGTTATCCCCATTCTACAGCAG | ACGTTGGATGCACACTGTATCCCAATGCTG |
| rs34594498 | A419V | ACGTTGGATGCTCTCCATGCTGATGCATTC | ACGTTGGATGGCCCACTGCTTACCATTTTG |
| rs7133914 | R1398H | ACGTTGGATGGGATTCTTGCCTGTCGTTTG | ACGTTGGATGTCGCTGCGTCATAAAATGGG |
| rs7308720 | N551K | ACGTTGGATGATTCACAAACTGGTCCTAGC | ACGTTGGATGCCCCTAGTTCTCCAAATTTA |
| | I1192V | ACGTTGGATGGCCTCCTTCTATGACAATCC | ACGTTGGATGTTCTGTTCCAGCTAATGTGC |
| rs33939927 | R1441G | ACGTTGGATGCATCCAAATGTGTGCCAACG | ACGTTGGATGGGGTTTTGTGTCTTTCCCTC |
| rs77428810 | R1941H | ACGTTGGATGAGGCTAACTCCATCACCAAC | ACGTTGGATGTTGTGGTGCTTTGCCACCTC |
| rs34805604 | I1122V | ACGTTGGATGCTTGTCTCTTGTGACTAGAA | ACGTTGGATGAGTTCCTTCAGTCTCAAGGG |
| | R1728L | ACGTTGGATGCCCTTTAACTTAGGAAGCAG | ACGTTGGATGGCCTTGTCGCCAATACATTC |
| | L1870F | ACGTTGGATGAGGAGAAACTCTGGAGCTTG | ACGTTGGATGCTTGATTTTGGCTGACCTGC |
| rs721710 | V1598E | ACGTTGGATGAAAAGACACCAACCTGTGCC | ACGTTGGATGCAGCACTGCAGTTAAGTGAC |
| rs35870237 | I2020T | ACGTTGGATGCCCACAATGTGCTGCTTTTC | ACGTTGGATGTTATCCCCATTCTACAGCAG |
| rs35303786 | M1646T | ACGTTGGATGCTATGGCAAAGCAATCTGG | ACGTTGGATGTATTTCGCGTAGAGATGTGG |
| rs77018758 | R1320S | ACGTTGGATGGAACTCACCTAAATCTCAAG | ACGTTGGATGGCACAGCCTTTTTTAATCGC |
| rs74163686 | N1437H | ACGTTGGATGGTTGATGCCATGAAGCCTTG | ACGTTGGATGCACACAGACACATGAATTA |
| rs74681492 | P1446L | ACGTTGGATGCATCCAAATGTGTGCCAACG | ACGTTGGATGGGGTTTTGTGTCTTTCCCTC |
| rs35173587 | R793M* | ACGTTGGATGTGCTATTGTTGGCCACATCC | ACGTTGGATGAGCATTGGGAAAGGTGACAG |
| rs111503579 | L1653L | ACGTTGGATGTCTTCTCCTATTGGCAAAGC | ACGTTGGATGGGAAATTTCCAAAGAACTAC |
| rs112794616 | A211V | ACGTTGGATGGGAGCAACTGACTGAATTTG | ACGTTGGATGGCACATGAAGCACAATTTCC |
| rs35363614 | R1462X146B | ACGTTGGATGCCTCGCTTATTCAGGAGTTC | ACGTTGGATGGGCACACATTTGGATGTTTC |
| rs111272009 | T2356fsX2360 | ACGTTGGATGCCACAACAGGGCTATTTTGC | ACGTTGGATGCAGCTTTCAGTGATTCCAAG |
| rs113217062 | I1006M | ACGTTGGATGGATGCCCTAAGCCAGAAATG | ACGTTGGATGGTGAGTGCATTCTGGTGAAG |
| rs72546315 | H275H | ACGTTGGATGATTCAAGAAGTGAGTTGCTG | ACGTTGGATGATCATACAGTGTGTGATGAC |
| rs55633591 | N2479D | ACGTTGGATGTGCTGGTATTGGGCTACAAC | ACGTTGGATGCACATCATTGCCCTGTTTGG |
| rs79909111 | S1721S | ACGTTGGATGGTCTTCATTAGATACTTACC | ACGTTGGATGCCAATGGGATTTTGGTCAAG |
| rs113065049 | V366M | ACGTTGGATGGTTACAAAGCATTAACGTGG | ACGTTGGATGCTCTAATATTTATGAGAGTCC |
| rs78154368 | S663P | ACGTTGGATGGGGATTTCAGACAATCTTAGC | ACGTTGGATGCAAATGAATGATGCACCAGC |
| rs113589830 | D1420N | ACGTTGGATGCCATTTTATGACGCAGCGAG | ACGTTGGATGGGCTTCATGGCATCAACTTC |
| rs2256408 | R50H | ACGTTGGATGTTTGCAAATGTAAGGAGGGG | ACGTTGGATGAGGATCTGCTGGTGTTCACG |
| rs77424631 | G558G | ACGTTGGATGGCATCAGGAAAATGTACAA | ACGTTGGATGAACAGCGAGTATTCTTTTG |
| rs111739194 | L2063STOP | ACGTTGGATGCTATTCTACCTCCAGTTGTC | ACGTTGGATGAAGTTGCCAGAGGAAATGTC |
| rs35611877 | L708fsX718 | ACGTTGGATGTTCTGATCACACGCTCTCTC | ACGTTGGATGCCTCTGTTGCAAGTGTTTTG |
| rs75054132 | A75A | ACGTTGGATGCTGTTGATCGTCTTGGACTC | ACGTTGGATGAGGGAGAATGAGTTGAAGTG |
| rs34778348 | G2385R | ACGTTGGATGGCAAAATAGCCCTGTTGTGG | ACGTTGGATGCTTAAAAAGTGCACGCAGTC |
| rs34090008 | P514fsX529 | ACGTTGGATGGTTATCTCTTCCCACAATC | ACGTTGGATGGGCGCTTCGAGCTATTTTAC |
| rs36016791 | A352fsX357 | ACGTTGGATGCAAGAGAATGATGATGAGGG | ACGTTGGATGCTGCACGTGCTTGTTCTTTC |
| rs11435410 | A1430A | ACGTTGGATGACCTTGCTGTCTATGACCTC | ACGTTGGATGGAACAAATCACCTTTATAT |
| rs57355477 | A298A | ACGTTGGATGGAAGTCCATGAGTTTGTGGT | ACGTTGGATGCTGCAATGCTGCATTCTCTG |
| rs113511708 | T2356I | ACGTTGGATGCAGCTTTCAGTGATTCCAAC | ACGTTGGATGCCACAACAGGGCTATTTTGC |
| | V1613A5 | ACGTTGGATGTATGACTCGAATCTTTCAG | ACGTTGGATGCCCTTAGGGTGTTTTGGAC |
| rs72546319 | V674V | ACGTTGGATGCCTGTTGATCCTTTTGTTCC | ACGTTGGATGAAGCTGCTGGTGCATCATTC |
| rs112998035 | R1441R | ACGTTGGATGGAAACATCCAAATGTGTGCC | ACGTTGGATGAGAGGGTTTTGTGTCTTTCC |
| | rs Number | Amino Acid | Extend Primer |
| | rs11175964 | K1423K | TCAACTTCAGCCTGTCC |
| | rs79996240 | K544E | AGCTGCTAGGACCAGTT |
| | rs35847451 | S461S | CCTGAAGTGGCTGAAAG |
| | rs34995376 | R1441H | ACAGGGGAAGAAGAAGCG |
| | rs17491187 | L1556I | AGCTGATTTTCTCTCACTA |
| | | K871E | acCTGAAGATGTGCTGTCT |
| | rs75148313 | S973N | gGAAGCCAGAGAAGAAATG |
| | rs33962975 | G2385G | AAGTGCACGCAGTCTATTAG |
| | rs33995883 | N2081D | ccGAGGGTTTGAAGTTTCCA |
| | rs72546336 | N363S | cccaCTACCTGCACGTGCTTG |
| | rs60545352 | M2408I | TTTCACTCTCCCAGAATAAGA |
| | rs41286468 | A312A | ggatGAGGGCCAAACAGCTGAG |
| | | Y2006H | ggaggGATGGCAGCATTGGGAT |
| | rs72545324 | H1216R | aCCTTAAGTTCAAAGATTTCCAG |
| | | A716V | agatGCTGTTATTCTGATCACAC |
| | rs78029637 | T2031S | ACTGCTGTAGAATGGGGATAAAA |
| | rs72545338 | R1325Q | gtagAAGGCACAGCCTTTTTTAAT |
| | rs72545335 | S52F | gtagTTTTGCCTTGAAATAACTTG |
| | rs111910483 | L1795F | cgTCACCACAAATATCAATCTCCAG |
| | rs1427253 | G1524G | cttaCACATCTCTACGCGAAATAAT |
| | rs17519916 | D944Y | ggAGGGGCCCATTTTTGATCATGAA |
| | rs111341148 | R1067Q | ttggATTGCTAATCTTGATGTCTCTC |
| | rs35658131 | Y2189C | cttgCTTGACTTAAATACTGAAGGAT |
| | rs10878371 | G1819G | gGGGCATTATATAGTTTTAATGATGG |
| | | M1869V | aaacTTCCAACTCATCATTATTCAACA |
| | | A1442P | TTTCCCTCCAGGCTCGC |
| | rs10878307 | I723V | CAAGCATTCAACCATGA |
| | rs35517158 | K182K | AACAGCACATGTAAAGC |
| | rs34410987 | P755L | cTGAGAAAGAGAGCAGTC |
| | rs35507033 | R1514Q | TCCAACAACAAGCTGATCT |
| | rs113431706 | R1483Q | caAGGGTTCCCTGCCATAC |
| | | K1468E | AGGAGTTCCTTGGTGATTT |
| | rs72546337 | I810V | aaGCCTTGGAGGATTTTGT |
| | rs58559150 | Q923H | ggaAAATTTGGTGAGCAACG |
| | rs17466213 | I1371V | TAGGCCAGTCTTTCACATCTA |
| | rs35801418 | Y1690C | cCCGACTATATGAAATGCCTT |
| | rs78365431 | Q1111H | TGTGGTAGAGAAACTGGAGCA |
| | rs41286468 | L122L | ACACTGGCATTATGAACTGTTA |
| | rs55783828 | S1007S | tgAGCCAGAAATGCTGTATAAG |
| | rs35602796 | M1869T | gTTCCAACTCATCATTATTCAAC |
| | | L2466H* | CCAATACCAGCATGACATTTTTA |
| | rs11564148 | S16476 | gTCTAGGAGCTTAAAATACTGTG |
| | rs74985840 | A11516 | gCCCTATCAGAGAACTTTCTTGAG |
| | rs34869625 | G2170G | gcGGAATGCAAGCATTTGGCTGGG |
| | rs3761863 | M2397T | gTTTGATTCCTTGTTTTCTTTTACC |
| | rs28365216 | N238I | gTGATCTCTTTAAATTCAGGCAATA |
| | rs78501232 | E334K | AAATCAAGATTTAGAGGAAAAGAAT |
| | rs10878405 | E2108E | TCTTTCAAACACTGTTTAATTAATTT |
| | rs11564178 | R1725STOP | gtgagTAATGGCTCCATTTTTTAGAA |
| | rs35328937 | R521G | accTATGATGAAATTCTGTATCCTCCC |
| | rs111501952 | V1450I | ATCCAAATGTGTGCCAA |
| | rs72546327 | T1410M | GGTACAATGCTCGCTGC |
| | rs34015634 | I2012T | cTCCCAATGCTGCCATCA |
| | rs76535406 | S1096C | tTGTCATATAACCAGCTGT |
| | rs60185966 | S1228I | GATGCTGATCTGATTATGG |
| | rs33958906 | P1542S | ggCGGAGCGTAAAAATGTG |
| | rs56105242 | C228S | ggATCGCTAGGGAATGTAAA |
| | rs4640000 | P1262A | TTTTTTCCCATTAAGATTCCT |
| | rs17480713 | N389N | gACCACAAACTCATGGACTTC |
| | | L1165P | TTCAGTGCCAGAATGAATTTTC |
| | rs11176013 | K1637K | cTGTGGAAAAATTTCTTTCAAA |
| | rs75711334 | L488L | ccctCAGTGGTCCCCAAAATACT |
| | | E1874STOP | ccGGAGCTTGTTCAAATTCCAACT |
| | rs10878245 | L153L | ggAGGTAAAATCACCTTGCTGATA |
| | rs78964014 | E2395K | TTAATGTATACTTTATGGTTCTAGG |
| | rs7956550 | L953L | gggaTGAGTGAATCATCTGAAGATA |
| | rs78890302 | S827S | tGTCCTTTATTTCCAGATAAGACTTC |
| | rs111591891 | T2141M | gaagTCAGCTGAATTAGTCTGTCTGA |
| | | M712V | atcTGGATGATTACTTAAAAAATGTG |
| | rs12581902 | N2261I | ccacTTTTCCTTTTAGCAAACAAAAAA |
| | | E10K | ccAGTTTCCTCGTCCTCTT |
| | rs35808385 | L1114L | AGCTCTTTCTTACCCTTC |
| | rs33939927 | R1441C | TGTCTTTCCCTCCAGGCT |
| | rs79546190 | V2390M | aGTGGGCTAATAGACTGC |
| | ss263192805 | R1728H | CCAATACATTCTGTTTGGG |
| | rs12423862 | P2119L | caAAGTAGGCCTTTCTTGA |
| | | Q930R | cctTTGCTCACCAAATTTGC |
| | rs28365226 | D1375E | ggTCTTATTTGGATAGGCCA |
| | rs33949390 | R1628P | AGAAATTTTTCCACATCTCTA |
| | rs33995463 | L119P | GTTTTTAACTCCAGATTGATTC |
| | rs80179604 | S1228T | aAAGATGCTGATCTGATTATGG |
| | rs34637584 | G2019S | ccctTGCAAAGATTGCTGACTAC |
| | rs34594498 | A419V | agagGAGAGTTGACAATGCATTC |
| | rs7133914 | R1398H | ttcTGAGTACTATAGAATTCCTCA |
| | rs7308720 | N551K | ccGAAAACTTATATTCAACATACCCT |
| | | I1192V | gggcACTTACTGTGGAAGATTTAAAA |
| | rs33939927 | R1441G | GTCTTTCCCTCCAGGCT |
| | rs77428810 | R1941H | GCTGGCAGCTGGGATTC |
| | rs34805604 | I1122V | GTCTCAAGGGGGAGCATA |
| | | R1728L | CCAATACATTCTGTTTGGG |
| | | L1870F | CCTGCCTAGAAATATTATGTT |
| | rs721710 | V1598E | aAGTTAAGTGACTTGTACTTTG |
| | rs35870237 | I2020T | CCCATTCTACAGCAGTACTGAGCA |
| | rs35303786 | M1646T | ccacGGAAATTTCCAAAGAACTACA |
| | rs77018758 | R1320S | GCCTTTTTTAATCGCTGTTGAAGAAA |
| | rs74163686 | N1437H | AAGCCTTGGCTCTTC |
| | rs74681492 | P1446L | GCCAACGAGAATCACA |
| | rs35173587 | R793M* | TCAGCTTGCTCTTAAGGA |
| | rs111503579 | L1653L | AAAGCAATCTGGAATTTTTC |
| | rs112794616 | A211V | ACAAAGATTATATGATATTGTTAAGTG |
| | rs35363614 | R1462X146B | ACTCATGCAGGCTTT |
| | rs111272009 | T2356fsX2360 | AGTGTCTACCACCACTG |
| | rs113217062 | I1006M | AAGCCAGAAATGCTGTAT |
| | rs72546315 | H275H | ccctTTGCTGTTTGCTCCA |
| | rs55633591 | N2479D | aagtGGCTACAACCGGAAA |
| | rs79909111 | S1721S | ATTAGATACTTACCTCTCCC |
| | rs113065049 | V366M | GGCATAGAAAGAACAAGCAC |
| | rs78154368 | S663P | CTCAAATTGTCAGCATCTTTT |
| | rs113589830 | D1420N | tCATTGTACCTTGCTGTCTAT |
| | rs2256408 | R50H | GTTTATTTTCAAGTGATTACCG |
| | rs77424631 | G558G | cCTTTTAATCCACATTTCTGAAT |
| | rs111739194 | L2063STOP | CTCCAGTTGTCAAAATGTCATAG |
| | rs35611877 | L708fsX718 | tctaTCTCTAGCATCACATTTTTT |
| | rs75054132 | A75A | ggtgtACTCCTATATGAGAGTCGC |
| | rs34778348 | G2385R | ggtgAGAAAACTGAAAAACTCTGT |
| | rs34090008 | P514fsX529 | cccaACAATCAACTATGTAACTTACC |
| | rs36016791 | A352fsX357 | cGTTTTGGCTGGAAGC |
| | rs11435410 | A1430A | AGGCTGAAGTTGATGC |
| | rs57355477 | A298A | TGAGTTTGTGGTGAAAGC |
| | rs113511708 | T2356I | CAGTGATTCCAACATCATAA |
| | | V1613A5 | GAATCTTTCAGATTTTGACAG |
| | rs72546319 | V674V | GGAAGACATTTGATGGAATAT |
| | rs112998035 | R1441R | ggggcCAGGGGAAGAAGAAGC |

Examples of PCR techniques that can be used in the present disclosure include, but are not limited to, quantitative PCR, real-time quantitative PCR (qPCR), quantitative fluorescent PCR (QF-PCR), multiplex fluorescent PCR (MF-PCR), real time PCR (RT-PCR), single cell PCR, PCR-RFLP/RT-PCR-RFLP, hot start PCR, and Nested PCR. Other suitable amplification methods include the ligase chain reaction (LCR), ligation mediated PCR (LM-PCR), degenerate oligonucleotide probe PCR (DOP-PCR), transcription amplification, self-sustained sequence replication, selective amplification of target polynucleotide sequences, consensus sequence primed polymerase chain reaction (CP-PCR), arbitrarily primed polymerase chain reaction (AP-PCR), and nucleic acid based sequence amplification (NABSA).

Alternative methods for the simultaneous interrogation of multiple regions include quantitative multiplex PCR of short fluorescent fragments (QMPSF), multiplex amplifiable probe hybridization (MAPH), and multiplex ligation-dependent probe amplification (MLPA).

Commercial methodologies available for genotyping, for example, SNP genotyping, can be used, but are not limited to, TaqMan genotyping assays (Applied Biosystems), SNPlex platforms (Applied Biosystems), gel electrophoresis, capillary electrophoresis, size exclusion chromatography, mass spectrometry, for example, MassARRAY system (Sequenom), minisequencing methods, real-time Polymerase Chain Reaction (PCR), Bio-Plex system (BioRad), CEQ and SNPstream systems (Beckman), array hybridization technology, for example, Affymetrix GeneChip (Perlegen), BeadArray Technologies, for example, Illumina GoldenGate and Infinium assays, array tag technology, Multiplex Ligation-dependent Probe Amplification (MLPA), and endonuclease-based fluorescence hybridization technology (Invader; Third Wave). In some cases, real-time quantitative PCR can be used to determine genetic variations, wherein quantitative PCR can permit both detection and quantification of a DNA sequence in a nucleic acid sample, for example, as an absolute number of copies or as a relative amount when normalized to DNA input or other normalizing genes. In some cases, methods of quantification can include the use of fluorescent dyes that can intercalate with double-stranded DNA, and modified DNA oligonucleotide probes that can fluoresce when hybridized with a complementary DNA.

DNA can be amplified on a bead or a solid substrate. In some cases, the amplification on the bead results in each bead carrying at least one million, at least 5 million, or at least 10 million copies of the single amplified piece of DNA molecule.

Where PCR occurs in oil-emulsion mixtures, the emulsion droplets can be broken, the DNA can be denatured, and the beads carrying single-stranded nucleic acids clones can be deposited into a well, such as a picoliter-sized well, for further analysis according to the methods described herein. These amplification methods allow for the analysis of genomic DNA regions. Methods for using bead amplification followed by fiber optics detection are described in Margulies et al. 2005, Nature. 15; 437(7057):376-80, and as well as in US Publication Application Nos. 20020012930; 20030068629; 20030100102; 20030148344; 20040248161; 20050079510, 20050124022; and 20060078909.

Identification of genetic variations can be accomplished using hybridization methods. The presence of a specific marker allele or a particular genomic segment comprising a genetic variation, or representative of a genetic variation, can be indicated by sequence-specific hybridization of a nucleic acid probe specific for the particular allele or the genetic variation in a nucleic acid sample that has or has not been amplified but methods described herein. The presence of more than one specific marker allele or several genetic variations can be indicated by using two or more sequence-specific nucleic acid probes, wherein each is specific for a particular allele and/or genetic variation.

Hybridization can be performed by methods well known to the person skilled in the art, for example, hybridization techniques such as fluorescent in situ hybridization (FISH), Southern analysis, Northern analysis, or in situ hybridization. In some cases, hybridization refers to specific hybridization, wherein hybridization can be performed with no mismatches. Specific hybridization, if present, can use standard methods. In some cases, if specific hybridization occurs between a nucleic acid probe and the nucleic acid in the nucleic acid sample, the nucleic acid sample can contain a sequence that can be complementary to a nucleotide present in the nucleic acid probe. In some cases, if a nucleic acid probe can contain a particular allele of a polymorphic marker, or particular alleles for a plurality of markers, specific hybridization is indicative of the nucleic acid being completely complementary to the nucleic acid probe, including the particular alleles at polymorphic markers within the probe. In some cases a probe can contain more than one marker alleles of a particular haplotype, for example, a probe can contain alleles complementary to 2, 3, 4, 5 or all of the markers that make up a particular haplotype. In some cases detection of one or more particular markers of the haplotype in the nucleic acid sample is indicative that the source of the nucleic acid sample has the particular haplotype.

PCR conditions and primers can be developed that amplify a product only when the variant allele is present or only when the wild type allele is present, for example, allele-specific PCR. In some cases of allele-specific PCR, a method utilizing a detection oligonucleotide probe comprising a fluorescent moiety or group at its 3′ terminus and a quencher at its 5′ terminus, and an enhancer oligonucleotide, can be employed, as described by Kutyavin et al. (Nucleic Acid Res. 34:e128 (2006)).

An allele-specific primer/probe can be an oligonucleotide that is specific for a particular polymorphism can be prepared using standard methods. In some cases, allele-specific oligonucleotide probes can specifically hybridize to a nucleic acid region that contains a genetic variation. In some cases, hybridization conditions can be selected such that a nucleic acid probe can specifically bind to the sequence of interest, for example, the variant nucleic acid sequence.

Allele-specific restriction digest analysis can be used to detect the existence of a polymorphic variant of a polymorphism, if alternate polymorphic variants of the polymorphism can result in the creation or elimination of a restriction site. Allele-specific restriction digests can be performed, for example, with the particular restriction enzyme that can differentiate the alleles. In some cases, PCR can be used to amplify a region comprising the polymorphic site, and restriction fragment length polymorphism analysis can be conducted. In some cases, for sequence variants that do not alter a common restriction site, mutagenic primers can be designed that can introduce one or more restriction sites when the variant allele is present or when the wild type allele is present.

Fluorescence polarization template-directed dye-terminator incorporation (FP-TDI) can be used to determine which of multiple polymorphic variants of a polymorphism can be present in a subject.

DNA containing an amplified portion can be dot-blotted, using standard methods and the blot contacted with the oligonucleotide probe. The presence of specific hybridization of the probe to the DNA can then be detected. The methods can include determining the genotype of a subject with respect to both copies of the polymorphic site present in the genome, wherein if multiple polymorphic variants exist at a site, this can be appropriately indicated by specifying which variants are present in a subject. Any of the detection means described herein can be used to determine the genotype of a subject with respect to one or both copies of the polymorphism present in the subject's genome.

A peptide nucleic acid (PNA) probe can be used in addition to, or instead of, a nucleic acid probe in the methods described herein. A PNA can be a DNA mimic having a peptide-like, inorganic backbone, for example, N-(2-aminoethyl) glycine units with an organic base (A, G, C, T or U) attached to the glycine nitrogen via a methylene carbonyl linker.

Nucleic acid sequence analysis can also be used to detect genetic variations, for example, genetic variations can be detected by sequencing exons, introns, 5′ untranslated sequences, or 3′ untranslated sequences. One or more methods of nucleic acid analysis that are available to those skilled in the art can be used to detect genetic variations, including, but not limited to, direct manual sequencing, automated fluorescent sequencing, single-stranded conformation polymorphism assays (SSCP), clamped denaturing gel electrophoresis (CDGE), denaturing gradient gel electrophoresis (DGGE), two-dimensional gel electrophoresis (2DGE or TDGE), conformational sensitive gel electrophoresis (CSGE), denaturing high performance liquid chromatography (DHPLC), infrared matrix-assisted laser desorption/ionization (IR-MALDI) mass spectrometry, mobility shift analysis, quantitative real-time PCR, restriction enzyme analysis, heteroduplex analysis, chemical mismatch cleavage (CMC), RNase protection assays, use of polypeptides that recognize nucleotide mismatches, allele-specific PCR, real-time pyrophosphate DNA sequencing, PCR amplification in combination with denaturing high performance liquid chromatography (dHPLC), and combinations of such methods.

Sequencing can be performed by any sequencing method known in the art. Sequencing can be performed in high throughput. Suitable next generation sequencing technologies include the 454 Life Sciences platform (Roche, Branford, Conn.) (Margulies et al., Nature, 437, 376-380 (2005)); lllumina's Genome Analyzer, GoldenGate Methylation Assay, or Infinium Methylation Assays, i e , Infinium HumanMethylation 27K BeadArray or VeraCode GoldenGate methylation array (Illumina, San Diego, Calif.; Bibkova et al., Genome Res. 16, 383-393 (2006); and U.S. Pat. Nos. 6,306,597, 7,598,035, 7,232,656), or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453); or the Helicos True Single Molecule DNA sequencing technology (Harris et al., Science, 320, 106-109 (2008); and U.S. Pat. Nos. 7,037,687, 7,645,596, 7,169,560, and7,769,400), the single molecule, real-time (SMRT™) technology of Pacific Biosciences, and sequencing (Soni et al., Clin. Chem. 53, 1996-2001 (2007)). These systems allow multiplexed parallel sequencing of many polynucleotides isolated from a sample (Dear, Brief Funct. Genomic Proteomic, 1(4), 397-416 (2003) and McCaughan et al., J. Pathol., 220, 297-306 (2010)). In some cases, polynucleotides are sequenced by sequencing by ligation of dye-modified probes, pyrosequencing, or single-molecule sequencing. Determining the sequence of a polynucleotide may be performed by sequencing methods such as HeliScope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD™ sequencing, Ion Torrent™, Ion semiconductor sequencing, Single Molecule SMRT™ sequencing, Polony sequencing, DNA nanoball sequencing, and VisiGen Biotechnologies approach. Alternatively, determining the sequence of polynucleotides may use sequencing platforms, including, but not limited to, Genome Analyzer IIx, HiSeq, and MiSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, Mass.). Sequencing can comprise MiSeq sequencing. Sequencing can comprise HiSeq sequencing. Determining the sequence of a polynucleotide can comprise paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof. Alternatively, the sequence of a polynucleotide can be determined by electron microscopy or a chemical-sensitive field effect transistor (chemFET) array.

High-throughput sequencing methods can include but are not limited to, Massively Parallel Signature Sequencing (MPSS, Lynx Therapeutics), Polony sequencing, 454 pyrosequencing, Illumina (Solexa) sequencing, SOLiD™ sequencing, on semiconductor sequencing, DNA nanoball sequencing, HeliScope™ single molecule sequencing, Single Molecule SMRT™ sequencing, Single Molecule real time (RNAP) sequencing, Nanopore DNA sequencing, and/or sequencing by hybridization, for example, a non-enzymatic method that uses a DNA microarray, or microfluidic Sanger sequencing. High-throughput sequencing can involve the use of technology available by Helicos BioSciences Corporation (Cambridge, Mass.) such as the Single Molecule Sequencing by Synthesis (SMSS) method as described in US Publication Application Nos. 20060024711; 20060024678; 20060012793; 20060012784; and 20050100932.

Analysis by restriction enzyme digestion can be used to detect a particular genetic variation if the genetic variation results in creation or elimination of one or more restriction sites relative to a reference sequence. In some cases, restriction fragment length polymorphism (RFLP) analysis can be conducted, wherein the digestion pattern of the relevant DNA fragment indicates the presence or absence of the particular genetic variation in the nucleic acid sample.

Arrays of oligonucleotide probes that can be complementary to target nucleic acid sequence segments from a subject can be used to identify genetic variations. An array of oligonucleotide probes can comprise an oligonucleotide array, for example, a microarray. In some cases, the present disclosure features arrays that include a substrate having a plurality of addressable areas, and methods of using them. At least one area of the plurality includes a nucleic acid probe that binds specifically to a sequence comprising a genetic variation, and can be used to detect the absence or presence of the genetic variation, for example, one or more SNPs, or microsatellites as described herein, to determine or identify an allele or genotype. For example, an array can include one or more nucleic acid probes that can be used to detect a genetic variation associated with a gene and/or gene product such as those associated with LRRK2 or LRRK2. In some cases, the array can further comprise at least one area that includes a nucleic acid probe that can be used to specifically detect another marker associated with a neurological disorder.

Microarray hybridization can be performed by hybridizing a nucleic acid of interest, for example, a nucleic acid encompassing a genetic variation, with the array and detecting hybridization using nucleic acid probes. In some cases, the nucleic acid of interest is amplified prior to hybridization. Hybridization and detecting can be carried out according to standard methods described in Published PCT Applications: WO 92/10092 and WO 95/11995, and U.S. Pat. No. 5,424,186. For example, an array can be scanned to determine the position on the array to which the nucleic acid hybridizes. The hybridization data obtained from the scan can be, for example, in the form of fluorescence intensities as a function of location on the array.

Oligonucleotide probes forming an array can be attached to a substrate by any number of techniques, including, but not limited to, in situ synthesis, for example, high-density oligonucleotide arrays, using photolithographic techniques; spotting/printing a medium to low density on glass, nylon, or nitrocellulose; by masking; and by dot-blotting on a nylon or nitrocellulose hybridization membrane. In some cases, oligonucleotides can be immobilized via a linker, including, but not limited to, by covalent, ionic, or physical linkage. Linkers for immobilizing nucleic acids and polypeptides, including reversible or cleavable linkers, are known in the art (U.S. Pat. No. 5,451,683 and WO98/20019). In some cases, oligonucleotides can be non-covalently immobilized on a substrate by hybridization to anchors, by means of magnetic beads, or in a fluid phase, for example, in wells or capillaries.

An array can comprise oligonucleotide hybridization probes capable of specifically hybridizing to different genetic variations. In some cases, oligonucleotide arrays can comprise a plurality of different oligonucleotide probes coupled to a surface of a substrate in different known locations. In some cases, oligonucleotide probes can exhibit differential or selective binding to polymorphic sites, and can be readily designed by one of ordinary skill in the art, for example, an oligonucleotide that is perfectly complementary to a sequence that encompasses a polymorphic site, for example, a sequence that includes the polymorphic site, within it, or at one end, can hybridize preferentially to a nucleic acid comprising that sequence, as opposed to a nucleic acid comprising an alternate polymorphic variant.

Arrays can include multiple detection blocks, for example, multiple groups of probes designed for detection of particular polymorphisms. In some cases, these arrays can be used to analyze multiple different polymorphisms. In some cases, detection blocks can be grouped within a single array or in multiple, separate arrays, wherein varying conditions, for example, conditions optimized for particular polymorphisms, can be used during hybridization. General descriptions of using oligonucleotide arrays for detection of polymorphisms can be found, for example, in U.S. Pat. Nos. 5,858,659 and 5,837,832. In addition to oligonucleotide arrays, cDNA arrays can be used similarly in certain embodiments.

The methods described herein can include, but are not limited to, providing an array as described herein, contacting the array with a sample, and detecting binding of a nucleic acid from the sample to the array. The method can comprise amplifying nucleic acid from the sample, for example, a region associated with a neurological disorder or a region that includes another region associated with a neurological disorder. The methods described herein can include using an array that can identify differential expression patterns or copy numbers of one or more genes in a samples from control and affected individuals. For example, arrays of probes to a marker described herein can be used to identify genetic variations between DNA from an affected subject, and control DNA obtained from an individual that does not have a neurological disorder. Since the nucleotides on the array can contain sequence tags or labels, their positions on the array can be accurately known relative to the genomic sequence.

It can be desirable to employ methods that can detect the presence of multiple genetic variations, for example, polymorphic variants at a plurality of polymorphic sites, in parallel or substantially simultaneously. In some cases, these methods can comprise oligonucleotide arrays and other methods, including methods in which reactions, for example, amplification and hybridization, can be performed in individual vessels, for example, within individual wells of a multi-well plate or other vessel.

Determining the identity of a genetic variation can also include or consist of reviewing a subject's medical history, where the medical history includes information regarding the identity, copy number, presence or absence of one or more alleles or SNPs in the subject, e.g., results of a genetic test.

Genetic variations can also be identified using any of a number of methods well known in the art. For example, genetic variations available in public databases, which can be searched using methods and custom algorithms or algorithms known in the art, can be used. A reference sequence can be from, for example, the human draft genome sequence, publicly available in various databases, or a sequence deposited in a database such as GenBank.

Another variation on the array-based approach can be to use the hybridization signal intensities that are obtained from the oligonucleotides employed on Affymetrix SNP arrays or in Illumina Bead Arrays. Here hybridization intensities are compared with average values that are derived from controls, such that deviations from these averages indicate a change in copy number. As well as providing information about copy number, SNP arrays have the added advantage of providing genotype information. For example, they can reveal loss of heterozygosity, which could provide supporting evidence for the presence of a deletion, or might indicate segmental uniparental disomy (which can recapitulate the effects of structural variation in some genomic regions—Prader-Willi and Angelman syndromes, for example).

Many of the basic procedures followed in microarray-based genome profiling are similar, if not identical, to those followed in expression profiling and SNP analysis, including the use of specialized microarray equipment and data-analysis tools. Since microarray-based expression profiling has been well established in the art, much can be learned from the technical advances made in this area. Examples of the use of microarrays in nucleic acid analysis that can be used are described in U.S. Pat. No. 6,300,063, U.S. Pat. No. 5,837,832, U.S. Pat. No. 6,969,589, U.S. Pat. No. 6,040,138, U.S. Pat. No. 6,858,412, U.S. application Ser. No. 08/529,115, U.S. application Ser. No. 10/272,384, U.S. application Ser. No. 10/045,575, U.S. application Ser. No. 10/264,571 and U.S. application Ser. No. 10/264,574. It should be noted that there are also distinct differences such as target and probe complexity, stability of DNA over RNA, the presence of repetitive DNA and the need to identify single copy number alterations in genome profiling.

The presence or absence of the disease or disorder in the subject can be determined with at least 50% confidence. For example, the presence or absence of the disease or disorder in the subject can be determined with at least 50%, 55%, 60%, 65%, 70%, 75%, 80%, 85%, 90%, 95%, 98%, 99%, or 100% confidence. The presence or absence of the disease or disorder in the subject can be determined with a from 50% to 100% confidence. For example, the presence or absence of the disease or disorder in the subject can be determined with a confidence of from about 60% to about 100%, 70% to about 100%, 80% to about 100%, 90% to about 100%, 50% to about 90%, 50% to about 80%, 50% to about 70%, 50% to about 60%, 60% to about 90%, 60% to about 80%, 60% to about 70%, 70% to about 90%, 70% to about 80%, or 80% to about 90%.

### Computer-Implemented Aspects

As understood by those of ordinary skill in the art, the methods and information described herein (genetic variation association with neurological disorders) can be implemented, in all or in part, as computer executable instructions on known computer readable media. For example, the methods described herein can be implemented in hardware. Alternatively, the method can be implemented in software stored in, for example, one or more memories or other computer readable medium and implemented on one or more processors. As is known, the processors can be associated with one or more controllers, calculation units and/or other units of a computer system, or implanted in firmware as desired. If implemented in software, the routines can be stored in any computer readable memory such as in RAM, ROM, flash memory, a magnetic disk, a laser disk, or other storage medium, as is also known. Likewise, this software can be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above can be implemented as various blocks, operations, tools, modules and techniques which, in turn, can be implemented in hardware, firmware, software, or any combination of hardware, firmware, and/or software. When implemented in hardware, some or all of the blocks, operations, techniques, etc. can be implemented in, for example, a custom integrated circuit (IC), an application specific integrated circuit (ASIC), a field programmable logic array (FPGA), a programmable logic array (PLA), etc.

Results from such genotyping can be stored in a data storage unit, such as a data carrier, including computer databases, data storage disks, or by other convenient data storage means. In certain embodiments, the computer database is an object database, a relational database or a post-relational database. Data can be retrieved from the data storage unit using any convenient data query method.

When implemented in software, the software can be stored in any known computer readable medium such as on a magnetic disk, an optical disk, or other storage medium, in a RAM or ROM or flash memory of a computer, processor, hard disk drive, optical disk drive, tape drive, etc. Likewise, the software can be delivered to a user or a computing system via any known delivery method including, for example, on a computer readable disk or other transportable computer storage mechanism.

The steps of the claimed methods can be operational with numerous other general purpose or special purpose computing system environments or configurations. Examples of well-known computing systems, environments, and/or configurations that can be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the methods and systems described herein can be described in the general context of computer-executable instructions, such as program modules, being executed by a computer. Generally, program modules include routines, programs, objects, components, and/or data structures that perform particular tasks or implement particular abstract data types. The methods and apparatus can also be practiced in distributed computing environments where tasks are performed by remote processing devices that are linked through a communications network. In both integrated and distributed computing environments, program modules can be located in both local and remote computer storage media including memory storage devices. Numerous alternative embodiments could be implemented, using either current technology or technology developed after the filing date of this application, which would still fall within the scope of the claims defining the disclosure.

The methods disclosed herein can be implemented in software, they can be implemented in hardware, firmware, etc., and can be implemented by any other processor. Thus, the elements described herein can be implemented in a standard multi-purpose CPU or on specifically designed hardware or firmware such as an application-specific integrated circuit (ASIC) or other hard-wired device as desired. When implemented in software, the software routine can be stored in any computer readable memory such as on a magnetic disk, a laser disk, or other storage medium, in a RAM or ROM of a computer or processor, in any database, etc. Likewise, this software can be delivered to a user or a screening system via any known or desired delivery method including, for example, on a computer readable disk or other transportable computer storage mechanism or over a communication channel, for example, a telephone line, the internet, or wireless communication. Modifications and variations can be made in the techniques and structures described and illustrated herein without departing from the spirit and scope of the present disclosure.

### Treatment and Therapy

The invention provides several methods of treating or effecting prophylaxis a neurological disease or disorder, for example MSA. In some cases, the invention provides several methods of treating MSA. In some cases, the invention provides several methods of treating LRRK2 related diseases, Lewy Body, or synucleinopathies related diseases in patients suffering from or at risk of such diseases. Patients amenable to treatment include individuals at risk of a disease disclosed herein but not showing symptoms, as well as patients presently showing symptoms or the early warning signs of synucleinopathies, for example, EEG slowing, neuropsychiatric manifestations (depression, dementia, hallucinations, anxiety, apathy, anhedonia), autonomic changes (orthostatic hypotension, bladder disturbances, constipation, fecal incontinence, sialorrhea, dysphagia, sexual dysfunction, changes in cerebral blood flow), sensory changes (olfactory, pain, color discrimination abnormal sensations), sleep disorders (REM sleep behavior disorder (RBD), restless legs syndrome/periodic extremity movements, hypersomnia, insomnia), resting tremor, muscular rigidity, bradykinesia, postural instability, and miscellaneous other signs and symptoms (fatigue, diplopia, blurred vision, seborrhea, weight loss/gain). Therefore, the present methods can be administered prophylactically to individuals who have a known genetic risk of a disclosed disease. Such individuals include those having relatives who have experienced this disease and those whose risk is determined by analysis of genetic or biochemical markers.

In asymptomatic or symptomatic patients, treatment can begin at any age (e.g., 5, 10, 20, 30, 40, 50, 60 or 70). Usually, however, it may not be necessary to begin treatment until a patient reaches 35, 40, 50, 60 or 70. Treatment can entail a single dose or multiple dosing over a period of time. In some cases, treatment can typically entail multiple dosages over a period of time. Treatment can be monitored by evaluating symptoms, assaying antibody, or activated T-cell or B-cell responses to a therapeutic agent over time. In some cases, a booster dosage can be administered. In some cases, if the response to an administered dose falls, a booster dosage can be indicated.

In prophylactic applications of a treatment described herein, a treatment, e.g., an antibody or a pharmaceutical composition, can be administered to a patient susceptible to, or otherwise at risk of a disease in a regime (dose, frequency and route of administration) effective to reduce the risk, lessen the severity, or delay the onset of at least one sign or symptom of the disease. In some prophylactic applications, the regime is effective to inhibit or delay accumulation of alpha synuclein and/or truncated fragments in the brain, and/or inhibit or delay its toxic effects and/or inhibit/or delay development of behavioral deficits. In therapeutic applications, a treatment is administered to a patient suspected of, or already suffering from, a disease described herein in a regime (dose, frequency, and route of administration) effective to ameliorate or at least inhibit further deterioration of at least one sign or symptom of the disease. In some therapeutic applications, the regime is effective to reduce or at least inhibit further increase of levels of alpha synuclein, truncated fragments, associated toxicities, behavioral deficits, or symptoms.

A regime can be considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase or phase III trial).

An effective dose can vary depending on many different factors, including means of administration, target site, physiological state of the patient including whether the patient is human or an animal, other medications administered, and whether treatment is prophylactic or therapeutic.

An exemplary dosage range for antibodies can be from about 0.01 to 5 mg/kg, and more usually 0.1 to 3 mg/kg or 0.15-2 mg/kg or 0.15-1.5 mg/kg or more, of patient body weight. A treatment can administer such doses daily, on alternative days, weekly, fortnightly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. In some cases, a subject can be given a treatment, and there after evaluated for continued treatment.

A therapeutically effective amount of a treatment can be dependent on the weight of a subject. In some cases, the therapeutically effective amount of a treatment is at least about 1μg of a treatment per kg of the subject, for example at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg of a treatment per kg of the subject. In some cases, the therapeutically effective amount of a treatment is at least about 1 mg of a treatment per kg of the subject, for example at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg of a treatment per kg of the subject. In some cases, the therapeutically effective amount of a treatment is less than about 1000 μg of a treatment per kg of the subject, for example less than about 1, 2, 3, 4, 5, 6, 7, 8, 9, 10, 20, 30, 40, 50, 60, 70, 80, 90, 100, 200, 300, 400, 500, 600, 700, 800, 900, or 1000 μg of a treatment per kg of the subject. In some cases, the therapeutically effective amount of a treatment is less than about 1000 mg of a treatment per kg of the subject, for example less than about 1, 2, 3, 4, 5, 6, 7, 8, 9, 10, 20, 30, 40, 50, 60, 70, 80, 90, 100, 200, 300, 400, 500, 600, 700, 800, 900, or 1000 mg of a treatment per kg of the subject. In some cases, the therapeutically effective amount of a treatment ranges from about 1 μg to about 1000 μg of a treatment per kg of the subject, for example from about 1 to about 700, 1 to about 500, 1 to about 300, 1 to about 100, 1 to about 50, 1 to about 10, 10 to about 700, 10 to about 500, 10 to about 300, 10 to about 100, 10 to about 80, 10 to about 60, 10 to about 40, 10 to about 20, 50 to about 700, 50 to about 500, 50 to about 300, 50 to about 100, 100 to about 700, 100 to about 500, 100 to about 300, 300 to about 700, 300 to about 500, or 500 to about 700 μg of a treatment per kg of the subject. In some cases, the therapeutically effective amount of a treatment ranges from about 1 μg to about 10 μg of a treatment per kg of the subject. In some cases, the therapeutically effective amount of a treatment ranges from about 10 μg to about 100 μg of a treatment per kg of the subject. In some cases, the therapeutically effective amount of a treatment ranges from about 100 μg to about 500 μg of a treatment per kg of the subject. In some cases, the therapeutically effective amount of a treatment ranges from about 1μg to about 1000 mg of a treatment per kg of the subject, for example from about 1 to about 700, 1 to about 500, 1 to about 300, 1 to about 100, 1 to about 50, 1 to about 10, 10 to about 700, 10 to about 500, 10 to about 300, 10 to about 100, 10 to about 80, 10 to about 60, 10 to about 40, 10 to about 20, 50 to about 700, 50 to about 500, 50 to about 300, 50 to about 100, 100 to about 700, 100 to about 500, 100 to about 300, 300 to about 700, 300 to about 500, or 500 to about 700 mg of a treatment per kg of the subject. In some cases, the therapeutically effective amount of a treatment ranges from about 16 mg to about 24 mg of a treatment per kg of the subject. In some cases, the therapeutically effective amount of a treatment ranges from about 30 mg to about 100 mg of a treatment per kg of the subject. In some cases, the therapeutically effective amount of a treatment ranges from about 50 mg to about 140 mg of a treatment per kg of the subject. In some cases, the therapeutically effective amount of a treatment ranges from about 115 mg to about 125 mg of a treatment per kg of the subject. The therapeutically effective amount of a treatment can also be the daily dosage of a treatment for the subject.

A treatment described herein can be e.g., antibodies, can be administered. Routes of administration can include topical, intravenous, oral, subcutaneous, intra-arterial, intracranial, intrathecal, intraperitoneal, intranasal, or intramuscular. Some routes for administration can be intravenous or subcutaneous. A treatment, for example an antibody can be injected in the arm or leg muscles. In some methods, a treatment can be injected directly into a particular tissue where deposits have accumulated, for example intracranial injection.

Pharmaceutical compositions can be sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, treatments can be formulated in aqueous solutions, preferably in physiologically compatible buffers such as Hanks' solution, Ringer's solution, or physiological saline or acetate buffer (to reduce discomfort at the site of injection). The solution can contain formulatory agents such as suspending, stabilizing, and/or dispersing agents. Alternatively treatments can be in lyophilized form for constitution with a suitable vehicle, e.g., sterile pyrogen-free water, before use.

The present regimes can be administered in combination with another agent effective in treatment or prophylaxis of the disease being treated. For example, immunotherapy against alpha synuclein WO/2008/103472, Levodopa, dopamine agonists, COMT inhibitors, MAO-B inhibitors, Amantadine, or anticholinergic agents can be used in combination with the present regimes.

A treatment described herein can increase cognitive function of a subject. In some cases, a treatment described herein can increase cognitive function of a subject afflicted with a disease disclosed herein, for example MSA. Cognitive function can be measured by methods known in the art. In some cases, cognitive function can be measured using a maze in which subjects use spatial information, fear conditioning, or active avoidance.

Cognitive function can be measured by one or more of several standardized tests. Examples of a test or assay for cognitive function were described (Ruoppila and Suutama, Scand. J. Soc. Med. Suppl. 53,44-65, 1997) and include standardized psychometric tests (e. g. Wechsler Memory Scale, the Wechsler Adult Intelligence Scale, Raven's Standard Progressive Matrices, Schaie-Thurstone Adult Mental Abilities Test), neuropsychological tests (e. g. Luria-Nebraska), metacognitive self-evaluations (e. g. Metamemory Questionnaire), visual-spatial screening tests (e.g., Poppelreuter's Figures, Clock Recognition, Honeycomb Drawing and Cancellation), cognitive screening tests (e. g. Folstein's Mini Mental State Test) and reaction time tests. Other standard tests for cognitive performance include the Alzheimer's Disease Assessment Scale-cognitive subscale (ADAS-cog); the clinical global impression of change scale (CIBIC-plus scale); the Alzheimer's Disease Cooperative Study Activities of Daily Living Scale (ADCS-ADL); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG); Stroop Test; Trail Making; Wechsler Digit Span; and the CogState computerized cognitive test. In addition, cognitive function may be measured using imaging techniques such as Positron Emission Tomography (PET), functional magnetic resonance imaging (fMRI), Single Photon Emission Computed Tomography (SPECT), or any other imaging technique that allows one to measure brain function.

### Multiple System Atrophy Therapeutics

There is currently no cure for MSA, but medications, surgery, and multidisciplinary management can provide relief from the symptoms. In some cases, the treatments disclosed herein can be used individually or in combination. In some cases, a family of drugs useful for treating motor symptoms can include levodopa (usually combined with a dopa decarboxylase inhibitor or COMT inhibitor), dopamine agonists and MAO-B inhibitors. In some cases, when medications are not enough to control symptoms, surgery and deep brain stimulation can be of use. In the final stages of the disease, palliative care can be provided to enhance quality of life. Carbidopa and benserazide are peripheral dopa decarboxylase inhibitors, which can help to prevent the metabolism of L-DOPA before it reaches dopaminergic neurons, therefore reducing side effects and increasing bioavailability. Existing preparations are carbidopa/levodopa (co-careldopa) and benserazide/levodopa (co-beneldopa). Tolcapone inhibits the COMT enzyme. In some instances, tolcapone can be used to complement levodopa. A similarly effective drug, entacapone, has not been shown to cause significant alterations of liver function. Licensed preparations of entacapone can contain entacapone alone or in combination with carbidopa and levodopa. Dopamine agonists can include but is not limited to bromocriptine, pergolide, pramipexole, ropinirole, piribedil, cabergoline, apomorphine, and lisuride.

Apomorphine, a non-orally administered dopamine agonist, may be used. MAO-B inhibitors (selegiline and rasagiline) can increase the level of dopamine in the basal ganglia by blocking its metabolism. Other drugs such as amantadine and anticholinergics can be useful as treatment of motor symptoms. Other treatments are available to treat additional symptoms of MSA. Examples are the use of clozapine for psychosis, cholinesterase inhibitors for dementia, and modafinil for daytime sleepiness. In some cases, non-steroidal anti-inflammatory drugs can be used to reduce and/or delay the development of MSA.

Symptoms of MSA can be treated with surgery. Surgery can include lesional and deep brain stimulation (DBS). Other surgical therapies can involve the formation of lesions in specific subcortical areas (a technique known as pallidotomy in the case of the lesion being produced in the globus pallidus).

Speech or mobility symptoms can be improved with rehabilitation. Regular physical exercise with or without physiotherapy can be beneficial to maintain and improve mobility, flexibility, strength, gait speed, and quality of life. Other effective techniques to promote relaxation can include slow rotational movements of the extremities and trunk, rhythmic initiation, diaphragmatic breathing, and meditation techniques. Physiotherapists have a variety of strategies to improve functional mobility and safety. In some cases, Lee Silverman voice treatment (LSVT) can be used to treat a subject afflicted with MSA. Occupational therapy (OT) aims to promote health and quality of life by helping people with the disease to participate in as many of their daily living activities as possible. OT can be used to treat a subject afflicted with MSA. OT can improve motor skills and quality of life for the duration of the therapy.

A person skilled in the art will appreciate and understand that the genetic variants described herein in general may not, by themselves, provide an absolute identification of individuals who can develop a neurological disorder or related conditions. The variants described herein can indicate increased and/or decreased likelihood that individuals carrying the at-risk or protective variants of the disclosure can develop symptoms associated with a neurological disorder. This information can be used to, for example, initiate preventive measures at an early stage, perform regular physical and/or mental exams to monitor the progress and/or appearance of symptoms, or to schedule exams at a regular interval to identify early symptoms, so as to be able to apply treatment at an early stage. Screening criteria can comprise a number of symptoms to be present over a period of time; therefore, it is important to be able to establish additional risk factors that can aid in the screening, or facilitate the screening through in-depth phenotyping and/or more frequent examination, or both. For example, individuals with early symptoms that typically are not individually associated with a clinical screening of a neurological disorder and carry an at-risk genetic variation can benefit from early therapeutic treatment, or other preventive measure, or more rigorous supervision or more frequent examination. Likewise, individuals that have a family history of the disease, or are carriers of other risk factors associated with a neurological disorder can, in the context of additionally carrying at least one at-risk genetic variation, benefit from early therapy or other treatment.

Early symptoms of behavioral disorders such as a neurological disorder and related conditions may not be sufficient to fulfill standardized screening criteria. To fulfill those, a certain pattern of symptoms and behavioral disturbance may need to manifest itself over a period of time. Sometimes, certain physical characteristics can also be present. This makes at-risk genetic variants valuable in a screening setting, in particular high-risk variants. Determination of the presence of such variants warrants increased monitoring of the individual in question. Appearance of symptoms combined with the presence of such variants facilitates early screening, which makes early treatment possible. Genetic testing can thus be used to aid in the screening of disease in its early stages, before all criteria for formal screening criteria are all fulfilled. It is well established that early treatment is extremely important for neurological disorders and related disorders, which lends further support to the value of genetic testing for early diagnosis, prognosis, or theranosis of these disorders.

Variant gene expression in a subject can be assessed by expression of a variant-containing nucleic acid sequence or by altered expression of a normal/wild-type nucleic acid sequence due to variants affecting the level or pattern of expression of the normal transcripts, for example, variants in the regulatory or control region of the gene. Assays for gene expression include direct nucleic acid assays (mRNA), assays for expressed polypeptide levels, or assays of collateral compounds involved in a pathway, for example, a signal pathway. Furthermore, the expression of genes that are up-or down-regulated in response to the signal pathway can also be assayed. One embodiment includes operably linking a reporter gene, such as luciferase, to the regulatory region of one or more gene of interest.

Therapeutic agents can comprise one or more of, for example, small non-polypeptide and non-nucleic acids, polypeptides, peptides, polypeptide fragments, nucleic acids (RNA, DNA, RNAJ, PNA (peptide nucleic acids), or their derivatives or mimetics which can modulate the function and/or levels of a target genes or their protein products. Treatment of MSA can comprise treatment of one of the genes, or protein products derived thereof, such as mRNA or a polypeptide, with one or more of the therapeutics disclosed herein. Treatment of MSA can comprise treatment of 2, 3, 4, 5, 6, 7, 8, 9, 10, or more of the genes, or protein products derived there from, with 2, 3, 4, 5, 6, 7, 8, 9, 10, or more of the therapeutics disclosed herein.

### LRRK2 Inhibitors

A LRRK2 inhibitor can be administered to treat neurodegenerative diseases. A LRRK2 inhibitor can be administered to treat MSA and related symptoms. A LRRK2 inhibitor can be a staurosporine derivative, a naleimide derivative, an indolinone dervivative, a quinoline derivative, an anthracene derivative, a phenanthrene derivative, or a pyrimidine derivative. A LRRK2 inhibitor can be at least one of PD-98059, U-0126, SB-203580, H-7, H-9, Staurosporine, AG-494, AG-825, Lavendustin A, RG-14620, Tyrphostin 23, Tyrphostin 25, Tyrphostin 46, Tyrphostin 47, Tyrphostin 51, Tyrphostin 1, Tyrphostin AG1288, Tyrphostin AG1478, Tyrphostin AG1295, Tyrphostin 9, hydroxy-2-naphthalene ethylphosphoric acid, Damnacanthal, piceatannol, PP1, AG-490, AG-126, AG-370, AG-879, LY294002, wortmannin, GP109203X, hypericin, Ro31-8220, Sphingosine, H-89, H-8, HA-1004, HA-1077, 2-hydroxy-5-(2,5-dihydroxybenzyl-amino) benzoic acid, KN-62, KN-93, ML-7, ML-9, 2-aminopurine, N9-isopropyl olomoucine, Olomoucine, iso-olomoucine, roscovitine, 5-iodo-tubercidin, LFM-A13, SB-202190, PP2, ZM336372, SU4312, AG-1296, GW5074, palmitoyl-DL-carnitine Cl, rottlerin, genistein, daidzein, erbstatin analog, quercetin dihydrate, SU1498, ZM449829, BAY11-7082, 5,6-dichloro-1-beta-D-ribofutanosyl-benzimidiazole, 2,2′,3,3′,4,4′-hexahydroxy-1,1′-biphenyl-6,6′-dimenthanol dimethyl ester, SP600126, Indirubin, Indirubin-3-monooxine, cantharidic acid, cantharidin, endothall, benzyl-phosphoric acid, L-p-bromo-tetraamisole oxalate, RK-682, RWJ-60475, levarmisole HCl, tetramisole HCl, cypermethrin, deltamethrin, fenvaierate, Tyrphostin 8, Cinngel, LDN-22684, LDN-73794, Y-27632, Compound 4, CZC-54252, CZC-25146, Go6976, K252b, LRRK2-in-1, H-1152, sunitinib, K252a, derivatives and analogs thereof. A LRRK2 inhibitor can be administered in combination with any one or more treatment/therapy disclosed herein.

TABLE 3

| LRRK2 | | |
|---|---|---|
| Inhibitor | Compound | |
| Number | Name | Chemical Structure |
| 1 | PD-98059 | 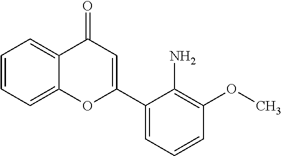 [SMILES: COc1cccc(-c2cc(=O)c3ccccc3o2)c1N] |
| 2 | U-0126 | 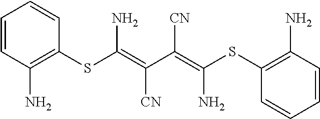 [SMILES: N#CC(=C(/N)Sc1ccccc1N)/C(C#N)=C(\N)Sc1ccccc1N] |
| 3 | SB-203580 | 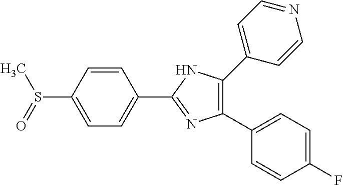 [SMILES: CS(=O)c1ccc(-c2nc(-c3ccc(F)cc3)c(-c3ccncc3)[nH]2)cc1] |
| 4 | H-7 | 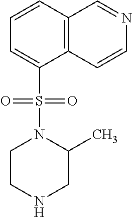 [SMILES: CC1CNCCN1S(=O)(=O)c1cccc2cnccc12] |
| 5 | H-9 | 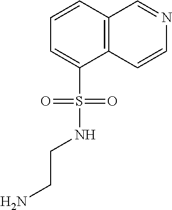 [SMILES: NCCNS(=O)(=O)c1cccc2cnccc12] |
| 6 | Staurosporine | 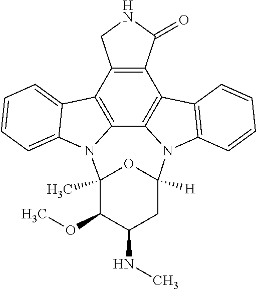 [SMILES: CN[C@@H]1C[C@H]2O[C@@](C)([C@@H]1OC)n1c3ccccc3c3c4c(c5c6ccccc6n2c5c31)C(=O)NC4] |
| 7 | AG-494 | 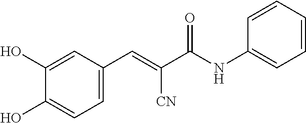 [SMILES: N#C/C(=C\c1ccc(O)c(O)c1)C(=O)Nc1ccccc1] |
| 8 | AG-825 | 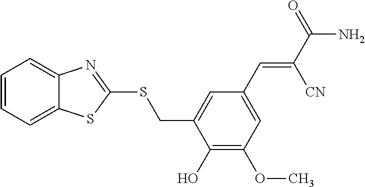 [SMILES: COc1cc(/C=C(\C#N)C(N)=O)cc(CSc2nc3ccccc3s2)c1O] |
| 9 | Lavendustin A | 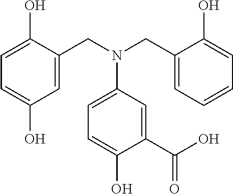 [SMILES: O=C(O)c1cc(N(Cc2ccccc2O)Cc2cc(O)ccc2O)ccc1O] |
| 10 | RG-14620 | 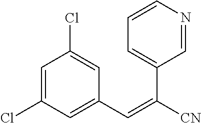 [SMILES: N#C/C(=C/c1cc(Cl)cc(Cl)c1)c1cccnc1] |
| 11 | Tyrphostin 23 | 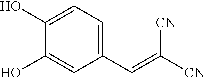 [SMILES: N#CC(C#N)=Cc1ccc(O)c(O)c1] |
| 12 | Tyrphostin 25 | 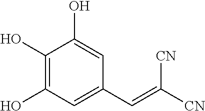 [SMILES: N#CC(C#N)=Cc1cc(O)c(O)c(O)c1] |
| 13 | Tyrphostin 46 | 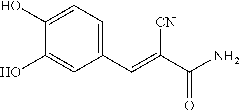 [SMILES: N#C/C(=C\c1ccc(O)c(O)c1)C(N)=O] |
| 14 | Tyrphostin 47 | 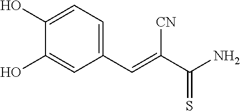 [SMILES: N#C/C(=C\c1ccc(O)c(O)c1)C(N)=S] |
| 15 | Tyrphostin 51 | 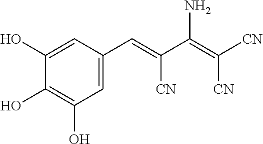 [SMILES: N#CC(C#N)=C(N)/C(C#N)=C/c1cc(O)c(O)c(O)c1] |
| 16 | Tyrphostin 1 | 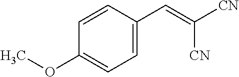 |
| 17 | Tyrphostin AG1288 | 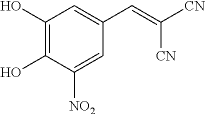 [SMILES: N#CC(C#N)=Cc1cc(O)c(O)c([N+](=O)[O-])c1] |
| 18 | Tyrphostin AG1478 | 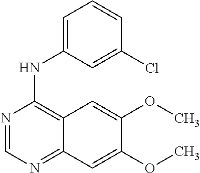 [SMILES: COc1cc2ncnc(Nc3cccc(Cl)c3)c2cc1OC] |
| 19 | Tyrphostin AG1295 | 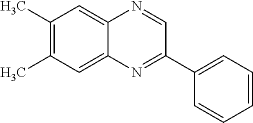 [SMILES: Cc1cc2ncc(-c3ccccc3)nc2cc1C] |
| 20 | Tyrphostin 9 | 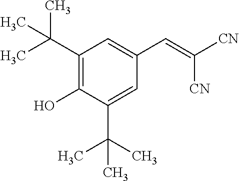 [SMILES: CC(C)(C)c1cc(C=C(C#N)C#N)cc(C(C)(C)C)c1O] |
| 21 | Hydroxy-2- naphthalene ethylphosphoric acid | 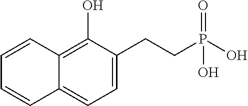 [SMILES: O=P(O)(O)CCc1ccc2ccccc2c1O] |
| 22 | Damnacanthal | 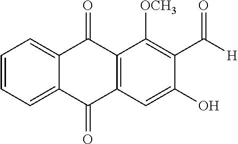 [SMILES: COc1c(C=O)c(O)cc2c1C(=O)c1ccccc1C2=O] |
| 23 | Piceatannol | 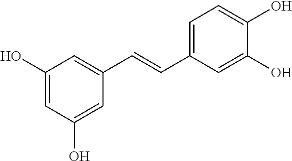 [SMILES: Oc1cc(O)cc(/C=C/c2ccc(O)c(O)c2)c1] |
| 24 | PP1 | 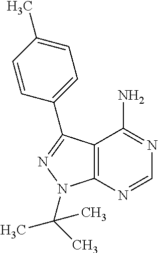 [SMILES: Cc1ccc(-c2nn(C(C)(C)C)c3ncnc(N)c23)cc1] |
| 25 | AG-490 | 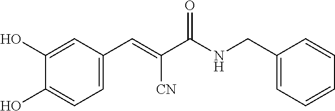 [SMILES: N#C/C(=C\c1ccc(O)c(O)c1)C(=O)NCc1ccccc1] |
| 26 | AG-126 | 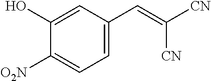 [SMILES: N#CC(C#N)=Cc1ccc([N+](=O)[O-])c(O)c1] |
| 27 | AG-370 | 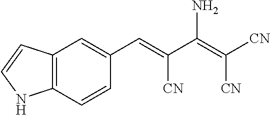 [SMILES: N#CC(C#N)=C(N)/C(C#N)=C/c1ccc2[nH]ccc2c1] |
| 28 | AG-879 | 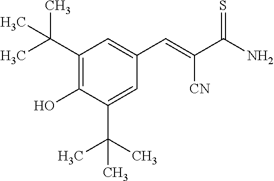 [SMILES: CC(C)(C)c1cc(/C=C(\C#N)C(N)=S)cc(C(C)(C)C)c1O] |
| 29 | LY294002 | 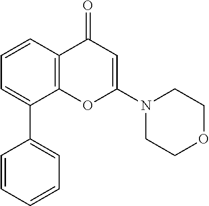 [SMILES: O=c1cc(N2CCOCC2)oc2c(-c3ccccc3)cccc12] |
| 30 | Wortmannin | 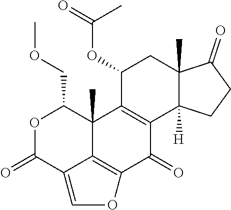 [SMILES: COC[C@H]1OC(=O)c2coc3c2[C@@]1(C)C1=C(C3=O)[C@@H]2CCC(=O)[C@@]2(C)C[C@H]1OC(C)=O] |
| 31 | GP109203X | 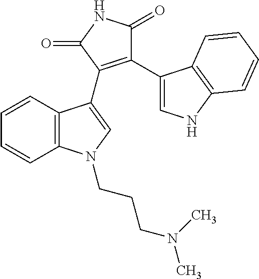 [SMILES: CN(C)CCCn1cc(C2=C(c3c[nH]c4ccccc34)C(=O)NC2=O)c2ccccc21] |
| 32 | Hypercin | 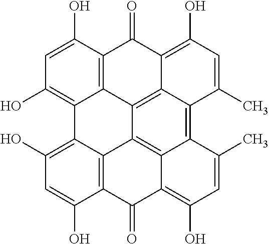 [SMILES: Cc1cc(O)c2c(=O)c3c(O)cc(O)c4c5c(O)cc(O)c6c(=O)c7c(O)cc(C)c8c1c2c(c34)c(c78)c65] |
| 33 | Ro31-8220 | 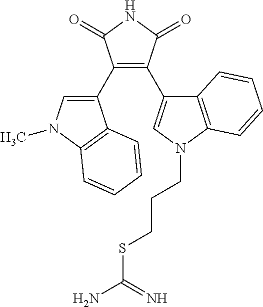 [SMILES: Cn1cc(C2=C(c3cn(CCCSC(=N)N)c4ccccc34)C(=O)NC2=O)c2ccccc21] |
| 34 | Sphingosine | 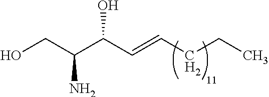 |
| 35 | H-89 | 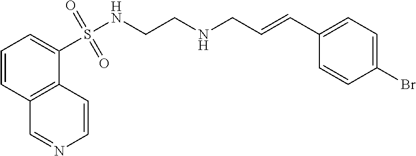 [SMILES: O=S(=O)(NCCNC/C=C/c1ccc(Br)cc1)c1cccc2cnccc12] |
| 36 | H-8 | 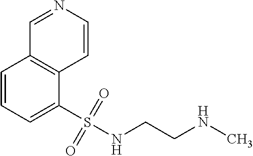 [SMILES: CNCCNS(=O)(=O)c1cccc2cnccc12] |
| 37 | HA-1004 | 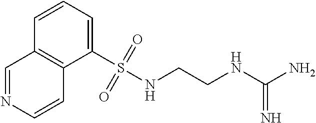 [SMILES: N=C(N)NCCNS(=O)(=O)c1cccc2cnccc12] |
| 38 | HA-1077 | 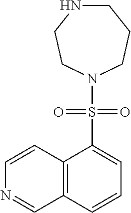 [SMILES: O=S(=O)(c1cccc2cnccc12)N1CCCNCC1] |
| 39 | 2-hydroxy-5- (2,5- dihydroxybenzyl- amino) benzoic acid | 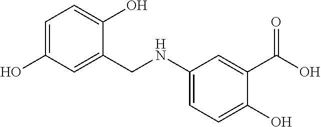 [SMILES: O=C(O)c1cc(NCc2cc(O)ccc2O)ccc1O] |
| 40 | KN-62 | 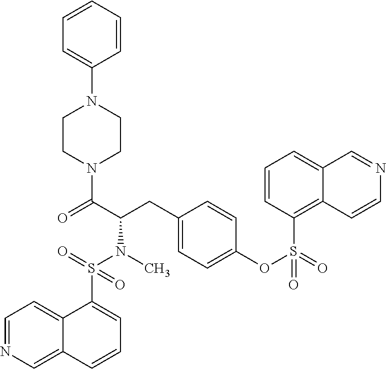 [SMILES: CN([C@@H](Cc1ccc(OS(=O)(=O)c2cccc3cnccc23)cc1)C(=O)N1CCN(c2ccccc2)CC1)S(=O)(=O)c1cccc2cnccc12] |
| 41 | KN-93 | 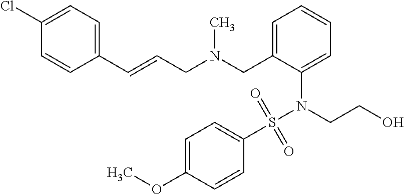 [SMILES: COc1ccc(S(=O)(=O)N(CCO)c2ccccc2CN(C)C/C=C/c2ccc(Cl)cc2)cc1] |
| 42 | ML-7 | 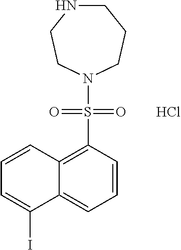 [SMILES: O=S(=O)(c1cccc2c(I)cccc12)N1CCCNCC1 ; Cl] |
| 43 | ML-9 | 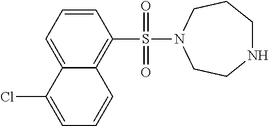 [SMILES: O=S(=O)(c1cccc2c(Cl)cccc12)N1CCCNCC1] |
| 44 | 2-aminopurine | 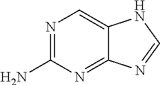 |
| 45 | N9-isopropyl olomoucine | 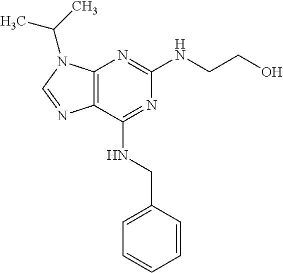 [SMILES: CC(C)n1cnc2c(NCc3ccccc3)nc(NCCO)nc21] |
| 46 | Olomoucine | 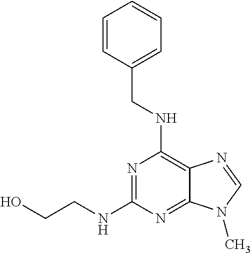 [SMILES: Cn1cnc2c(NCc3ccccc3)nc(NCCO)nc21] |
| 47 | Iso-olomoucine | 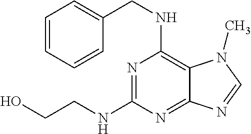 [SMILES: Cn1cnc2nc(NCCO)nc(NCc3ccccc3)c21] |
| 48 | Roscovitine | 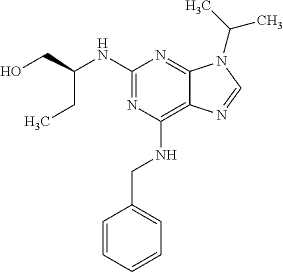 [SMILES: CC[C@@H](CO)Nc1nc(NCc2ccccc2)c2ncn(C(C)C)c2n1] |
| 49 | 5-iodo- tubercidin | 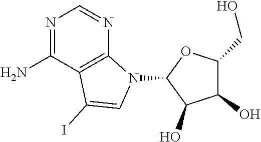 [SMILES: Nc1ncnc2c1c(I)cn2[C@@H]1O[C@H](CO)[C@@H](O)[C@H]1O] |
| 50 | LFM-A13 | 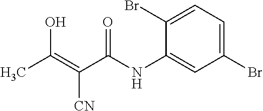 [SMILES: C/C(O)=C(\C#N)C(=O)Nc1cc(Br)ccc1Br] |
| 51 | SB-202190 | 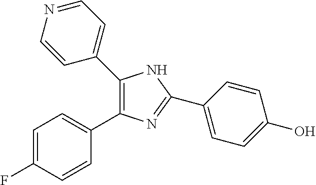 [SMILES: Oc1ccc(-c2nc(-c3ccc(F)cc3)c(-c3ccncc3)[nH]2)cc1] |
| 52 | PP2 | 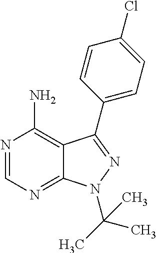 [SMILES: CC(C)(C)n1nc(-c2ccc(Cl)cc2)c2c(N)ncnc21] |
| 53 | ZM336372 | 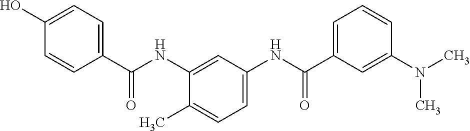 [SMILES: Cc1ccc(NC(=O)c2cccc(N(C)C)c2)cc1NC(=O)c1ccc(O)cc1] |
| 54 | SU4312 | 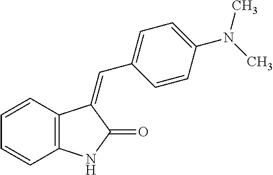 [SMILES: CN(C)c1ccc(/C=C2\C(=O)Nc3ccccc32)cc1] |
| 55 | AG-1296 | 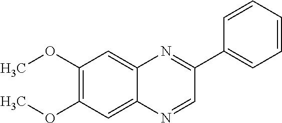 [SMILES: COc1cc2ncc(-c3ccccc3)nc2cc1OC] |
| 56 | GW5074 | 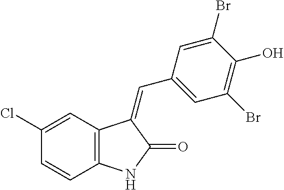 [SMILES: O=C1Nc2ccc(Cl)cc2/C1=C/c1cc(Br)c(O)c(Br)c1] |
| 57 | Palmitoyl-DL- carnitine Cl | 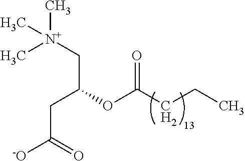 |
| 58 | Rottlerin | 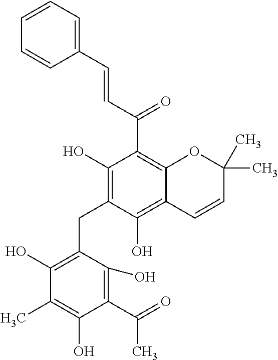 [SMILES: CC(=O)c1c(O)c(C)c(O)c(Cc2c(O)c3c(c(C(=O)/C=C/c4ccccc4)c2O)OC(C)(C)C=C3)c1O] |
| 59 | Genistein | 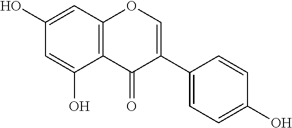 [SMILES: O=c1c(-c2ccc(O)cc2)coc2cc(O)cc(O)c12] |
| 60 | Daidzein | 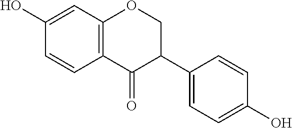 [SMILES: O=C1c2ccc(O)cc2OCC1c1ccc(O)cc1] |
| 61 | Erbstatin analog | 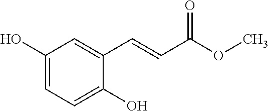 |
| 62 | Quercetin dihydrate | 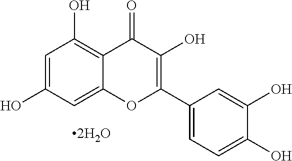 [SMILES: O=c1c(O)c(-c2ccc(O)c(O)c2)oc2cc(O)cc(O)c12 ; "·2H2O"] |
| 63 | SU1498 | 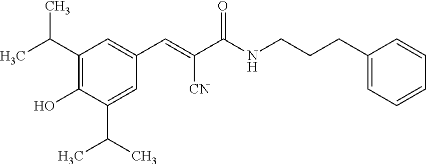 [SMILES: CC(C)c1cc(/C=C(\C#N)C(=O)NCCCc2ccccc2)cc(C(C)C)c1O] |
| 64 | ZM449829 | 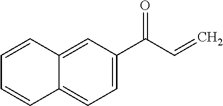 |
| 65 | BAY11-7082 | 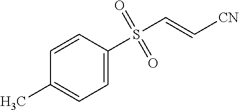 |
| 66 | 5,6-dichloro-1- beta-D- ribofutanosyl- benzimidiazole | 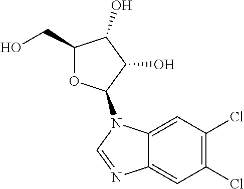 [SMILES: OC[C@@H]1O[C@H](n2cnc3cc(Cl)c(Cl)cc32)[C@@H](O)[C@H]1O] |
| 67 | 2,2′,3,3′,4,4′- hexahydroxy- 1,1′-biphenyl- 6,6′- dimenthanol dimethyl ester | 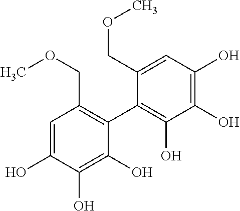 [SMILES: COCc1cc(O)c(O)c(O)c1-c1c(COC)cc(O)c(O)c1O] |
| 68 | SP600125 | 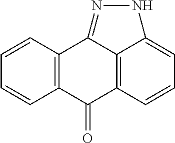 [SMILES: O=C1c2ccccc2-c2n[nH]c3cccc1c23] |
| 69 | Indirubin | 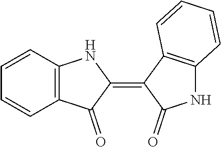 [SMILES: O=C1Nc2ccccc2/C1=C1\Nc2ccccc2C1=O] |
| 70 | Indirubin-3- monooxine | 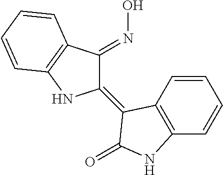 [SMILES: O=C1Nc2ccccc2/C1=C1/Nc2ccccc2/C1=N\O] |
| 71 | Cantharidic acid | 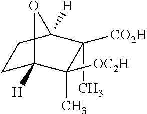 |
| 72 | Cantharidin | 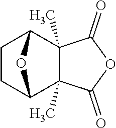 |
| 73 | Endothall | 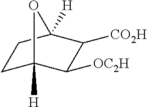 |
| 74 | Benzyl- phosphoric acid | 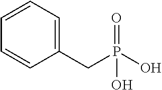 [SMILES: O=P(O)(O)Cc1ccccc1] |
| 75 | L-p-bromo- tetraamisole oxalate | 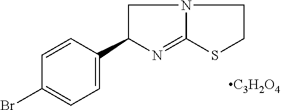 |
| 76 | RK-682 | 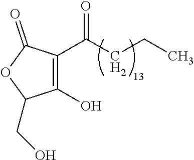 |
| 77 | RWJ-60475 | 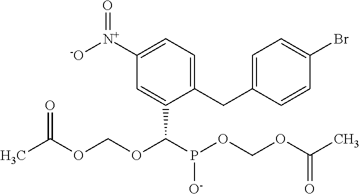 |
| 78 | Levarmisole HCl | 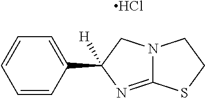 [SMILES: Cl ; c1ccc([C@H]2CN3CCSC3=N2)cc1] |
| 79 | Tetramisole HCl | 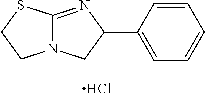 [SMILES: c1ccc(C2CN3CCSC3=N2)cc1 ; Cl] |
| 80 | Cypermethrin | 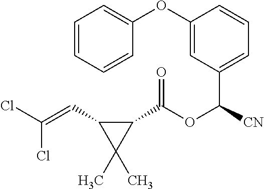 [SMILES: CC1(C)[C@H](C=C(Cl)Cl)[C@@H]1C(=O)O[C@H](C#N)c1cccc(Oc2ccccc2)c1] |
| 81 | Deltamethrin | 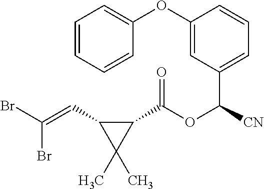 [SMILES: CC1(C)[C@H](C=C(Br)Br)[C@@H]1C(=O)O[C@H](C#N)c1cccc(Oc2ccccc2)c1] |
| 82 | Fenvaierate | 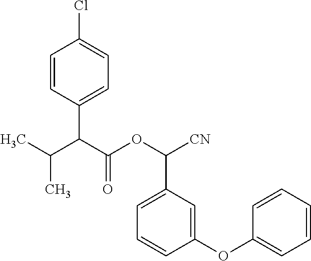 [SMILES: CC(C)C(C(=O)OC(C#N)c1cccc(Oc2ccccc2)c1)c1ccc(Cl)cc1] |
| 83 | Tyrphostin 8 | 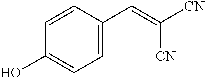 [SMILES: N#CC(C#N)=Cc1ccc(O)cc1] |
| 84 | Cinngel | 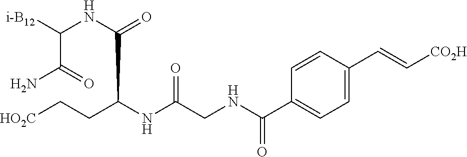 |
| 85 | LDN-22684 | 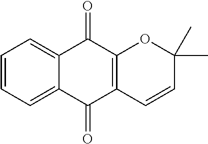 [SMILES: CC1(C)C=CC2=C(O1)C(=O)c1ccccc1C2=O] |
| 86 | LDN-73794 | 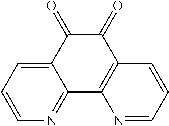 [SMILES: O=C1C(=O)c2cccnc2-c2ncccc21] |
| 87 | Y-27632 | 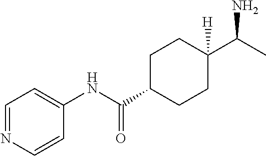 [SMILES: C[C@H](N)[C@H]1CC[C@H](C(=O)Nc2ccncc2)CC1] |
| 88 | Compound 4 | 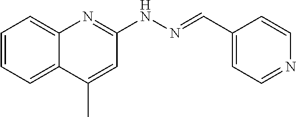 [SMILES: Cc1cc(N/N=C/c2ccncc2)nc2ccccc12] |
| 89 | CZC-54252 | 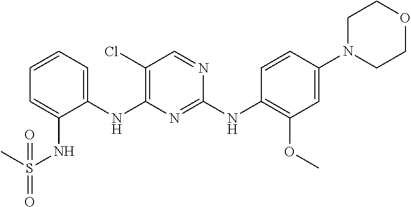 [SMILES: COc1cc(N2CCOCC2)ccc1Nc1ncc(Cl)c(Nc2ccccc2NS(C)(=O)=O)n1] |
| 90 | CZC-25146 | 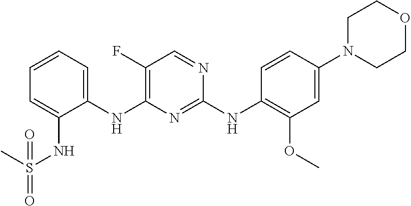 [SMILES: COc1cc(N2CCOCC2)ccc1Nc1ncc(F)c(Nc2ccccc2NS(C)(=O)=O)n1] |
| 91 | Go6976 | 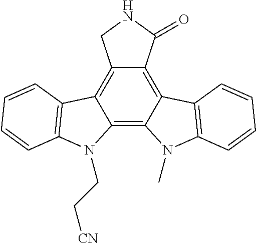 [SMILES: Cn1c2ccccc2c2c3c(c4c5ccccc5n(CCC#N)c4c21)CNC3=O] |
| 92 | K252b | 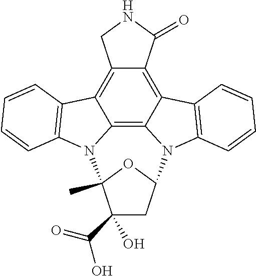 [SMILES: C[C@@]12O[C@@H](C[C@@]1(O)C(=O)O)n1c3ccccc3c3c4c(c5c6ccccc6n2c5c31)CNC4=O] |
| 93 | LRRK2-in-1 | 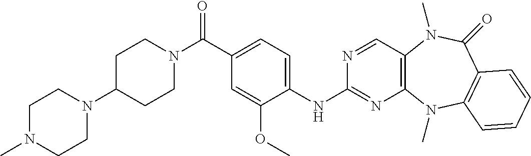 [SMILES: COc1cc(C(=O)N2CCC(N3CCN(C)CC3)CC2)ccc1Nc1ncc2c(n1)N(C)c1ccccc1C(=O)N2C] |
| 94 | H-1152 | 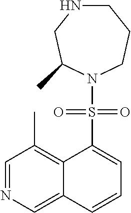 [SMILES: Cc1cncc2cccc(S(=O)(=O)N3CCCNC[C@@H]3C)c12] |
| 95 | Sunitinib | 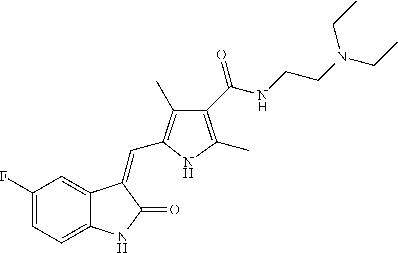 [SMILES: CCN(CC)CCNC(=O)c1c(C)[nH]c(/C=C2\C(=O)Nc3ccc(F)cc32)c1C] |
| 96 | K252a | 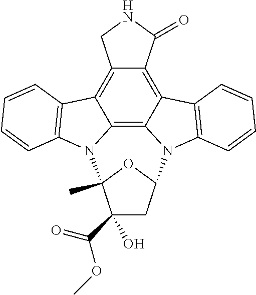 [SMILES: COC(=O)[C@]1(O)C[C@@H]2O[C@@]1(C)n1c3ccccc3c3c4c(c5c6ccccc6n2c5c31)C(=O)NC4] |

Protein kinases

Multiple small molecule kinase inhibitors have been approved by USA FDA and are available in the market, including imatinib (Gleevec), sorafenib (Nexavar), sunitinib (Sutent), rapamycin (Sirolimus) to name a few. Potential druggable kinase-related signaling pathways include protein kinase Cd, the MLK-cjun N-terminal kinase (JNK) signaling cascade, and AKT/protein kinase B (PKB) signaling cascade, all of which are kinases implicated in programmed cell death. CEP1347, an MLK inhibitor has been shown to have neuroprotective effects in a variety of neurodegenerative models. One or more protein kinase inhibitors disclosed herein can be used as a therapy to treat a neurological disease, for example MSA.

### Prodrugs

Prodrugs include compounds wherein an amino acid residue, or a polypeptide chain of two or more (e.g., two, three, or four) amino acid residues that are covalently joined through peptide bonds to free amino, hydroxy, or carboxylic acid groups of the parent compounds. Accordingly, some aspects of the invention provide a method for treating a neurodegenerative disease by administering a histone deacetylase inhibitor, or a derivative thereof, a prodrug thereof, or a salt thereof. Whether a particular compound is an HDAC inhibitor can be readily determined, for example, by in vitro experimentation. Such experimental procedures are well known to one skilled in the art. Moreover, many HDAC inhibitors are well known. Exemplary HDAC inhibitors include, but are not limited to, TSA; DP AH; Tubastatin A; MGCD; hydroxamic acids (or hydroxamates), such as trichostatin A, vorinostat (SAHA), belinostat, LAQ824, and panobinostat; cyclic tetrapeptides (such as trapoxin B); the depsipeptides; benzamides such as entinostat, CI994, and mocetinostat; electrophilic ketones; and the aliphatic acid compounds such as phenylbutyrate and valproic acid.

### RNA Therapeutics

The nucleic acids and/or variants of the disclosure, or nucleic acids comprising their complementary sequence, can be used as antisense constructs to control gene expression in cells, tissues, or organs. The methodology associated with antisense techniques is well known to the skilled artisan, and is described and reviewed in Antisense Drug Technology: Principles, Strategies, and Applications, Crooke, Marcel Dekker Inc., New York (2001) In general, antisense nucleic acids are designed to be complementary to a region of mRNA expressed by a gene, so that the antisense molecule hybridizes to the mRNA, thus blocking translation of the mRNA into a polypeptide. Several classes of antisense oligonucleotide are known to those skilled in the art, including cleavers and blockers. Cleavers bind to target RNA sites and activate intracellular nucleases (e.g., Rnase H or Rnase L) that cleave the target RNA. Blockers bind to target RNA and inhibit polypeptide translation by steric hindrance of the ribosomes. Examples of blockers include nucleic acids, morpholino compounds, locked nucleic acids, and methylphosphonates (Thompson, Drug Discovery Today, 7:912-917 (2002)). Antisense oligonucleotides are useful directly as therapeutic agents, and are also useful for determining and validating gene function, for example, by gene knock-out or gene knock-down experiments. Antisense technology is further described in Lavery et al., Curr. Opin. Drug Discov Devel 6 561-569 (2003), Stephens et al., Curr. Opin. Mol Ther. 5.118-122 (2003), Kurreck, Eur. J. Biochem. 270.1628-44 (2003), Dias et al, Mol Cancer Ter. 1 -347-55 (2002), Chen, Methods Mol Med. 75:621-636 (2003), Wang et al., Curr Cancer Drug Targets 1.177-96 (2001), and Bennett, Antisense Nucleic Acid Drug. Dev. 12 215-24 (2002).

The genetic variations described herein can be used for the selection and design of antisense reagents that are specific for particular variations (e.g., particular genetic variations, or polymorphic markers in MSA with particular genetic variations). Using information about the variations described herein, antisense oligonucleotides or other antisense molecules that specifically target mRNA molecules that contain one or more variations of the disclosure can be designed. In this manner, expression of mRNA molecules that contain one or more variations of the present disclosure (markers and/or haplotypes) can be inhibited or blocked. The antisense molecules can be designed to specifically bind a particular allelic form (i.e., one or several variations (alleles and/or haplotypes)) of a target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus polypeptide expression, the molecules can be used to treat a disease or disorder, such as a neurological disorder. The methodology can involve cleavage by means of ribozymes containing nucleotide sequences complementary to one or more regions in the mRNA that attenuate the ability of the mRNA to be translated. Such mRNA regions include, for example, polypeptide-coding regions, in particular polypeptide-coding regions corresponding to catalytic activity, substrate and/or ligand binding sites, or other functional domains of a polypeptide.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev, Genet. 8: 173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a polypeptide-RNA complex to specific sites on a target mRNA, leading to cleavage of the mRNA (Thompson, Drug Discovery Today, 7:912-917 (2002)). The siRNA molecules are typically about 10-15, 20, 21, 22, or 23-25 nucleotides in length. Thus, one aspect of the disclosure relates to isolated nucleic acid sequences, and the use of those molecules for RNA interference, for example, as small interfering RNA molecules (siRNA). In some embodiments, the isolated nucleic acid sequences can be 2-30 nucleotides in length, 18-26 nucleotides in length, 19-25 nucleotides in length, 20-24 nucleotides in length, or 21, 22, or 23 nucleotides in length.

Double stranded RNA induced gene silencing can occur on at least three different levels: (i) transcription inactivation, which refers to RNA guided DNA or histone methylation; (ii) siRNA induced mRNA degradation; and (iii) mRNA induced transcriptional attenuation. It is generally considered that the major mechanism of RNA induced silencing (RNA interference, or RNAi) in mammalian cells can be mRNA degradation. RNA interference (RNAi) is a mechanism that inhibits gene expression at the stage of translation or by hindering the transcription of specific genes. Specific RNAi pathway polypeptides can be guided by the dsRNA to the targeted messenger RNA (mRNA), where they “cleave” the target, breaking it down into smaller portions that can no longer be translated into a polypeptide.

Double stranded oligonucleotides can be formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

Another pathway for RNAi-mediated gene silencing originates in endogenously encoded primary microRNA (pn-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8: 173-204 (2007)). microRNAs (miRNA) are single-stranded RNA molecules of about 21-23 nucleotides in length, which regulate gene expression. Mature miRNA molecules are partially complementary to one or more messenger RNA (mRNA) molecules, and their main function is to downregulate gene expression.

Clinical applications of RNAi include the incorporation of synthetic siRNA duplexes, which can be approximately 20-23 nucleotides in size, and can have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules typically about 20-40 nucleotides in length, in some embodiments, 27, 28, 29, 30, or 40 nucleotides in length, as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs can be substrates for in vivo processing, and in some cases provide more potent gene-silencing than shorter designs (Kim et al., Nature Biotechnol. 23:222-226 (2005); Siola et al., Nature Biotechnol. 23:227-231 (2005)). In general, siRNAs can provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23.559-565 (2006), Brummelkamp et al., Science 296. 550-553 (2002)).

Since RNAi molecules, including siRNA, miRNA, and shRNA, act in a sequence-dependent manner, variants described herein can be used to design RNAi reagents that recognize specific nucleic acids comprising specific genetic variations, alleles, and/or haplotypes, while not recognizing nucleic acid sequences not comprising the genetic variation, or comprising other alleles or haplotypes. These RNAi reagents can thus recognize and destroy the target nucleic acid sequences. As with antisense reagents, RNAi reagents can be useful as therapeutic agents (i.e., for turning off disease-associated genes or disease-associated gene variants), but can also be useful for characterizing and validating gene function (e.g., by gene knock-out or gene knock-down experiments).

Delivery of RNAi can be performed by a range of methodologies known to those skilled in the art. Methods utilizing non-viral delivery can include cholesterol, stable nucleic acid-lipid particle (SNALP), heavy-chain antibody fragment (Fab), aptamers, and nanoparticles. Viral delivery methods can include use of lentivirus, adenovirus and adeno-associated virus. The siRNA molecules can in some embodiments be chemically modified to increase their stability. This can include modifications at the 2′ position of the ribose, including 2′-O-methylpunnes and 2′-fluoropyrimidines, which provide resistance to RNase activity. Other chemical modifications are possible and known to those skilled in the art.

### Antibody-Based Therapeutics

The present disclosure embodies agents that modulate a peptide sequence or RNA expressed from a gene associated with a neurological disorder. The term “biomarker”, as used herein, can comprise a genetic variation of the present disclosure or a gene product, for example, RNA and polypeptides, of any one of the genes, for example LRRK2. A genetic variation can be one or more genetic variation listed in Table 2. Such modulating agents include, but are not limited to, polypeptides, peptidomimetics, peptoids, or any other forms of a molecule, which bind to, and alter the signaling or function associated with the a neurological disorder associated biomarker, have an inhibitory or stimulatory effect on the neurological disorder associated biomarkers, or have a stimulatory or inhibitory effect on the expression or activity of the neurological disorder associated biomarkers' ligands, for example, polyclonal antibodies and/or monoclonal antibodies that specifically bind one form of the gene product but not to the other form of the gene product are also provided, or which bind a portion of either the variant or the reference gene product that contains the polymorphic site or sites.

The present disclosure provides antibody-based agents targeting a neurological disorder associated biomarkers. The antibody-based agents in any suitable form of an antibody e.g., monoclonal, polyclonal, or synthetic, can be utilized in the therapeutic methods disclosed herein. The antibody-based agents include any target-binding fragment of an antibody and also peptibodies, which are engineered therapeutic molecules that can bind to human drug targets and contain peptides linked to the constant domains of antibodies. In some embodiments, the antibodies used for targeting a neurological disorder associated biomarkers are humanized antibodies. Methods for humanizing antibodies are well known in the art. In some embodiments, the therapeutic antibodies can comprise an antibody generated against a neurological disorder associated biomarkers described in the present disclosure, wherein the antibodies are conjugated to another agent or agents, for example, a cytotoxic agent or agents.

The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the disclosure is a molecule that binds to that polypeptide or a fragment thereof, but does not substantially bind other molecules in a nucleic acid sample, which naturally contains the polypeptide. The disclosure provides polyclonal and monoclonal antibodies that bind to a polypeptide or nucleic acid of the disclosure.

In general, antibodies of the disclosure (e.g., a monoclonal antibody) can be used to isolate a polypeptide of the disclosure by standard techniques, such as affinity chromatography or immunoprecipitation. An antibody specific for a polypeptide of the disclosure can be used to detect the polypeptide (e.g., in a cellular lysate, cell supernatant, or tissue sample) in order to evaluate the abundance and pattern of expression of the polypeptide. Antibodies can be used diagnostically, prognostically, or theranostically to monitor polypeptide levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotnazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S, or 3H. Antibodies can also be useful in pharmacogenomic analysis. In such embodiments, antibodies against variant polypeptides encoded by nucleic acids according to the disclosure, such as variant polypeptides that are encoded by nucleic acids that contain at least one genetic variation of the disclosure, can be used to identify individuals that can benefit from modified treatment modalities.

Antibodies can furthermore be useful for assessing expression of variant polypeptides in disease states, such as in active stages of a disease, or in an individual with a predisposition to a disease related to the function of the polypeptide, in particular a neurological disorder. Antibodies specific for a variant polypeptide of the present disclosure that is encoded by a nucleic acid that comprises at least one polymorphic marker or haplotype as described herein can be used to screen for the presence of the variant polypeptide, for example, to screen for a predisposition to a neurological disorder as indicated by the presence of the variant polypeptide.

Antibodies can be used in other methods. Thus, antibodies are useful as screening tools for evaluating polypeptides, such as variant polypeptides of the disclosure, in conjunction with analysis by electrophoretic mobility, isoelectric point, tryptic or other protease digest, or for use in other physical assays known to those skilled in the art. Antibodies can also be used in tissue typing. In one such embodiment, a specific variant polypeptide can be correlated with expression in a specific tissue type, and antibodies specific for the variant polypeptide can then be used to identify the specific tissue type.

An anti-LRRK2 antibody can be directed to any epitope of the LRRK2 protein. An anti-LRRK2 antibody can be directed to any LRRK2 protein sequence for example between residues 1-159, 2500-2527, 2507-2527, or 2069-2087. An anti-LRRK2 antibody can be commercially available, for example NB-300-267 or an anti-phospho-S1292-LRRK2 antibody.

### Gene Therapy

Gene therapy can be used as a therapeutic to modulate a peptide sequence or RNA expressed from a gene associated with a developmental disorder. Gene therapy involves the use of DNA as a pharmaceutical agent to treat disease. DNA can be used to supplement or alter genes within an individual's cells as a therapy to treat disease. Gene therapy can be used to alter the signaling or function associated with the a developmental disorder associated biomarker, have an inhibitory or stimulatory effect on the developmental disorder associated biomarkers, or have a stimulatory or inhibitory effect on the expression or activity of the a developmental disorder associated biomarkers' ligands. In one embodiment, gene therapy involves using DNA that encodes a functional, therapeutic gene in order to replace a mutated gene. Other forms involve directly correcting a mutation, or using DNA that encodes a therapeutic polypeptide drug (rather than a natural human gene) to provide treatment. DNA that encodes a therapeutic polypeptide can be packaged within a vector, which can used to introduce the DNA inside cells within the body. Once inside, the DNA becomes expressed by the cell machinery, resulting in the production of the therapeutic, which in turn can treat the subject's disease.

Gene therapy agents and other agents for testing therapeutics can include plasmids, viral vectors, artificial chromosomes and the like containing therapeutic genes or polynucleotides encoding therapeutic products, including coding sequences for small interfering RNA (siRNA), ribozymes, and antisense RNA, which in certain further embodiments can comprise an operably linked promoter such as a constitutive promoter or a regulatable promoter, such as an inducible promoter (e.g., IPTG inducible), a tightly regulated promoter (e.g., a promoter that permits little or no detectable transcription in the absence of its cognate inducer or derepressor), or a tissue-specific promoter. Methodologies for preparing, testing and using these and related agents are known in the art. See, e.g., Ausubel (Ed.), Current Protocols in Molecular Biology (2007 John Wiley & Sons, NY); Rosenzweig and Nabel (Eds), Current Protocols in Human Genetics (esp. Ch. 13 therein, “Delivery Systems for Gene Therapy”, 2008 John Wiley & Sons, NY); Abell, Advances in Amino Acid Mimetics and Peptidomimetics, 1997 Elsevier, N.Y. In another embodiment, gene therapy agents may encompass zinc finger nuclease (ZFN) or transcription activator-like effector nuclease (TALEN) strategies, see for example: Urnov et al. (2010), Nature Reviews Genetics 11(9):636-46; Yusa et al. (2011), Nature 478(7369):391-4; Bedell et al. (2012), Nature ePub Sep 23, PubMed ID 23000899.

As a non-limiting example, one such embodiment contemplates introduction of a gene therapy agent for treating MSA (e.g., an engineered therapeutic virus, a therapeutic agent-carrying nanoparticle, etc.) to one or more injection sites in a subject, without the need for imaging, surgery, or histology on biopsy specimens. Of course, periodic monitoring of the circulation for leaked therapeutic agent and/or subsequent analysis of a biopsy specimen, e.g., to assess the effects of the agent on the target tissue, can also be considered. A gene therapy can include a therapeutic polynucleotide administered before, after, or at the same time as any other therapy described herein. In some embodiments, therapeutic genes may include an antisense version of a biomarker disclosed herein, a sequence of a biomarker described herein, or an inhibitor of a biomarker disclosed herein.

### Methods of Treatment

Some embodiments of the present disclosure relate to methods of using pharmaceutical compositions and kits comprising agents that can inhibit one or more neurological disorder associated biomarker to inhibit or decrease neurological disorder progression. Another embodiment of the present disclosure provides methods, pharmaceutical compositions, and kits for the treatment of subjects. The term “subject” as used herein includes humans as well as other mammals. The term “treating” as used herein includes achieving a therapeutic benefit and/or a prophylactic benefit. By therapeutic benefit is meant eradication or amelioration of a condition. Also, a therapeutic benefit is achieved with the eradication or amelioration of one or more of the physiological symptoms associated a neurological disorder such that an improvement is observed in the subject, notwithstanding the fact that the subject can still be afflicted with a neurological disorder.

For embodiments where a prophylactic benefit is desired, a pharmaceutical composition of the disclosure can be administered to a subject at risk of developing a neurological disorder, or to a subject reporting one or more of the physiological symptoms of a neurological disorder, even though a screening of the condition cannot have been made. Administration can prevent a neurological disorder from developing, or it can reduce, lessen, shorten, and/or otherwise ameliorate the progression of a neurological disorder, or symptoms that develop. The pharmaceutical composition can modulate or target a neurological disorder associated biomarker. Wherein, the term modulate includes inhibition of a neurological disorder associated biomarkers or alternatively activation of a neurological disorder associated biomarkers.

Reducing the activity of one or more neurological disorder's associated biomarkers is also referred to as “inhibiting” the neurological disorder's associated biomarkers. The term “inhibits” and its grammatical conjugations, such as “inhibitory,” do not require complete inhibition, but refer to a reduction in a neurological disorder's associated biomarkers' activities. In some cases such reduction is by at least 5%, at least 10%, at least 20%, at least 30%, at least 40%, at least 50%, at least 75%, at least 90%, and can be by at least 95% of the activity of the enzyme or other biologically important molecular process in the absence of the inhibitory effect, e.g., in the absence of an inhibitor. Conversely, the phrase “does not inhibit” and its grammatical conjugations refer to situations where there is less than 20%, less than 10%, and can be less than 5%, of reduction in enzyme or other biologically important molecular activity in the presence of the agent. Further the phrase “does not substantially inhibit” and its grammatical conjugations refer to situations where there is less than 30%, less than 20%, and in some cases less than 10% of reduction in enzyme or other biologically important molecular activity in the presence of the agent.

Increasing the activity and/or function of polypeptides and/or nucleic acids found to be associated with one or more neurological disorders is also referred to as “activating” the polypeptides and/or nucleic acids. The term “activated” and its grammatical conjugations, such as “activating,” do not require complete activation, but refer to an increase in a neurological disorder associated biomarkers' activities. In some cases, such increase is by at least 5%, at least 10%, at least 20%, at least 30%, at least 40%, at least 50%, at least 60%, at least 70%, at least 75%, at least 80%, at least 85%, at least 90%, and can be by at least 95% of the activity of the enzyme or other biologically important molecular process in the absence of the activation effect, e.g., in the absence of an activator. Conversely, the phrase “does not activate” and its grammatical conjugations refer to situations where there can be less than 20%, less than 10%, and less than 5%, of an increase in enzyme or other biologically important molecular activity in the presence of the agent. Further the phrase “does not substantially activate” and its grammatical conjugations refer to situations where there is less than 30%, less than 20%, and in some cases less than 10% of an increase in enzyme or other biologically important molecular activity in the presence of the agent.

The ability to reduce enzyme activity is a measure of the potency or the activity of an agent, or combination of agents, towards or against the enzyme or other biologically important molecular process. Potency can be measured by cell free, whole cell, and/or in vivo assays in terms of IC50, Ki, and/or ED50 values. An IC50 value represents the concentration of an agent required to inhibit enzyme activity by half (50%) under a given set of conditions. A Ki value represents the equilibrium affinity constant for the binding of an inhibiting agent to the enzyme or other relevant biomolecule. An ED50 value represents the dose of an agent required to affect a half-maximal response in a biological assay. Further details of these measures will be appreciated by those of ordinary skill in the art, and can be found in standard texts on biochemistry, enzymology, and the like.

The present disclosure also includes kits that can be used to treat neurological disorders. These kits comprise an agent or combination of agents that inhibits a neurological disorder associated biomarker or a neurological disease associated biomarkers and in some cases instructions teaching the use of the kit according to the various methods and approaches described herein. Such kits can also include information, such as scientific literature references, package insert materials, clinical trial results, and/or summaries of these and the like, which indicate or establish the activities and/or advantages of the agent. Such information can be based on the results of various studies, for example, studies using experimental animals involving in vivo models and studies based on human clinical trials. Kits described herein can be provided, marketed, and/or promoted to health providers, including physicians, nurses, pharmacists, formulary officials, and the like.

### Formulations, Routes of Administration, and Effective Doses

Yet another aspect of the present disclosure relates to formulations, routes of administration and effective doses for pharmaceutical compositions comprising an agent or combination of agents of the instant disclosure. Such pharmaceutical compositions can be used to treat a neurological disorder progression and neurological disorder-associated symptoms as described above.

Compounds of the disclosure can be administered as pharmaceutical formulations including those suitable for oral (including buccal and sub-lingual), rectal, nasal, topical, transdermal patch, pulmonary, vaginal, suppository, or parenteral (including intramuscular, intra-arterial, intrathecal, intradermal, intraperitoneal, subcutaneous, and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999).

In various embodiments, the pharmaceutical composition can include carriers and excipients (including, but not limited to, buffers, carbohydrates, mannitol, polypeptides, amino acids, antioxidants, bacteriostats, chelating agents, suspending agents, thickening agents, and/or preservatives), water; oils including those of petroleum, animal, vegetable or synthetic origin, such as peanut oil, soybean oil, mineral oil, sesame oil and the like; saline solutions, aqueous dextrose and glycerol solutions, flavoring agents, coloring agents, detackifiers and other acceptable additives, adjuvants, or binders, other pharmaceutically acceptable auxiliary substances to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some cases, the pharmaceutical preparation is substantially free of preservatives. In other embodiments, the pharmaceutical preparation can contain at least one preservative. General methodology on pharmaceutical dosage forms is found in Ansel et al., Pharmaceutical Dosage Forms and Drug Delivery Systems (Lippencott, Williams, & Wilkins, Baltimore Md. (1999)). It can be recognized that, while any suitable carrier known to those of ordinary skill in the art can be employed to administer the compositions of this disclosure, the type of carrier can vary depending on the mode of administration.

A treatment agent can also be encapsulated within liposomes using well-known technology. Biodegradable microspheres can also be employed as carriers for the pharmaceutical compositions of this disclosure. Suitable biodegradable microspheres are disclosed, for example, in U.S. Pat. Nos. 4,897,268; 5,075,109; 5,928,647; 5,811,128; 5,820,883; 5,853,763; 5,814,344; and 5,942,252. In some cases, a treatment agent can be a compound.

A treatment can be administered in liposomes or microspheres (or microparticles). The treatments or their pharmaceutically acceptable salts can be provided alone or in combination with one or more other agents or with one or more other forms. For example, a formulation can comprise one or more agents in particular proportions, depending on the relative potencies of each agent and the intended indication. For example, in compositions for targeting two different targets, and where potencies are similar, about a 1:1 ratio of agents can be used. The two forms can be formulated together, in the same dosage unit e.g., in one cream, suppository, tablet, capsule, aerosol spray, or packet of powder to be dissolved in a beverage; or each form can be formulated in a separate unit, e.g., two creams, two suppositories, two tablets, two capsules, a tablet and a liquid for dissolving the tablet, two aerosol sprays, or a packet of powder and a liquid for dissolving the powder, etc.

The term “pharmaceutically acceptable salt” means those salts which retain the biological effectiveness and properties of the agents used in the present disclosure, and which are not biologically or otherwise undesirable. For example, a pharmaceutically acceptable salt does not interfere with the beneficial effect of a treatment of the disclosure in inhibiting a neurological disorder, neurological disorder associated biomarker or neurological disorder biomarker's components.

A treatment can be administered in combination with one or more other treatment, forms, and/or treatments, e.g., as described above. Pharmaceutical compositions comprising combinations of a neurological disorder associated biomarker inhibitors with one or more other active agents can be formulated to comprise certain molar ratios. For example, molar ratios of about 99:1 to about 1:99 of a neurological disorder associated biomarkers' inhibitors to the other active agent can be used. A LRRK2 inhibitor can be a neurological disorder associated biomarker inhibitor. In some subset of the embodiments, the range of molar ratios of neurological disorder's associated biomarkers' inhibitors: other active agents are selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of neurological disorder's associated biomarkers' inhibitors: other active agents can be about 1:9, and in some cases can be about 1:1. The treatments can be formulated together, in the same dosage unit e.g., in one cream, suppository, tablet, capsule, or packet of powder to be dissolved in a beverage; or each treatment can be formulated in separate units, e.g., two creams, suppositories, tablets, two capsules, a tablet and a liquid for dissolving the tablet, an aerosol spray a packet of powder and a liquid for dissolving the powder, etc.

If necessary or desirable, the treatments or combinations of treatments can be administered with still other treatments. The choice of treatments that can be co-administered with the treatment and/or combinations of treatments of the instant disclosure can depend, at least in part, on the condition being treated. For example, the treatments disclosed herein can additionally contain one or more conventional anti-inflammatory drugs, such as an NSAID, e.g., ibuprofen, naproxen, acetaminophen, ketoprofen, or aspirin.

The treatment(s) (or pharmaceutically acceptable salts, esters or amides thereof) can be administered per se or in the form of a pharmaceutical composition wherein the active agent(s) is in an admixture or mixture with one or more pharmaceutically acceptable carriers. A pharmaceutical composition, as used herein, can be any composition prepared for administration to a subject. Pharmaceutical compositions for use in accordance with the present disclosure can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The treatment(s) useful in the present disclosure, or pharmaceutically acceptable salts, esters, or amides thereof, can be delivered to a subject using a number of routes or modes of administration, including oral, buccal, topical, rectal, transdermal, transmucosal, subcutaneous, intravenous, and intramuscular applications, as well as by inhalation.

The compounds of the disclosure can be formulated for parenteral administration (e.g., by injection, for example, bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example, solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible and biodegradable, such as poly(lactic-co-glycolic)acid. These materials can be made into micro or nanospheres, loaded with drug, and further coated or derivatized to provide superior sustained release performance. Vehicles suitable for periocular or intraocular injection include, for example, suspensions of therapeutic agent in injection grade water, liposomes, and vehicles suitable for lipophilic substances. Other vehicles for periocular or intraocular injection are well known in the art.

The composition can be formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

When administration is by injection, the active compound can be formulated in aqueous solutions, specifically in physiologically compatible buffers such as Hanks' solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In some cases, the pharmaceutical composition does not comprise an adjuvant or any other substance added to enhance the immune response stimulated by the peptide. The pharmaceutical composition can comprise a substance that inhibits an immune response to the peptide. Methods of formulation are known in the art, for example, as disclosed in Remington's Pharmaceutical Sciences, latest edition, Mack Publishing Co., Easton P.

In addition to the formulations described previously, the agents can also be formulated as a depot preparation. Such long acting formulations can be administered by implantation or transcutaneous delivery (for example, subcutaneously or intramuscularly), intramuscular injection, or use of a transdermal patch. Thus, for example, the agents can be formulated with suitable polymeric or hydrophobic materials (for example, as an emulsion in an acceptable oil) or ion exchange resins, or as sparingly soluble derivatives, for example, as a sparingly soluble salt.

### Kits

Kits useful in the methods of the disclosure comprise components useful in any of the methods described herein, including for example, primers for nucleic acid amplification, hybridization probes for detecting genetic variation, or other marker detection, restriction enzymes, nucleic acid probes, optionally labeled with suitable labels, allele-specific oligonucleotides, antibodies that bind to an altered polypeptide encoded by a nucleic acid of the disclosure as described herein or to a wild type polypeptide encoded by a nucleic acid of the disclosure as described herein, means for amplification of genetic variations or fragments thereof, means for analyzing the nucleic acid sequence of nucleic acids comprising genetic variations as described herein, means for analyzing the amino acid sequence of a polypeptide encoded by a genetic variation, or a nucleic acid associated with a genetic variation, etc. The kits can, for example, include necessary buffers, nucleic acid primers for amplifying nucleic acids, and reagents for allele-specific detection of the fragments amplified using such primers and necessary enzymes (e.g., DNA polymerase). Additionally, kits can provide reagents for assays to be used in combination with the methods of the present disclosure, for example, reagents for use with other screening assays for a neurological disorder.

The disclosure pertains to a kit for assaying a sample from a subject to detect the presence of a genetic variation, wherein the kit comprises reagents necessary for selectively detecting at least one particular genetic variation in the genome of the individual. In some aspects, the disclosure pertains to a kit for assaying a sample from a subject to detect the presence of at least particular allele of at least one polymorphism associated with a genetic variation in the genome of the subject. In some aspects, the reagents can comprise at least one contiguous oligonucleotide that hybridizes to a fragment of the genome of the individual comprising at least genetic variation. In some aspects, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one genetic variation, or a fragment of a genetic variation. Such oligonucleotides or nucleic acids can be designed using the methods described herein. In some aspects, the kit comprises one or more labeled nucleic acids capable of allele-specific detection of one or more specific polymorphic markers or haplotypes with a genetic variation, and reagents for detection of the label. In some aspects, a kit for detecting SNP markers can comprise a detection oligonucleotide probe, that hybridizes to a segment of template DNA containing an SNP polymorphisms to be detected, an enhancer oligonucleotide probe, detection probe, primer and/or an endonuclease, for example, as described by Kutyavin et al. (Nucleic Acid Res. 34:e128 (2006)).

The DNA template is amplified by any means of the present disclosure, prior to assessment for the presence of specific genetic variations as described herein. Standard methods well known to the skilled person for performing these methods can be utilized, and are within scope of the disclosure. In one such embodiment, reagents for performing these methods can be included in the reagent kit.

In a further aspect of the present disclosure, a pharmaceutical pack (kit) is provided, the pack can comprise a therapeutic agent and a set of instructions for administration of the therapeutic agent to humans screened for one or more variants of the present disclosure, as disclosed herein. The therapeutic agent can be a small molecule drug, an antibody, a peptide, an antisense or RNAi molecule, or other therapeutic molecules as described herein. In some aspects, an individual identified as a carrier of at least one variant of the present disclosure is instructed to take a prescribed dose of the therapeutic agent. In one such embodiment, an individual identified as a carrier of at least one variant of the present disclosure is instructed to take a prescribed dose of the therapeutic agent. In some aspects, an individual identified as a non-carrier of at least one variant of the present disclosure is instructed to take a prescribed dose of the therapeutic agent.

Also provided herein are articles of manufacture, comprising a probe that hybridizes with a region of human chromosome as described herein and can be used to detect a polymorphism described herein. For example, any of the probes for detecting polymorphisms described herein can be combined with packaging material to generate articles of manufacture or kits. The kit can include one or more other elements including: instructions for use and other reagents such as a label or an agent useful for attaching a label to the probe. Instructions for use can include instructions for screening applications of the probe for making a diagnosis, prognosis, or theranosis to a neurological disorder in a method described herein. Other instructions can include instructions for attaching a label to the probe, instructions for performing in situ analysis with the probe, and/or instructions for obtaining a nucleic acid sample to be analyzed from a subject. The kit can include a labeled probe that hybridizes to a region of human chromosome as described herein.

The kit can also include one or more additional reference or control probes that hybridize to the same chromosome or another chromosome or portion thereof that can have an abnormality associated with a particular endophenotype. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes. Kits for use in self-testing can also be provided. Such test kits can include devices and instructions that a subject can use to obtain a nucleic acid sample (e.g., buccal cells, blood) without the aid of a health care provider. For example, buccal cells can be obtained using a buccal swab or brush, or using mouthwash.

Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the nucleic acid sample can be coded, for example, with a bar code for identifying the subject who provided the sample.

An in vitro screening test can comprise one or more devices, tools, and equipment configured to collect a sample from an individual. In some aspects of an in vitro screening test, tools to collect a sample can include one or more of a swab, a scalpel, a syringe, a scraper, a container, and other devices and reagents designed to facilitate the collection, storage, and transport of a sample. In some aspects, an in vitro screening test can include reagents or solutions for collecting, stabilizing, storing, and processing a nucleic acid sample.

Such reagents and solutions for nucleotide collecting, stabilizing, storing, and processing are well known by those of skill in the art and can be indicated by specific methods used by an in vitro screening test as described herein. In some aspects, an in vitro screening test, as disclosed herein, can comprise a microarray apparatus and reagents, a flow cell apparatus and reagents, a multiplex nucleotide sequencer and reagents, and additional hardware and software necessary to assay a nucleic acid sample for certain genetic markers and to detect and visualize certain genetic markers.

The present disclosure further relates to kits for using antibodies in the methods described herein. This includes, but is not limited to, kits for detecting the presence of a variant polypeptide in a test sample. One embodiment comprises antibodies such as a labeled or labelable antibody and a compound or agent for detecting variant polypeptides in a sample, means for determining the amount or the presence and/or absence of variant polypeptide in the sample, and means for comparing the amount of variant polypeptide in the nucleic acid sample with a standard, as well as instructions for use of the kit. In certain embodiments, the kit can further comprise a set of instructions for using the reagents comprising the kit.

It should be understood that the following examples should not be construed as being limiting to the particular methodology, protocols, and compositions, etc., described herein and, as such, can vary. The following terms used herein is for the purpose of describing particular embodiments only, and is not intended to limit the scope of the embodiments disclosed herein.

### Methods of Screening for Treatment Compounds

The present invention further provides methods of screening compounds for the treatment of neurodegenerative diseases, for example compounds that modulate LRRK2, PARK2, PARK7, PINK1, GBA, RAB7L1, EIF4G1, ATP13A2, SNCA, PARKIN, DJ1, PLA2G6, FBX07, GIGYF2, HTRA2, MAPT, BST1, GAK, APP, PS1, PS2, SOD1, P102L, E200K, PLA2G6, PANK2, FTL OR, UCHL1 gene or LRRK2, PARK2, PARK7, PINK1, GBA, RAB7L1, EIF4G1, ATP13A2, SNCA, Parkin, DJ1, PLA2G6, FBX07, GIGYF2, HTRA2, or, MAPT, BST1, GAK, APP, PS1, PS2, SOD1, P102L, 6-OPRI, E200K, PLA2G6, PANK2, FTL, or UCHL1 protein. In some cases, a protein or gene mutation can be a prion protein or gene mutation. It is understood by one of skill in the art that the examples provided herein can be with reference to the genes or proteins disclosed herein.

The present invention provides methods of screening compounds for the treatment of neurodegenerative diseases, for example LRRK2 inhibitors. The screening method can comprise monitoring LRRK2 autophosphorylation in the presence and absence of a compound. A compound that reduces, prevents, or otherwise inhibits LRRK2 autophosphorylation in comparison to autophosphorylation in the absence of such compound (and optionally in comparison to positive and other negative controls) is indicative that the compound is LRRK2 inhibitor or a potential treatment for a neurodegenerative disease disclosed herein. The screening method can comprise monitoring LRRK2 phosphorylation of another protein (e.g., myelin basic protein (“MBP”)) in the presence and absence of a compound. A compound that reduces, prevents or otherwise inhibits LRRK2 phosphorylation of MBP in comparison to phosphorylation of MBP in the absence of such compound (and optionally in comparison to positive and other negative controls) is indicative that the compound is an LRRK2 inhibitor. The methods may further comprise monitoring LRRK2 autophosphorylation and phosphorylation of MBP in the presence and absence of a compound to determine specificity of such inhibitors.

The disclosed methods further contemplate in vitro methods of screening compounds for the treatment of neurodegenerative diseases, for example MSA. More specifically, the present invention provides methods for determining whether a compound can attenuate toxicity induced by LRRK2 overexpression and/or LRRK2 mutant expression. In particular embodiments, cultures, for example primary cultures (cortical neurons or glia cells), can be transiently transfected with wild-type or LRRK2 mutants (e.g., G2019S, I1371V) and neuronal/glia toxicity is monitored in the presence and absence of a compound. Compounds that protect against wild-type and/or LRRK2 mutant (e.g., I1371V) toxicity can be identified as putative treatment for neurodegenerative diseases, for example MSA. The invention further contemplates screening cells, for example primary cells reprogrammed into induced pluripotent stem cells and further differentiated into various brain cells, e.g. neurons, astrocytes, oligodendrocytes, glia, and primary cells that has transdifferentiated into various brain cells, e.g. neurons, astrocytes, oligodendrocytes, glia.

The present invention further relates to transgenic models. More specifically, the present invention relates to transgenic models of human LRRK2 expression. In particular embodiments, the transgenic models of the present invention express a wild-type human LRRK2 protein. Amino acid sequences of wild type human LRRK2 proteins are known in the art. For example GenBank Accession NO. NP-940980. In certain aspects, the transgenic models of the present invention express a mutant human LRRK2 protein, for example the LRRK2 protein mutants disclosed herein. The mutation can be a substitution including, but not limited, one or more substitutions at the following positions: I1371, N551, 1723, R1398, R1441, R1514, P1542, R1628, M1646, S1647, M1869, G2019, G2385, or T2397.The substitution mutation can include, but is not limited, to the following: I1371V, N551K, I723V, R1398H, R1441C, R1441G, R1441H, R1514Q, P1542S, R1628P, M1646T, S1647T, M1869T, G2019S, G2385R, or T2397M. The mutation can be at the I1371 position and can be an I1371V mutation. The mutation can be a mutation as listed in Table 1 or Table 2. The transgenic animals of the present invention, which express a mutant human LRRK2 protein, can exhibit one or more cardinal phenotypes of a neurodegenerative disease disclosed herein. The term “animal” can refer to any animal (e.g., a mammal) including, but not limited to, humans, non-human primates, rodents (e.g., mice, rats, etc.), and the like. In particular embodiments, the present invention comprises a transgenic mouse. The term “transgenic” is used in its ordinary sense, includes germline and non-germline expression of transgenes in animals, and further includes the expression of a gene in one or more cells of an animal.

In some instances, a transgenic non-human mammal genome can comprise a human wild-type LRRK2 gene. The present invention further provides a transgenic non-human mammal whose genome comprises a human LRRK2 genetic variation, wherein expression of the gene creates a neurodegenerative disease like phenotype, for example a MSA-like phenotype. The LRRK2 genetic variation can be an LRRK2 mutation at position I1371.

In some cases, an expression of an LRRK2 genetic variation can be via the Herpes Simplex Virus Amplicon expression and delivery platform. A transgenic non-human mammal of the present invention may be a Herpes Simplex Virus (“HSV”) amplicon-based model of LRRK2 that exhibits Parkinson's or MSA-like phenotypes. The transgenic non-human mammal can be an HSV amplicon-based model of LRRK2 dopaminergic neurotoxicity or glia toxicity. The transgenic mammals of the present invention can be used to screen for compounds that modulate LRRK2 activity. The transgenic mammals can be used to test whether compounds inhibits LRRK2 and rescue or protect against one or more MSA-like phenotypes. In a specific embodiment, the transgenic mammals may be used to test whether a candidate LRRK2 inhibitor is protective against loss of dopamine neurons. The present invention provides methods for screening compounds for the ability to modulate LRRK2 activity in a transgenic nonhuman mammal and reduce changes associated with a neurodegenerative disease-like phenotype induced by LRRK2 transgene expression. The method may comprise exposing the transgenic non-human mammal to an effective amount of a compound to modulate activity of the LRRK2 protein, and determining whether the compound has a significant effect on the neurodegenerative disease-like phenotype of the transgenic non-human mammal as compared to a transgenic non-human mammal expressing wild-type or mutant LRRK2 that was not exposed to the compound. A compound that has an effect on the neurodegenerative disease-like phenotype of the transgenic non-human mammal induced by activity of the expressed LRRK2 protein can be identified as a compound for modulating activity of the LRRK2 protein.

The method can comprise exposing the transgenic non-human mammal to an environmental stressor to accelerate expression of a neurodegenerative disease-like phenotype, exposing the transgenic non-human mammal to an effective amount of a compound to modulate activity of the LRRK2 protein, and determining whether the compound has a significant effect on the neurodegenerative disease-like phenotype of the transgenic non-human mammal as compared to a transgenic non-human mammal expressing wild-type or mutant LRRK2 that was not exposed to the candidate compound. The environmental stressor can be any known stressor associated with a neurodegenerative disease, and includes any stressor that accelerates a neurodegenerative disease-like phenotype resulting from LRRK2 expression. Environmental stressors may include, but are not limited to, oxidative stress, insecticides, 1-methyl-4-phenyl-1,2,3,6-tetrahydropyridine, Nitro oxide (NO) donor, proteasome inhibitors, endocrine conditions, stroke, hypertension, diabetes, smoking, head trauma, depression, infection, tumors, vitamin deficiencies, immune and metabolic conditions, and chemical exposure.

In one aspect the transgenic model can be a transgenic nematode model. The nematode can belong to the subgenus Caenorhabditis. The nematode can be Caenorhabditis elegans (“C. elegans”). The invention can provide for a transgenic nematode whose genome comprises a human wild-type LRRK2 gene. The present invention further provides a transgenic nematode whose genome comprises a human LRRK2 genetic variation, wherein expression of the gene creates a neurodegenerative disease-like phenotype. The present invention provides methods for screening compounds for the ability to modulate LRRK2 activity in a transgenic nematode and reduce changes associated with the neurodegenerative disease-like phenotype induced by LRRK2 transgene expression. The method may comprise exposing the transgenic nematode to an effective amount of a compound to modulate activity of the LRRK2 protein, and determining whether the compound has a significant effect on the neurodegenerative disease -like phenotype of the nematode as compared to a nematode expressing wild-type or mutant LRRK2 that was not exposed to the compound. A compound that has an effect on the neurodegenerative disease-like phenotype of the transgenic nematode induced by activity of the expressed LRRK2 protein can be identified as a candidate compound for modulating activity of the LRRK2 protein.

In another aspect, the methods described herein can be used to identify compound that modulate the expression levels of any of the genes that causes an LRRK2 mutation listed in Table 1 or Table 2. In some aspects, a compound can modulate the genes that cause an LRRK2 mutation listed in Table 1 or Table 2 by at least 10%, 20%, 30%, 40%, 50%, 60%, 70%, 80%, 90%, 95%, or 100% relative to an untreated control. According to one approach, compounds can be added at varying concentrations to the culture medium of cells expressing the polypeptide of Table 1 or Table 2. Gene expression of the polypeptide can then be measured, for example, by standard Northern blot analysis using any appropriate fragment prepared from the nucleic acid molecule encoding the polypeptide as a hybridization probe or by real time PCR with appropriate primers, or methods disclosed herein. The level of gene expression in the presence of the compound can be compared to the level measured in a control culture medium lacking the compound. If desired, the effect of compounds may, in the alternative, be measured at the protein level using the same general approach and standard immunological techniques, such as Western blotting or immunoprecipitation with an antibody specific to the polypeptide for example. One of skill in the art would appreciate that any method disclosed herein can be used to detect gene expression and protein expression levels. For example, immunoassays may be used to detect or monitor the level of the polypeptide from Table 1 or Table 2. Polyclonal or monoclonal antibodies which are capable of binding to such polypeptides may be used in any standard immunoassay format (e.g., ELISA or RIA assay) to measure protein levels of the polypeptide. The polypeptides can also be measured using mass spectroscopy, high performance liquid chromatography, spectrophotometric or fluorometric techniques, or combinations thereof.

In another case, expression of a reporter gene that is operably linked to the promoter of a gene that causes an LRRK2 mutation listed in Tables 1 or 2, can also be used to identify a compound for treating or preventing a neurodegenerative disease, for example MSA. Assays employing the detection of reporter gene products are sensitive and readily amenable to automation, hence making them ideal for the design of high-throughput screens. Assays for reporter genes may employ, for example, calorimetric, chemiluminescent, or fluorometric detection of reporter gene products. Many varieties of plasmid and viral vectors containing reporter gene cassettes are easily obtained. Such vectors contain cassettes encoding reporter genes such as lacZ/β-galactosidase, green fluorescent protein, and luciferase, among others. A genomic DNA fragment carrying a selected transcriptional control region (e.g., a promoter and/or enhancer) can be first cloned using standard approaches. The DNA carrying the selected transcriptional control region is then inserted, by DNA subcloning, into a reporter vector, thereby placing a vector-encoded reporter gene under the control of that transcriptional control region. The activity of the selected transcriptional control region operably linked to the reporter gene can then be directly observed and quantified as a function of reporter gene activity in a reporter gene assay. In one embodiment, for example, the transcriptional control region could be cloned upstream from a luciferase reporter gene within a reporter vector. This could be introduced into the test cells, along with an internal control reporter vector (e.g., a lacZ gene under the transcriptional regulation of the β-actin promoter). After the cells are exposed to the test compounds, reporter gene activity can be measured and the reporter gene activity is normalized to internal control reporter gene activity. By “operably linked” is meant that a nucleic acid molecule and one or more regulatory sequences (e.g., a promoter) are connected in such a way as to permit expression of the gene product (i.e., RNA) when the appropriate molecules (e.g., transcriptional activator proteins) are bound to the regulatory sequences.

In another case, a compound can be tested for its ability to modulate the biological activity of one or more LRRK2 polypeptides mutations listed in Table 1 or Table 2 in cells that naturally express such a polypeptide, after transfection with a cDNA for this polypeptide, or in cell-free solutions containing the polypeptide. Accordingly, compounds can be first contacted with a polypeptide from either table, having some level of a characteristic biological activity (including cell survival). The exact level of activity is unimportant and may be at least 10%, 20%, 30%, 40%, 50%, 60%, 70%, 80%, 90%, 100%, or more than 100% of the biological activity of the naturally-occurring, wild-type polypeptide. The effect of a compound on the activity of the polypeptide can be tested by radioactive and non-radioactive binding assays, competition assays, and receptor signaling assays.

### EXAMPLES

### Example 1

### LRRK2 I1371V Mutation

168 patient DNA and 30 brainbank DNA samples were sequenced. 188 genes were fully sequenced. One subject with MSA by clinical history and by autopsy who had a maternal aunt with Parkinson's disease was found to have a mutation in the LRRK2 gene. The LRRK2 genetic variation detected in the subject with MSA causes an LRRK2 I1371V mutation.

### Example 2

### Genotyping

A sample containing nucleic acids can be obtained from a subject. Genotyping can be performed on a Sequenom MassArray iPLEX platform. Primer sequence for use can be found in Table 2. Positive control DNA can be included for each variant; where positive genomic control DNA is unavailable, a synthetic positive control DNA sequence can be generated by a mismatch primer PCR method. Direct DNA sequencing can be employed to confirm genotyping for all variants.

### Example 3

### Case study: Identifying p.Ile 1371Val (LRRK2 I1371V) Genetic Variant in a Case with Clinically Diagnosed and Pathologically Proven MSA

Sample ascertainment and DNA isolation: From the brain donation program at the Parkinson's Institute and Clinical Center, 26 brains with family history and a with clinicopathological diagnosis of PD (n=20), MSA (n=4), or PSP (progressive supranuclear palsy) (n=2) were selected. DNA was extracted from fresh frozen brain tissue with Qiagen DNeasy Blood & Tissue Kit or from formalin-fixed paraffin embedded tissue with Gentra Puregene Tissue Kit according to manufacturer's instructions

188-gene panel sequencing: A targeted 188-gene panel including genes that have been described to harbor pathogenic mutations for or that were associated with Parkinson's disease, amyotrophic lateral sclerosis, and Alzheimer disease was used, and all exons and exon-intron boundaries (Table 4) were sequenced. Agilent SureSelect™ ® was used for target gene enrichment, and next-generation sequencing was performed on Illumina's Miseq™ ® instrumentation. The raw data were aligned using GATK workflow for pre-processing, variant discovery, and call-set refinement. After variant processing, sequence variants were annotated using Variant Effect Predictor toolkit. The LRRK2 I1371V mutation was confirmed by bi-directional Sanger sequencing of amplified PCR products from genomic DNA.

TABLE 4
188-gene panel for neurodegenerative diseases

| Gene name | Chrom. | Gene Range | Gene name | Chrom. | Gene Range |
|---|---|---|---|---|---|
| APOE | 19 | 45409039 . . . 45412650 | TMEM43 | 3 | 14124940 . . . 14143680 |
| APP | 21 | 27252861 . . . 27543446 | TTYH3 | 7 | 2631969 . . . 2664802 |
| PSEN2 | 1 | 227058273 . . . 227083804 | FAM73B | 9 | 129036620 . . . 129072082 |
| TREM2 | 6 | 41126246 . . . 41130922 | NAV2 | 11 | 19350724 . . . 20121601 |
| ANG | 14q11.2 | 21152336 . . . 21162345 | TMEM67 | 8 | 93754844 . . . 93819234 |
| C21orf2 | 21 | 44328944 . . . 44339402 | ACBD5 | 10 | 27195214 . . . 27242275 |
| CAMK2A | 5 | 150219491 . . . 150289840 | VPS26B | 11 | 134224667 . . . 134247792 |
| DAO | 12 | 108880030 . . . 108900934 | CYGB | 17 | 76527348 . . . 76537905 |
| DCTN1 | 2 | 74588281 . . . 74619214 | OSBPL1A | 18 | 24162045 . . . 24397882 |
| DDX6 | 11 | 118747763 . . . 118791263 | OSBPL6 | 2 | 178194481 . . . 178399433 |
| DNMT3A | 2 | 25228552 . . . 25342590 | OSBPL9 | 1 | 51616874 . . . 51789219 |
| FUS | 16p11.2 | 31191431 . . . 31206192 | OSBPL10 | 3 | 31660825 . . . 31981850 |
| HFE | 6 | 26087509 . . . 26095469 | OSBPL11 | 3 | 125528858 . . . 125595537 |
| hnRNPA1 | 12 | 54280696 . . . 54287087 | ZNF837 | 19 | 58367623 . . . 58381022 |
| hnRNPA2B1 | 7 | 26189927 . . . 26200793 | FRA10AC1 | 10 | 93667883 . . . 93702572 |
| LCN 2 | 9 | 128149453 . . . 128153455 | FAM82A1 | 2 | 37925319 . . . 38067142 |
| NEFH | 22 | 29876181 . . . 29887279 | MBOAT1 | 6 | 20099684 . . . 20212464 |
| NEK1 | 4 | 169393270 . . . 169612629 | VPS13B | 8 | 99011179 . . . 99878219 |
| NSF | 17 | 46590669 . . . 46757464 | TTC39B | 9 | 15170844 . . . 15307360 |
| OSBP | 11 | 59574398 . . . 59616144 | MOSPD2 | X | 14873405 . . . 14922166 |
| PRODH | 22 | 18912774 . . . 18936553 | ZNF296 | 19 | 45071500 . . . 45076430 |
| PRPH | 12 | 49688909 . . . 49692481 | C9ORF72 | 9p21.2 | 27546544 . . . 27573S42 |
| PYGM | 11 | 64746389 . . . 64760715 | GRID2IP | 7 | 6496778 . . . 6551436 |
| RAB2A | 8 | 60516910 . . . 60623644 | MAPT | 17q21.31 | 43971748 . . . 44105700 |
| ATXN1 | 6 | 16299112 . . . 16761490 | PSEN1 | 14q24.2 | 73603143 . . . 73690399 |
| ATXN2 | 12q24.12 | 111890018 . . . 112037480 | TRPM7 | 15q21.2 | 50849351 . . . 50979012 |
| SDF2 | 17 | 28648356 . . . 28662189 | AFF2 | X | 148500619 . . . 149000663 |
| SLC1A5 | 19 | 46760328 . . . 46789019 | EPHA4 | 2 | 221418027 . . . 221572290 |
| SOD1 | 21 | 33031935 . . . 33041244 | USP14 | 18 | 158483 . . . 213739 |
| SPAST | 2 | 32063592 . . . 32157637 | HDAC6 | X | 48660487 . . . 48683380 |
| TFRC | 3 | 196049284 . . . 196082161 | PFN1 | 17 | 4848945 . . . 4852381 |
| TIAL1 | 10 | 119573465 . . . 119597029 | APC | 5 | 112707505 . . . 112846239 |
| TTC1 | 5 | 160009100 . . . 160065545 | BST1 | 4 | 15702950 . . . 15739498 |
| UBE2D2 | 5 | 139561166 . . . 139628434 | CALM1 | 14 | 90396983 . . . 90408275 |
| VCP | 9 | 35056065 . . . 35072739 | CALM3 | 19 | 46601255 . . . 46610782 |
| TAF15 | 17 | 35809455 . . . 35847242 | SCARB2 | 4 | 76158737 . . . 76213899 |
| PABPC4 | 1 | 39560813 . . . 39576849 | CDK3 | 17 | 76000906 . . . 76005999 |
| VAPB | 20q13.32 | 56964175 . . . 57026157 | CHRNA3 | 15 | 78593052 . . . 78621295 |
| FADS2 | 11 | 61816203 . . . 61867354 | CSNK1E | 22 | 38290691 . . . 38318084 |
| EIF2AK3 | 2 | 88556741 . . . 88627576 | CSNK1G3 | 5 | 123512099 . . . 123617045 |
| PITPNM1 | 11 | 67491768 . . . 67505372 | ECT2 | 3 | 172750646 . . . 172829265 |
| ZNF432 | 19 | 52033424 . . . 52048820 | EIF4G1 | 3 | 184314495 . . . 184335358 |
| MATR3 | 5 | 139273752 . . . 139331677 | ACSL3 | 2 | 222861014 . . . 222943401 |
| OSBPL2 | 20 | 62238485 . . . 62296213 | ACSL4 | X | 109641335 . . . 109733392 |
| FIG4 | 6q21 | 110012424 . . . 110146634 | GAK | 4 | 849277 . . . 932390 |
| OPTN | 10p13 | 13142082 . . . 13180276 | GAPDH | 12 | 6534419 . . . 6538371 |
| UBE4B | 1 | 10032983 . . . 10181239 | GART | 21 | 33503931 . . . 33542917 |
| PAICS | 4 | 56410516 . . . 56461368 | GBA | 1 | 155204239 . . . 155214653 |
| RCAN3 | 1 | 24502351 . . . 24537020 | GCH1 | 14 | 54842005 . . . 54902885 |
| FAF1 | 1 | 50441263 . . . 50960264 | HSPA1L | 6 | 31809619 . . . 31821040 |
| PRAF2 | X | 49071156 . . . 49074045 | HSPA5 | 9 | 125234848 . . . 125241387 |
| RAB3GAP1 | 2 | 135052265 . . . 135176394 | HSPA8 | 11 | 123057492 . . . 123062335 |
| LSD1 | 1 | 23019443 . . . 23083691 | INSRR | 1 | 156840873 . . . 156858920 |
| WDTC1 | 1 | 27234516 . . . 27308633 | KCNA3 | 1 | 110653560 . . . 110675033 |
| SETX | 9 | 132261440 . . . 132356726 | RAB8A | 19 | 16111680 . . . 16133635 |
| VPS13A | 9 | 77177353 . . . 77417483 | NDUFV2 | 18 | 9102630 . . . 9134345 |
| BICD2 | 9 | 92711363 . . . 92764801 | NEDD4 | 15 | 55826917 . . . 55993746 |
| ARHGEF12 | 11 | 120336555 . . . 120489936 | PARK2 | 6 | 161768590 . . . 163148834 |
| PUM2 | 2 | 20248692 . . . 20350975 | PDE1C | 7 | 31752052 . . . 32299404 |
| TARDBP | 1p36.22 | 11072679 . . . 11085549 | PDE4B | 1 | 65792510 . . . 66374579 |
| PPP1R15A | 19 | 48872392 . . . 48876062 | PDE8A | 15 | 84980513 . . . 85139145 |
| OSBP2 | 22 | 30693782 . . . 30907824 | PPM1A | 14 | 60245752 . . . 60299087 |
| RAB3GAP2 | 1 | 220148268 . . . 220272501 | PPP1R3C | 10 | 91628440 . . . 91633101 |
| PAMR1 | 11 | 35431827 . . . 35530300 | PTPN9 | 15 | 75467121 . . . 75579291 |
| CHMP2B | 3 | 87227263 . . . 87255548 | RPS21 | 20 | 62387065 . . . 62388520 |
| OSBPL3 | 7 | 24796537 . . . 24980212 | SNCA | 4 | 90645250 . . . 90759447 |
| UBXN7 | 3 | 196353490 . . . 196432474 | TBCA | 5 | 77691170 . . . 77776361 |
| PABPC1 | 8 | 100702916 . . . 100722482 | UBE2D3 | 4 | 102794383 . . . 102868893 |
| UBQLN2 | Xp11.21 | 56590026 . . . 56593443 | UBE2E2 | 3 | 23203007 . . . 23590805 |
| SH3KBP1 | X | 19533965 . . . 19887626 | UBE2H | 7 | 129830732 . . . 129952960 |
| GPN3 | 12 | 110452486 . . . 110468829 | UCHL1 | 4 | 41256881 . . . 41268429 |
| VPS29 | 12 | 110491523 . . . 110502140 | UPP1 | 7 | 48088603 . . . 48108733 |
| TMEM106B | 7 | 12211203 . . . 12237264 | PLA2G6 | 22 | 38111495 . . . 38192051 |
| VPS13C | 15 | 61852389 . . . 62060465 | KLF11 | 2 | 10042993 . . . 10054836 |
| STX17 | 9 | 99906633 . . . 99974536 | RAB7L1 | 1 | 205737114 . . . 205744610 |
| FAM82A2 | 15 | 40735887 . . . 40755260 | PUM1 | 1 | 30931506 . . . 31065717 |
| LSG1 | 3 | 194640788 . . . 194672477 | NFAT5 | 16 | 69565966 . . . 69704666 |
| KIF16B | 20 | 16272097 . . . 16573434 | TREH | 11 | 118658232 . . . 118679672 |
| SPPL2B | 19 | 2328630 . . . 2355102 | PARK7 | 1 | 7961654 . . . 7985282 |
| MICAL3 | 22 | 17787649 . . . 18024559 | INPP5F | 10 | 119726047 . . . 119829151 |
| PITPNM2 | 12 | 122983480 . . . 123110488 | SREBF | 3 | 47413694 . . . 47476113 |
| ALS2 | 2 | 201700263 . . . 201781172 | SARM1 | 17 | 28371662 . . . 28401045 |
| HIVEP3 | 1 | 41506365 . . . 42035925 | NEDD4L | 18 | 58044355 . . . 58401540 |
| RNF123 | 3 | 49689517 . . . 49721529 | ATP13A2 | 1 | 16985958 . . . 17011972 |
| MRPL9 | 1 | 151759643 . . . 151763564 | SIRT1 | 10 | 67884669 . . . 67918390 |
| PINK1 | 1 | 20959948 . . . 20978004 | FBXO7 | 22 | 32474720 . . . 32498831 |
| IRX6 | 16 | 55324494 . . . 55330760 | RNF11 | 1 | 51236273 . . . 51273447 |
| USP38 | 4 | 143184917 . . . 143221988 | STK39 | 2 | 167954020 . . . 168247595 |
| GPNMB | 7 | 23246686 . . . 23275110 | PDE11A | 2 | 177623249 . . . 178108339 |
| OSBPL5 | 11 | 3087116 . . . 3165352 | SLC35C2 | 20 | 46347350 . . . 46364458 |
| LRRK2 | 12 | 40618813 . . . 40763087 | SIRT7 | 17 | 81911939 . . . 81918182 |
| PM20D1 | 1 | 205828022 . . . 205850148 | FBXW7 | 4 | 152321258 . . . 152535241 |
| YTHDF3 | 8 | 63168553 . . . 63212788 | VPS35 | 16 | 46659677 . . . 46689232 |
| MMP9 | 20 | 46008908 . . . 46016561 | MCCC1 | 3 | 183015218 . . . 183099585 |

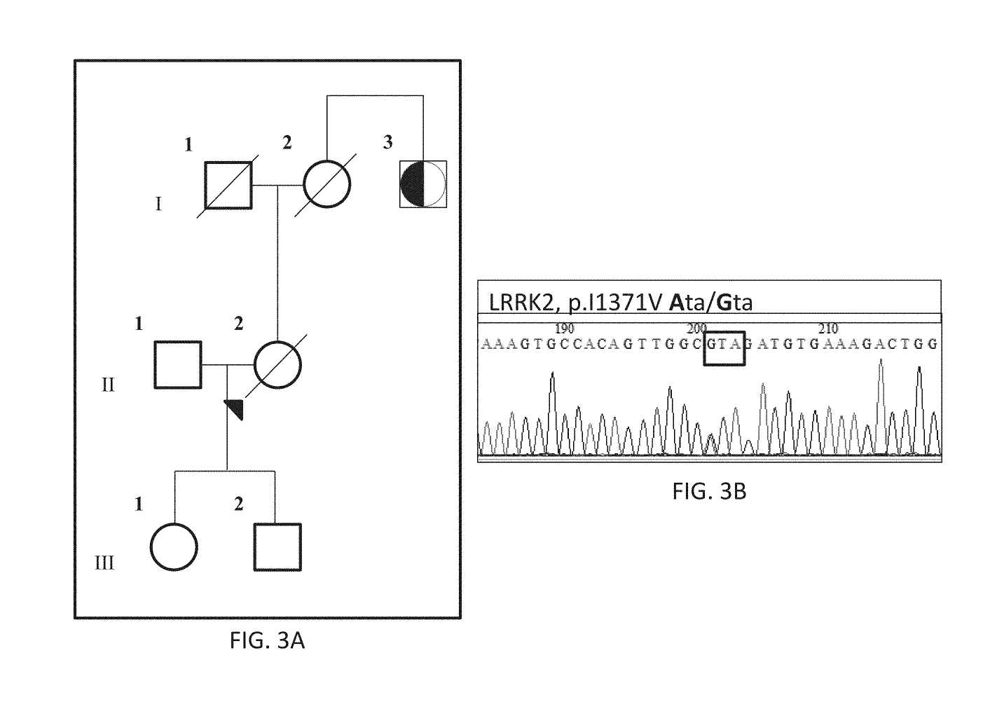

Results and case description: Next-generation sequencing of familial cases with Parkinsonism revealed an LRRK2, I1371V variant in a case with pathologically confirmed MSA showing primarily olivopontocerebellar involvement (FIG. 3B).

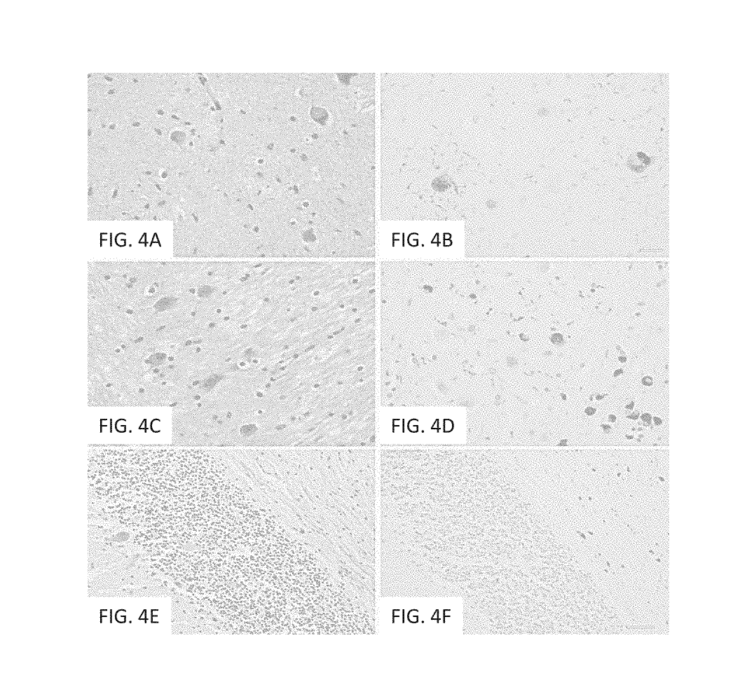

Patient history and neuropathological presentation: At the age of 50, the patient started experiencing balance problems, slurred speech, and changes in her hand writing. Her disease progressed quickly over a year and a half with severe balance problems resulting in multiple falls, bladder urgency, anxiety attacks, and depression. L-Dopa treatment did not improve her symptoms. On examination, she arose from a chair slowly, walked with ataxic broad-based gait and without assistance, but with a threat of falling. Finger-to-nose and heel to shin testing were ataxic with mild side to side dysmetria including frequent overshoot ataxia. Rapid alternating movements were slowed bilaterally (left greater than right). No rigidity and no resting, postural, or kinetic tremor were noted. Strength was normal, reflexes slightly brisk, but no pathological reflexes or dystonia noted. A MRI was reported to be normal. Symptoms deteriorated over the course of three years and she passed away at age 53. Her maternal aunt was reported to have had Parkinsonism. At autopsy, multiple system atrophy (olivopontocerebellar degeneration) was neuropathologically described. The neocortex showed no neuronal loss, vacuolar changes, or gliosis. The hippocampus, nucleus basalis of Meynert, and hypothalamus had a normal neuronal population, no neuronal cytoplasmic inclusions (NCIs), but exhibited glial cytoplasmic inclusions (GCIs). By alpha-synuclein immunohistochemistry many GCIs, few NCIs and dystrophic neurites were detected throughout the basal ganglia, especially in white matter tracts in the lateral putamen. There was neuronal loss in the substantia nigra with extraneuronal neuromelanin, gliosis, numerous GCIs, and few NCIs. Furthermore, GCIs were frequent in the midbrain gray matter and cerebral peduncle. The inferior olivary nucleus had neuronal loss and gliosis with many NCIs and dystrophic neurites (FIG. 4A, 4B). No Lewy bodies were detected, but neuronal loss and extraneuronal neuromelanin were observed. The pontine nuclei had numerous NCIs and neuronal intranuclear inclusions (NIIs) and alpha-synuclein positive dystrophic neurites (FIG. 4C, 4D). There was cerebellar Purkinje cell loss with Bergmann gliosis and Purkinje cell axonal torpedoes, with extensive demyelination in the cerebellar white matter with many GCIs (FIG. 4E, 4F). No senile plaques or neurofibrillary tangles were detected in the neocortex, hippocampus, amygdala, basal nucleus of Meynert, basal ganglia, or hypothalamus.

Discussion: Reported is a rare LRRK2 variant (LRRK2, I1371V) with a clinical and neuropathological presentation of MSA with primarily olivo-cerebellar involvement and GCIs. As shown in Table 5, pathogenicity is implicated with this LRRK2 variant.

TABLE 5
Clinical information for PD patients with LRRK2, I1371V variant

| | | | | | | Response to | | |
|---|---|---|---|---|---|---|---|---|
| Patient | Family | | Age of | Initial | Cardinal | dopaminergic | Additional | |
| ID | history | Gender | onset | symptoms | symptoms | therapy | features | Family history |
| 4862 | familial | unknown | 41 | micrographia, | AS, B, T | good response to | hypophonia, vivid | affected parent, AO 72 |
| (II-3) | (2 | | | right hand | | DA agonist | dreams | yrs, East Indian descent |
| | affected) | | | resting tremor | | (pramipexole) | | |
| MI- | familial | unknown | 33 | asymmetric | not reported | good response to | not reported | Mother affected |
| 007-01 | (2 | | | onset | | dopaminergic | | (deceased), carried |
| | affected) | | | | | therapy | | LRRK2, p.I1371V, |
| | | | | | | | | father affected (no |
| | | | | | | | | LRRK2 mutation) |
| MI- | | F | 61 | unilateral | AS, T (B, R, | good response to | severe cognitive | |
| 007-03 | | | (died | tremor and | PI not | dopaminergic | disturbances, visual | |
| | | | at age | motor | described) | therapy | hallucinations, | |
| | | | 71) | impairment | | | confirmed Lewy | |
| | | | | | | | body Parkinson's | |
| | | | | | | | disease | |
| Family | familial | F | 71, | bradykinesia, | B, R, T | moderate | MMSE 24/30, no | |
| C, | (3 | | duration | rest tremor | | response | asymmetiy | |
| III.1 | affected) | | 10 yrs | | | (150 mg | | |
| | | | | | | daily dose) | | |
| Family | | F | 48, | bradykinesia | AS, B, T | not reported | not reported | |
| C, | | | duration | | | | | |
| IV.2 | | | 6 yrs | | | | | |
| Family | familial | M | 42, | bradykinesia, | AS, B, R, T | good response | MMSE 28/30, | |
| D, | (2 | | duration | rest tremor, | | (900 mg | orthostatic | |
| II.3 | affected) | | 12 yrs | micrographia | | daily | hypotension | |
| | | | | | | dose) | | |
| N/A | sporadic | F | 57 | dragging of | AS, T | not reported | typical presentation | none |
| | | | | left leg, | | | of disease | |
| | | | | orthostatic | | | | |
| | | | | tremor | | | | |

AS = asymmetly at onset,
B = bradykinesia,
PI = postural instability,
R = rigidity,
T = tremor
MMSE = Mini Mental State Examination,
MOCA = Montreal cognitive assessment

The LRRK2, I1371V variant was first reported in 2005 in a family (case 4862) from East India with 2 affected first degree relatives (parent-child) with typical clinical presentation and good response to dopaminergic therapy. A detailed segregation analysis of additional family members was not possible, but this allele was not detected in180 controls from North America. The LRRK2, I1371V variant was next reported in an Italian family (MI-007) and showed segregation in two first degree relatives with asymmetric onset of symptoms and good response to dopaminergic therapy. Autopsy results in one case (MI-007-03) showed typical Lewy body pathology. In that study, one control was found to carry the LRRK2, I1371V variant out of a total of 416 control cases. Two additional families with the LRRK2, I1371V variants (Family C and Family D) were described with a total of five affected individuals. The clinical presentation as extracted from the clinical description was compatible with typical PD, with the variant segregated in the two families. Lastly, one sporadic PD case from Serbia was described by Jankovic et al. 2015 and another case without any detailed clinical description. These descriptions of clinical/pathological phenotype point towards a high risk variant in familial PD.

While MSA presents with glial inclusions as a key pathological hallmark, in the case described here, frequent alpha-synuclein-positive neuronal inclusions (both cytoplasmic and nuclear) in the substantia nigra, pontine nuclei, inferior olivary nucleus, and locus ceruleus (FIG. 4 A-D) were detected. In addition, neuritic pathology was detected in the most severely affected areas, especially the putamen, pontine base, and inferior olivary nucleus (FIG. 4 A-D).

The same genetic variant, LRRK2, I1371V, has been previously reported in an autopsy case with a pattern consistent with alpha-synuclein positive Lewy body pathology affecting primarily the nigrostriatal system (Table 5: patient ID MI-007-03).

Another example for the same allelic variant but different underlying pathologies is presented in the Japanese Sagamihara family with an LRRK2, p.Ile2020Thr mutation. Autopsies from eight affected mutation carriers presented in one case with multiple system atrophy (MSA-P), one case with typical Lewy-body pathology, whereas six cases in this family presented with pure nigral degeneration but no Lewy bodies. Alpha-synuclein accumulation was found both in neuronal and glia cell populations throughout the brainstem, this could be indicative of a common underlying disease pathway.

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

1-87. (canceled)

88. A method comprising:
a. assaying a sample for an LRRK2 genetic variation, wherein said LRRK2 genetic variation causes a LRRK2 mutation at position I1371, wherein said sample is from a subject suspected of having multiple system atrophy; and
b. detecting a presence of said LRRK2 genetic variation in said sample.

89. The method of claim 88, wherein said LRRK2 mutation at position I1371 is an I1371V mutation.

90. The method of claim 88, wherein said sample comprises a nucleic acid.

91. The method of claim 90, wherein said assaying comprises purifying said nucleic acid from said sample.

92. The method of claim 90, wherein said assaying comprises amplifying a nucleotide sequence of said sample.

93. The method of claim 90, wherein said assaying comprises a microarray analysis of said nucleic acid.

94. The method of claim 90, wherein said assaying comprises sequencing said nucleic acid.

95. The method of claim 88, wherein said sample is collected from blood, saliva, urine, serum, tears, skin, tissue, or hair.

96. The method of claim 88, wherein said subject has a symptom of multiple system atrophy.

97. The method of claim 88, wherein said subject is asymptomatic.

98. The method of claim 88, further comprising detecting multiple system atrophy in said subject.

99. The method of claim 98, wherein said detecting multiple system atrophy is based on an assessment by a medical doctor, a psychologist, a neurologist, a psychiatrist, or other professional who screens subjects for multiple system atrophy.

100. The method of claim 88, further comprising identifying said subject as having an increased risk of developing multiple system atrophy.

101. The method of claim 88, further comprising administering a treatment to said subject.

102. The method of claim 101, wherein said treatment comprises a pharmaceutical composition comprising an LRRK2 inhibitor.

103. A method for treating or preventing multiple system atrophy in a subject in need thereof comprising:
administering a therapeutically effective amount of an LRRK2 inhibitor, wherein said subject has an LRRK2 genetic variation, wherein said LRRK2 genetic variation does not cause an LRRK2 mutation at position N551, 1723, R1398, R1441, R1514, P1542, R1628, M1646, S1647, M1869, G2019, G2385, or T2397.

104. The method of claim 103, wherein said LRRK2 genetic variation causes an LRRK2 mutation at position I1371.

105. The method of claim 104, wherein said LRRK2 mutation at position I1371 is an I1371V mutation.

106. The method of claim 105, wherein said subject is at risk of developing multiple system atrophy.

107. The method of claim 106, wherein said method ameliorates a symptom of multiple system atrophy in said subject.
